(19) United States
(12) Patent Application Publication
(10) Pub. No.: US 2011/0152352 A1
(43) Pub. Date: Jun. 23, 2011

(54) SMAD PROTEINS CONTROL DROSHA-MEDIATED MIRNA MATURATION

(75) Inventors: Akiko Hata, Winchester, MA (US); Brandi Davis, Brighton, MA (US); Giorgio Lagna, Winchester, MA (US)

(73) Assignee: Tufts University, Boston, MA (US)

(21) Appl. No.: 12/997,402

(22) PCT Filed: Jun. 10, 2009
(86) PCT No.: PCT/US09/03494

§ 371 Date: Feb. 24, 2011

Related U.S. Application Data
(60) Provisional application No. 61/060,460, filed on Jun. 10, 2008.

(51) Int. Cl. A61K 31/713; C07H 21/02; C12N 15/63; C12N 7/01; C12N 5/071; C40B 40/06; A61P 35/00

(52) U.S. Cl. 514/44 A; 536/23.1; 435/320.1; 435/235.1; 435/325; 506/16

(57) ABSTRACT

The invention, in some aspects, relates to compositions and methods useful for modulating expression of miRNAs that are regulated by the TGF-β/BMP signaling pathway. In some aspects, the invention relates, to oligonucleotides comprising a CAGRN-motif that modulate expression of miRNAs that are regulated by TGF-β/BMP signaling pathway. The invention, in some aspects, relates to composition and methods useful for inhibiting microRNA processing. In some aspects, the invention relates to composition and methods for treating TGF-Beta/BMP mediated disorders.

### RELATED APPLICATIONS

This application claims priority under 35 U.S.C. §119 from U.S. provisional application Ser. No. 61/060,460, filed Jun. 10, 2008, the contents of which is incorporated herein in its entirety.

### GOVERNMENT FUNDING

This invention was made with Government support from the National Institutes of Health under Grant Nos. HD042149, HL082854, and HL086572. The Government has certain rights in the invention.

### FIELD OF THE INVENTION

The invention, in some aspects, relates to compositions and methods useful for modulating a TGF-β/BMP signaling pathway. In some aspects, the invention relates to oligonucleotides that modulate expression of miRNAs that are regulated by TGF-β/BMP signaling pathway. In some aspects, the invention relates to composition and methods for treating TGF-Beta/BMP mediated disorders.

### BACKGROUND OF THE INVENTION

MicroRNAs (miRNAs) are small non-coding RNAs that participate in the spatiotemporal regulation of mRNA and protein synthesis. Aberrant miRNA expression leads to developmental abnormalities and diseases, such as cardiovascular disorders and cancer; however, the stimuli and processes regulating miRNA biogenesis are largely unknown. The transforming growth factor β (TGF-β)/bone morphogenetic proteins (BMPs) family of growth factors orchestrates fundamental biological processes in development and in the homeostasis of adult tissues, including the vasculature. The involvement of miRNAs in TGF β/BMP signaling and TGF β/BMP mediated disorders has remained minimally understood.

### SUMMARY OF THE INVENTION

The invention generally relates to compositions and methods useful for modulating the TGF-β/BMP signaling pathway. It was found that miRNAs are regulated by and play a role in a TGF-β/BMP signaling pathway. It was also discovered that SMAD associates with a component of the Drosha microprocessor complex, generating responses involved in the TGF β/BMP signaling pathways.

The invention, in some aspects, provides isolated oligonucleotides comprising a substantially double-stranded portion having the nucleotide sequence CAGRN, wherein the isolated oligonucleotide inhibits the binding of a receptor-specific SMAD (rSMAD) protein to an miRNA, and wherein the isolated oligonucleotide is not a primary miRNA of miR-21, miR-199a, miR-105, miR-509-1(5p), miR-421, or miR-600r.

The invention, in some aspects, provides isolated oligonucleotides comprising a substantially double-stranded portion having the nucleotide sequence CAGRN, wherein the isolated oligonucleotide inhibits the binding of a receptor-specific SMAD (rSMAD) protein to an miRNA, and wherein at least one nucleotide is a modified nucleotide.

The invention, in some aspects, provides isolated oligonucleotides comprising a substantially double-stranded portion having the nucleotide sequence CAGRN, wherein the isolated oligonucleotide inhibits the binding of a receptor-specific SMAD (rSMAD) protein to an miRNA, and wherein the isolated oligonucleotide is conjugated to a Nuclear Localization Signal (NLS).

In some embodiments, the substantially double-stranded portion is entirely double-stranded in the nucleotide sequence CAGRN. In other embodiments, the substantially double-stranded portion has one mismatch in the nucleotide sequence CAGRN.

In some embodiments, where the isolated oligonucleotide inhibits the binding of a receptor-specific SMAD (rSMAD) protein to an miRNA, the rSMAD protein is selected from SMAD1, SMAD2, SMAD3, SMAD5 and SMAD8.

In some embodiments, where the isolated oligonucleotide inhibits the binding of a receptor-specific SMAD (rSMAD) protein to an miRNA, the miRNA is a primary miRNA.

In some embodiments, the isolated oligonucleotides have a formula selected from:

(SEQ ID NO: 3)
5′- (X¹)₍ᵢ₊ⱼ₎ C A G A C (X²)ₖG U C U G
(X³)₍ᵢ₊ₘ₎ -3′,
(SEQ ID NO: 4)
5′- (X¹)₍ᵢ₊ⱼ₎ G U C U G (X²)ₖC A G A C
(X³)₍ᵢ₊ₘ₎ -3′
and
(SEQ ID NO: 5)
5′- (X¹)₍ᵢ₊ⱼ₎ C A G A C (X²)ₖG U C G
(X³)₍ᵢ₊ₘ₎ -3′,

wherein each of the X¹X², and X³ is indenpendently any nucleotide, wherein i represents at least one nucleotide, wherein k represents at least one nucleotide, and wherein j and m independently represent zero or more overhang nucleotides, and wherein (X²)ₖ forms a loop structure.

In some embodiments, i and/or k represents up to about 10, 20, 30, 40, 50, 60, 70, 80, 90, or 100 or more nucleotides.

In some embodiments, i represents about 1 to 45 nucleotides.

In some embodiments, i represents 5 to 26 nucleotides.

In some embodiments, k represents about up to about 10, 20, 30, 40, 50, 60, 70, 80, 90, or 100 or more nucleotides.

In some embodiments, k represents 26 to 35 nucleotides.

In some embodiments, j and m independently represent 0, 1, 2, 3, 4, 5, or 6 overhang nucleotides.

In some embodiments, (X³)₍ᵢ₊ₘ₎ contains up to 0, 1, 2, 3, 4, 5, 6, 7, 8, 9, or 10 or more mismatches with the reverse complement of (X¹)₍ᵢ₊ⱼ₎.

In some embodiments, the isolated oligonucleotide comprises a sequence as set forth in SEQ ID NO: 73, SEQ ID NO: 74, SEQ ID NO: 75, SEQ ID NO: 76, SEQ ID NO: 77, or SEQ ID NO: 80.

The invention, in some aspects, provides vectors comprising any one or more of the isolated oligonucleotides disclosed herein and an expression sequence. The invention, in other aspects, provides cells comprising the vector. The invention, in still other aspects, provides viruses comprising the vector, optionally wherein the virus is an adenovirus, a lentivirus/retrovirus, herpesvirus, or a adeno-associated virus.

In some embodiments, the isolated oligonucleotides have formula selected from:

(SEQ ID NO: 6)
5′- (X¹)₍ᵢ₊ⱼ₎ C A G A C (X³)₍ₖ₊ₘ₎ -3′
(SEQ ID NO: 7)
3′- (X²)₍ᵢ₊ₙ₎ G U C U G (X⁴)₍ₖ₊ₚ₎ -5′,
(SEQ ID NO: 6)
5′- (X¹)₍ᵢ₊ⱼ₎ C A G A C (X³)₍ₖ₊ₘ₎ -3′
(SEQ ID NO: 8)
3′- (X²)₍ᵢ₊ₙ₎ G - C U G (X⁴)₍ₖ₊ₚ₎ -5′,
and
(SEQ ID NO: 6)
5′- (X¹)_{(i+f)} C A G A C (X³)₍ₖ₊ₘ₎ -3′
(SEQ ID NO: 9)
3′- (X²)₍ᵢ₊ₙ₎ - U C U G (X⁴)₍ₖ₊ₚ₎ -5′,

wherein each of X¹, X², X³, and X⁴, is independently any nucleotide, wherein i and k independently represent at least one nucleotide, and wherein j, n, m and p independently represent zero or more overhang nucleotides.

In some embodiments, i and/or k independently represent 1 to 20 nucleotides.

In some embodiments, i represents 5 to 16 nucleotides.

In some embodiments, k represents 6 to 13 nucleotides.

In some embodiments, j, n, m and p independently represent from 0 to 6 overhang nucleotides.

In some embodiments, each strand of the isolated oligonucleotide independently has a length of from 20 to 30 nucleotides.

In some embodiments, each strand of the isolated oligonucleotide independently has a length of from 21 to 27 nucleotides.

In some embodiments, (X²)₍ᵢ₊ₙ₎ is reverse complementary to (X¹)₍ᵢ₊ⱼ₎ at about 1 to i nucleotide positions.

In some embodiments, (X¹)₍ᵢ₊ⱼ₎ contains up to 0, 1, 2, 3, 4, 5, 6, 7, 8, 9, or 10 mismatches with the reverse complement of (X²)₍ᵢ₊ₙ₎.

In some embodiments, (X⁴)₍ₖ₊ₚ₎ is reverse complementary to (X³)₍ₖ₊ₘ₎ at about 1 to nucleotide positions.

In some embodiments, (X³)₍ₖ₊ₘ₎ contains up to 0, 1, 2, 3, 4, 5, 6, 7, 8, 9, or 10 mismatches with the reverse complement of (X⁴)₍ₖ₊ₚ₎.

In some embodiments, the isolated oligonucleotide comprises a sequence as set forth in SEQ ID NO: 73, SEQ ID NO: 74, SEQ ID NO: 75, SEQ ID NO: 76, SEQ ID NO: 77, or SEQ ID NO: 80.

In some embodiments, any of the isolated oligonucleotides disclosed herein have at least one nucleotide that is a modified nucleotide.

In some embodiments, any of the isolated oligonucleotides disclosed herein have at least one nucleotide that is an inosine or ribothymidine.

In some embodiments, any of the isolated oligonucleotides disclosed herein have at least one internucleotide bond that is a stabilized linkage, optionally wherein the stabilized linkage is a phosphonoacetate, a phosphorothioate, a phosphorodithioate, a methylphosphonate, a methylphosphorothioate, a 2′-5′ linkage, a peptide linkage, and dephospho bridge.

In some embodiments, any of the isolated oligonucleotides disclosed herein is conjugated to a Nuclear Localization Signal (NLS). In certain embodiments, the NLS is a peptide having a sequence as set forth in SEQ ID NO: 10, SEQ ID NO:11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO:19.

In some embodiments, any of the isolated oligonucleotides disclosed herein is conjugated to a lipid moiety. In certain embodiments, the lipid moiety is a cholesterol, cholesterol ester, fatty acid or glyceride.

The invention, in some aspects, provides compositions comprising any of the isolated oligonucleotides disclosed herein. In certain embodiments, the composition further comprises a pharmaceutically acceptable carrier.

The invention, in some aspects, provides methods for inhibiting maturation of at least one primary miRNA in a cell. The methods involve contacting the cell with any of the isolated oligonucleotides disclosed herein having a substantially double-stranded portion comprising the nucleotide sequence CAGRN, wherein the isolated oligonucleotide inhibits the binding of a receptor-specific SMAD (rSMAD) protein to a miRNA.

In some embodiments, the substantially double-stranded portion is entirely double-stranded in the nucleotide sequence CAGRN.

In some embodiments, the substantially double-stranded portion has one mismatch in the nucleotide sequence CAGRN.

In some embodiments, the rSMAD protein is selected from SMAD1, SMAD2, SMAD3, SMAD5 and SMAD8.

In some embodiments, the miRNA is a primary miRNA, optionally wherein the primary miRNA is a primary miRNA of miR-21, miR-199a, miR-105, miR-509-1(5p), miR-421, or miR-600.

In some embodiments, the inhibiting induces differentiation in the cell.

In some embodiments, the inhibiting inhibits differentiation in the cell.

In some embodiments, the inhibiting induces proliferation in the cell.

In some embodiments, the inhibiting inhibits proliferation in the cell.

In some embodiments, the cell is a stem cell, a cancer cell, a cancer stem cell, a smooth muscle precursor cell, a stromal cell or a fibroblastic cell. In certain embodiments, the cell is hematopoietic cell, a mesenchymal cell, or a neuronal cell.

In some embodiments, the contacting comprises transfecting the cell with a vector comprising any one or more of the isolated oligonucleotides disclosed herein and an expression sequence (i.e., an expression vector). In some embodiments, the transfecting comprises infecting the cell with a virus comprising the vector, optionally wherein the virus is an adenovirus, a lentivirus/retrovirus, herpesvirus, or a adeno-associated virus.

In some embodiments, the cell is in vitro.

In some embodiments, the cell is in vivo. In certain embodiments, the in vivo cell is in a human, a non-human primate, a mouse, a rat, a rabbit, a dog, a cat, a sheep, or a pig.

The invention, in some aspects, provides methods for treating a subject having a TGF-Beta/BMP mediated disorder. The methods involve administering to the subject a therapeutically effective amount of an isolated oligonucleotide comprising the nucleotide sequence CAGRN.

In some embodiments, the isolated oligonucleotide has a substantially double-stranded portion comprising the nucleotide sequence CAGRN. In certain embodiments, the substantially double-stranded portion is entirely double-stranded in the nucleotide sequence CAGRN. In certain other embodiments, the substantially double-stranded portion has one mismatch in the nucleotide sequence CAGRN.

In some embodiments, the isolated oligonucleotide inhibits the binding of a receptor-specific SMAD (rSMAD) protein to a miRNA. In certain embodiments, the rSMAD protein is selected from SMAD1, SMAD2, SMAD3, SMAD5 and SMAD8. In certain embodiments, the miRNA is a primary miRNA, optionally wherein the primary miRNA is a primary miRNA of miR-21, miR-199a, miR-105, miR-509-1(5p), miR-421, or miR-600.

In some embodiments, the TGF-β/BMP mediated disorder is a fibroproliferative disorder, a cancer, a smooth muscle cell disorder, an autoimmune disease, osteoarthritis, cardiovascular disorder, or scar tissue formation.

In certain embodiments, the smooth muscle related disorder is arterial hypertension or hereditary haemorrhagic telangiectasia.

In certain embodiments, the fibroproliferative disorder is selected from the group consisting of glomerulonephritis; diabetic nephropathy; renal interstitial fibrosis; pulmonary fibrosis; adult respiratory distress syndrome (ARDS); chronic obstructive pulmonary disease (COPD); idiopathic pulmonary fibrosis (TPF); acute lung injury (ALI); congestive heart failure; dilated cardiomyopathy; myocarditis; vascular stenosis; progressive systemic sclerosis; polymyositis; scleroderma; dermatomyositis; fascists; Raynaud's syndrome, rheumatoid arthritis; proliferative vitreoretinopathy; fibrosis associated with ocular surgery, and fibrotic skin conditions such as scleroderma and hypertrophic scar keloids.

In certain embodiments, the cancer is selected from the group consisting of: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; colorectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. In specific embodiments, the cancer is metastatic.

In some embodiments, the methods involve administering to the subject a therapeutically effective amount of any of the foregoing isolated oligonucleotides comprising the nucleotide sequence CAGRN.

In some embodiments, the methods involve administering to the subject a therapeutically effective amount of an isolated oligonucleotide having a formula of

(SEQ ID NO: 2)
5′- (X¹)ᵢ C A G A C (X²)ⱼ -3′
or
(SEQ ID NO: 2)
5′- (X¹)ᵢ G U C U G (X²)ⱼ -3′,

wherein each of X¹ and X² is independently any nucleotide, wherein i and j independently represent at least one nucleotide, and wherein the isolated oligonucleotide has a length of from 20 to 30 nucleotides.

In some embodiments, i and j independently represent from 1 to 20 nucleotides.

In some embodiments, i and j independently represent from 5 to 16 nucleotides.

In some embodiments, the isolated oligonucleotide has a length of from 21 to 27 nucleotides.

In some embodiments, the isolated oligonucleotide is a double stranded oligonucleotide with a first strand having a sequence of SEQ ID NO: 1 and a second strand having a sequence of SEQ ID NO: 2, which is at least partially complementary to SEQ ID NO: 1. In some embodiments, the first and second strands are not covalently linked. In some embodiments, the first and second strands are covalently linked.

In some embodiments, at least one nucleotide of the isolated oligonucleotide is a modified nucleotide.

In some embodiments, at least one nucleotide the isolated oligonucleotide is an inosine or ribothymidine.

In some embodiments, at least one internucleotide bond the isolated oligonucleotide is a stabilized linkage, optionally wherein the stabilized linkage is a phosphonoacetate, a phosphorothioate, a phosphorodithioate, a methylphosphonate, a methylphosphorothioate, a 2′-5′ linkage, a peptide linkage, and dephospho bridge.

In some embodiments, the isolated nucleotide is administered in a composition comprising a pharmaceutically acceptable carrier.

In some embodiments, the administering is intratumorally, intracranially, intravenously, intrapleurally, intranasally, intramuscularly, subcutaneously, intraperitoneally, or as an aerosol.

The invention, in some aspects, provides methods for treating cancer. The methods involve administering to the subject a therapeutically effective amount of an isolated oligonucleotide comprising the nucleotide sequence CAGRN.

In some embodiments, the isolated oligonucleotide has a substantially double-stranded portion comprising the nucleotide sequence CAGRN. In certain embodiments, the substantially double-stranded portion is entirely double-stranded in the nucleotide sequence CAGRN. In certain embodiments, the substantially double-stranded portion has one mismatch in the nucleotide sequence CAGRN.

In some embodiments, the isolated oligonucleotide inhibits the binding of a receptor-specific SMAD (rSMAD) protein to a miRNA. In certain embodiments, the rSMAD protein is selected from SMAD1, SMAD2, SMAD3, SMAD5 and SMAD8. In certain embodiments, the miRNA is a primary miRNA, optionally wherein the primary miRNA is a primary miRNA of miR-21, miR-199a, miR-105, miR-509-1(5p), miR-421, or miR-600.

In some embodiments, the methods involve administering to the subject a therapeutically effective amount of any of the foregoing isolated oligonucleotides comprising the nucleotide sequence CAGRN.

In some embodiments, the methods involve administering to the subject a therapeutically effective amount of an isolated oligonucleotide having a formula of

(SEQ ID NO: 1)
5′- (X¹)ᵢ C A G A C (X²)ⱼ -3′
or
(SEQ ID NO: 2)
5′- (X¹)ᵢ G U C U G (X²)ⱼ -3′,

wherein each of X¹ and X² is independently any nucleotide, wherein i and j independently represent at least one nucleotide, and wherein the isolated oligonucleotide has a length of from 20 to 30 nucleotides.

In some embodiments, i and j independently represent from 1 to 20 nucleotides.

In some embodiments, i and j independently represent from 5 to 16 nucleotides.

In some embodiments, the isolated oligonucleotide has a length of from 21 to 27 nucleotides.

In some embodiments, at least one nucleotide of the isolated oligonucleotide is a modified nucleotide.

In some embodiments, at least one nucleotide the isolated oligonucleotide is an inosine or ribothymidine.

In some embodiments, at least one internucleotide bond the isolated oligonucleotide is a stabilized linkage, optionally wherein the stabilized linkage is a phosphonoacetate, a phosphorothioate, a phosphorodithioate, a methylphosphonate, a methylphosphorothioate, a 2′-5′ linkage, a peptide linkage, and dephospho bridge.

In some aspects, the invention provides methods for treating cancer that involve administering to the subject a therapeutically effective amount of a SMAD inhibitor.

In some embodiments, the SMAD inhibitor is an MH1 or MH2 fragment.

In some embodiments, the SMAD inhibitor is an anti-SMAD antibody.

In some embodiments of the methods disclosed herein, the cancer is selected from the group consisting of: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; colorectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. In certain embodiments, the cancer is metastatic.

In some embodiments, the administering is intratumorally, intracranially, intravenously, intrapleurally, intranasally, intramuscularly, subcutaneously, intraperitoneally, or as an aerosol.

In some aspects, the invention provides methods for treating a smooth muscle cell disorder. The methods involve administering to the subject a therapeutically effective amount of an isolated TGF-β/BMP/miR pathway activator in an effective amount to treat the smooth muscle cell disorder in the subject.

In some embodiments, the isolated TGF-β/BMP/miR pathway activator is an exogenous TGF microRNA. In certain embodiments, the exogenous TGF microRNA is a vector encoding the microRNA.

In some embodiments, the isolated TGF-β/BMP/miR pathway activator is a SMAD. In certain embodiments, the SMAD is a rSMAD selected from SMAD1, SMAD2, SMAD3, SMAD5 and SMAD8. In certain other embodiments, the SMAD is a vector expressing SMAD.

In some embodiments, the smooth muscle cell disorder is selected from the group consisting of arterial hypertension, hereditary haemorrhagic telangiectasia, restenosis, atherosclerosis, coronary heart disease, thrombosis, myocardial infarction, stroke, smooth muscle neoplasms such as leiomyoma and leiomyosarcoma of the bowel and uterus.

The invention, in some aspects, provides methods for promoting wound healing in a subject. The methods involve administering to the subject a therapeutically effective amount of an isolated TGF-β/BMP/miR pathway activator in an effective amount to promote wound healing in the subject.

In some embodiments, the isolated TGF-β/BMP/miR pathway activator is an exogenous TGF microRNA. In certain embodiments, the exogenous TGF microRNA is a vector encoding the microRNA.

In some embodiments, the isolated TGF-β/BMP/miR pathway activator is a SMAD. In certain embodiments, the SMAD is a rSMAD selected from SMAD1, SMAD2, SMAD3, SMAD5 and SMAD8. In certain other embodiments, the SMAD is a vector expressing SMAD.

The invention, in some aspects, provides methods for treating a metabolic bone disorder in a subject. The methods involve administering to the subject a therapeutically effective amount of an isolated TGF-β/BMP/miR pathway activator in an effective amount to treat the metabolic bone disorder in the subject.

In some embodiments, the isolated TGF-β/BMP/miR pathway activator is an exogenous TGF microRNA. In certain embodiments, the exogenous TGF microRNA is a vector encoding the microRNA.

In some embodiments, the isolated TGF-β/BMP/miR pathway activator is a SMAD. In certain embodiments, the SMAD is a rSMAD selected from SMAD1, SMAD2, SMAD3, SMAD5 and SMAD8.

In some embodiments, the metabolic bone disorder is osteoporosis.

In some embodiments, the metabolic bone disorder is selected from the group consisting of osteopenia, Paget's Disease (osteitis deformans), osteomalacia, rickets, tumor-associated bone loss, hypophosphatasia, drug-induced osteomalacia, and renal osteodystrophy.

The invention, in some aspects, provides methods for treating a fibroproliferative disorder in a subject. The methods involve administering to the subject a therapeutically effective amount of an isolated TGF-β/BMP/miR pathway inhibitor in an effective amount to treat the fibroproliferative disorder in the subject.

In some embodiments, the fibroproliferative disorder is selected from the group consisting of glomerulonephritis; diabetic nephropathy; renal interstitial fibrosis; pulmonary fibrosis; adult respiratory distress syndrome (ARDS); chronic obstructive pulmonary disease (COPD); idiopathic pulmonary fibrosis (TPF); acute lung injury (ALI); congestive heart failure; dilated cardiomyopathy; myocarditis; vascular stenosis; progressive systemic sclerosis; polymyositis; scleroderma; dermatomyositis; fascists; Raynaud's syndrome, rheumatoid arthritis; proliferative vitreoretinopathy; fibrosis associated with ocular surgery, and fibrotic skin conditions such as scleroderma and hypertrophic scar keloids.

In some embodiments, the isolated TGF-β/BMP/miR pathway inhibitor is a TGF microRNA specific antisense.

In some embodiments, the isolated TGF-β/BMP/miR pathway inhibitor is a TGF microRNA sponge.

In some embodiments, the isolated TGF-β/BMP/miR pathway inhibitor is any of the isolated oligonucleotides disclosed herein.

In some embodiments, the isolated TGF-β/BMP/miR pathway inhibitor is a SMAD inhibitor. In certain embodiments, the SMAD inhibitor is a SMAD p-68 inhibitor.

The invention, in some aspects, provides methods for inhibiting scar tissue formation. The methods involve contacting a tissue with an isolated TGF-β/BMP/miR pathway inhibitor in an effective amount to inhibit scar tissue formation at the tissue.

In some embodiments, the isolated TGF-β/BMP/miR pathway inhibitor is a TGF microRNA specific antisense.

In some embodiments, the isolated TGF-β/BMP/miR pathway inhibitor is a TGF microRNA sponge.

In some embodiments, the isolated TGF-β/BMP/miR pathway inhibitor is a SMAD inhibitor. In certain embodiments, the SMAD inhibitor is a SMAD p-68 inhibitor.

The invention, in some aspects, provides methods for making a tissue engineering scaffold for inducing formation of extracellular matrix by cells bound to the scaffold comprising coupling an isolated TGF-β/BMP/miR pathway activator to the scaffold in an effective density to elicit production of extracellular matrix from the cells.

The invention, in some aspects, provides methods of inhibiting microRNA processing. In some embodiments, the methods involve contacting a cell with a SMAD inhibitor in an effective amount to inhibit processing of a TGF microRNA.

The invention, in some aspects, provides methods for inhibiting TGF-β signaling. The methods involve contacting a cell with a TGF-β/BMP/miR pathway inhibitor.

In some embodiments, the TGF-β/BMP/miR pathway inhibitor is an antisense oligonucleotide.

In some embodiments, the TGF-β/BMP/miR pathway inhibitor is a SMAD inhibitor.

In some embodiments, the TGF-β/BMP/miR pathway inhibitor is any of the foregoing isolated oligonucleotides.

According to further aspects of the invention, an miRNA is provided that comprises a heterologous substantially double-stranded portion comprising the nucleotide sequence CAGRN that promotes binding of a receptor-specific SMAD (rSMAD) protein to the miRNA, wherein R is A or G and N is A, G, C, or U.

In some embodiments, the miRNA further comprises a seed sequence that targets a gene associated with a TGF-β/BMP mediated disorder. In certain embodiments, the TGF-β/BMP mediated disorder is a fibroproliferative disorder, a cancer, or an autoimmune disease.

In some embodiments, the miRNA does not have a homologous substantially double-stranded portion having the nucleotide sequence CAGRN.

In some embodiments, the miRNA comprises at least one nucleotide that is a modified nucleotide or a deoxyribonucleotide.

In some embodiments, the miRNA is conjugated to a Nuclear Localization Signal (NLS).

According to some aspects, compositions are provided that comprise the miRNAs disclosed herein. In some embodiments, the compositions further comprise a pharmaceutically acceptable carrier.

According to other aspects of the invention, a synthetic miRNA is provided that comprises a seed sequence and a substantially double-stranded portion comprising the nucleotide sequence CAGRN that promotes binding of a receptor-specific SMAD (rSMAD) protein to the synthetic miRNA, wherein R is A or G and N is A, G, C, or U.

In some embodiments, the seed sequence targets a gene associated with a TGF-β/BMP mediated disorder. In certain embodiments, the TGF-β/BMP mediated disorder is a fibroproliferative disorder, a cancer, or an autoimmune disease.

In some embodiments, the synthetic miRNA comprises at least one nucleotide that is a modified nucleotide or a deoxyribonucleotide.

In some embodiments, the synthetic miRNA is conjugated to a Nuclear Localization Signal (NLS).

According to some aspects, compositions are provided that comprise the synthetic miRNAs disclosed herein. In some embodiments, the compositions further comprise a pharmaceutically acceptable carrier.

According to some aspects of the invention, methods are provided for detecting aberrant TGF/BMP signaling in a subject. In some embodiments, the methods comprise obtaining a biological sample of the subject, determining levels in the sample of a plurality of TGF miRNAs, and if levels of at least a subset of the TGF miRNAs are above control levels, detecting aberrant TGF/BMP signaling in the subject. In some embodiments, the plurality of TGF miRNAs is at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, or more TGF miRNAs. In some embodiments, the TGF miRNAs are selected from Table 2. In some embodiments, the subset of the TGF miRNAs are at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, or more of the plurality. In some embodiments, the subset of the TGF miRNAs represent about 1%, about 5%, about 10%, about 20%, about 30%, about 40%, about 50%, about 60%, about 70%, about 80%, about 90%, or more of the plurality.

In some embodiments, detection of aberrant TGF/BMP signaling is predictive of the subject having a TGF-β/BMP mediated disorder. In certain embodiments, the TGF-β/BMP mediated disorder is a fibroproliferative disorder, a cancer, or an autoimmune disease. In one embodiment, the TGF-β/BMP mediated disorder is a cancer. In some embodiments, aberrant TGF/BMP signaling in the cancer is indicative of a metastatic cancer.

In some embodiments, the TGF miRNAs are selected from the group consisting of: hsa-miR-21, hsa-miR-148a, hsa-miR-18a, hsa-miR-127-5p, hsa-miR-23a, hsa-miR-105, hsa-miR-148b, hsa-miR-106b, hsa-miR-134, hsa-miR-23b, hsa-miR-199a-5p, hsa-miR-152, hsa-miR-410, hsa-miR-103, hsa-miR-195, hsa-miR-542-3p, hsa-miR-107, hsa-miR-215, hsa-miR-339-3p, hsa-miR-140-3p, hsa-miR-342-3p, hsa-miR-423-5p, hsa-miR-421, hsa-miR-361-5p, hsa-miR-452, hsa-miR-509-5p, hsa-miR-331-5p, hsa-miR-345, hsa-miR-600, hsa-miR-422a, hsa-miR-518e, hsa-miR-487a, hsa-miR-631, hsa-miR-487b, and hsa-miR-654-5p.

In some embodiments, the control level of a TGF miRNA is the level of the TGF miRNA in a tissue that does not have aberrant TGF/BMP signaling (e.g., a healthy tissue, a non-metastatic cancer). In some embodiments, levels that are at least 1.5 fold, at least 2 fold, at least 3 fold, at least 4 fold, at least 5 fold or more above control levels are indicative of aberrant TGF/BMP signaling.

According to some aspects of the invention, oligonucleotide arrays for determining levels of miRNAs are provided. In some embodiments, the oligonucleotide arrays consist essentially of immobilized probes that hybridize with TGF miRNAs, and optionally one or more control probes. In some embodiments, TGF miRNAs that hybridize with immobilized probes are selected from the group consisting of: hsa-miR-21, hsa-miR-148a, hsa-miR-18a, hsa-miR-127-5p, hsa-miR-23a, hsa-miR-630, hsa-miR-105, hsa-miR-148b, hsa-miR-106b, hsa-miR-134, hsa-miR-23b, hsa-miR-648, hsa-miR-199a-5p, hsa-miR-152, hsa-miR-410, hsa-miR-198, hsa-miR-103, hsa-miR-659, hsa-miR-214, hsa-miR-195, hsa-miR-542-3p, hsa-miR-330-3p, hsa-miR-107, hsa-miR-671-3p, hsa-miR-215, hsa-miR-298, hsa-miR-607, hsa-miR-339-3p, hsa-miR-140-3p, hsa-miR-770-5p, hsa-miR-300, hsa-miR-342-3p, hsa-miR-1298, hsa-miR-423-5p, hsa-miR-188-3p, hsa-miR-877, hsa-miR-421, hsa-miR-361-5p, hsa-miR-1539, hsa-miR-452, hsa-miR-220c, hsa-miR-933, hsa-miR-509-5p, hsa-miR-378, hsa-miR-508-5p, hsa-miR-331-5p, hsa-miR-940, hsa-miR-509-3-5p, hsa-miR-383, hsa-miR-516a-3p, hsa-miR-345, hsa-miR-1205, hsa-miR-600, hsa-miR-422a, hsa-miR-518e, hsa-miR-487a, hsa-miR-1207-5p, hsa-miR-631, hsa-miR-541, hsa-miR-520a-5p, hsa-miR-487b, hsa-miR-1266, hsa-miR-1208, hsa-miR-567, hsa-miR-525-5p, hsa-miR-498, hsa-miR-1290, hsa-miR-1284, hsa-miR-654-5p, hsa-miR-922, hsa-miR-513a-5p, hsa-miR-1321, hsa-miR-1292, hsa-miR-921, hsa-miR-1912, hsa-miR-612, hsa-miR-1909, hsa-miR-1324, hsa-miR-1324, hsa-miR-623, and hsa-miR-1915. In some embodiments, the oligonucleotide arrays consist of probes that hybridize with up to 10, up to 20, up to 30, up to 40, up to 50, up to 100, up to 200, up to 300, up to 400, up to 500, up to 1000 or more different miRNAs.

According to still other aspects of the invention, methods of inducing miRNA activity are provided. In some embodiments, the methods comprise contacting a cell with an miRNA disclosed herein, e.g., a synthetic miRNA, and contacting the cell with a BMP or rSMAD to induce miRNA activity. In some embodiments, the cell is contacted with an expression vector for expressing the miRNA. In some embodiments, the cell is in vitro. In other embodiments, the cell is in vivo.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 depicts that miR-21 is critical for the modulation of VSMC phenotype by BMP. a. Level of expression of miRNAs normalized to U6 snRNA in PASMCs treated with BMP4 for 24 h (*P<0.05; n=4). b. PASMCs transfected with antisense RNA oligonucleotides against different miRNAs or GFP (control). After BMP4 treatment (48 h), cells were stained with anti-SMA antibody (left images) and DAPI (right images). c. PASMCs infected with adenovirus carrying CMV-driven GFP (control; Ad-GFP), miR-21 (Ad-miR-21) or miR-125b (Ad-miR-125b). SMA mRNA level was measured after BMP4 treatment (48 h) (*P<0.05; n=4). d. 10T1/2 cells transfected with vector (mock) or human PDCD4 cDNA construct, followed by BMP4 treatment (24 h). Expression of SMA, calponin, SM22a, Id3, or hPDCD4 relative to GAPDH mRNA is shown. (*P<0.001, n=3) e. PASMCs transfected with control siRNA (siCtr) or siRNA for PDCD4 (siPDCD4). Relative mRNA expression shown as in (d).

FIG. 2 depicts post-transcriptional regulation of miR-21 biosynthesis by the TGFβ pathway. a. Expression of mature miR-21 and miR-199a normalized to U6 snRNA in PASMCs stimulated with BMP4 or TGFβ1 (24 h). (*P<0.05; n=3). b. Time-course of pri-, pre- or mature miR-21 (pri/pre/mat-miR-21) expression in PASMCs upon stimulation with BMP4 or TGFβ1. c. PASMCs pretreated with α-amanitin were stimulated with BMP4 (5 h). Expression of pri/pre/mat-miR-21 or Id1 is shown (*P<0.05; n=3). d. Relative expression of pri/pre/mat-miR-21 derived from increasing amounts of human miR-21 expression construct (pCMV-miR-21) transfected into 10T1/2 cells. (*P<0.05; n=3).

FIG. 3 depicts interaction of Smads with p68, a component of the Drosha complex. a. PASMCs were transfected with control siRNA (siCtr) or a mixture of siRNAs for Smad1 and Smad5 (siSmad). After BMP4 treatment (2 h), the expression of pri/pre/mat-miR-21 was compared (top panel). As controls, expression of Id3, Smad1, Smad5, and SMA is shown (bottom panel). b. PASMCs were transfected with control siRNA (siCtr) or siRNAs for p68 (si-p68). Expression of pri/pre/mat-miR-21 was examined after BMP4 treatment (2 h). (*P<0.05; n=3). c. Nuclear extracts prepared from PASMCs treated with BMP4 (2 h) and subjected to immunoprecipitation with anti-p68, anti-Drosha antibody, or non-specific IgG (control), followed by immunostaining with anti-Smad1/5, anti-p68, or anti-Drosha antibody. Nuclear extracts were immunostained with anti-lamin A/C antibody (control).

FIG. 4 depicts association of Smads with pri-miRNA promotes processing by Drosha. a. Cos7 cells transfected with pCMV-miR-21 and Flag-Smad1, Flag-Smad3, or Flag-Smad2, followed by BMP4 or TGFβ1 treatment (2 h). RNA-ChIP performed with anti-Flag antibody or non-specific IgG (control), followed by PCR amplification with miR-21 primers. (*P<0.05, compared to no treatment; n=4). b. After treatment of PASMCs with BMP4 or TGFβ1 (1 h), endogenous Smad1/Smad5, Smad2/Smad3, p68 or Drosha were immunoprecipitated and subjected to PCR analysis with miR-21, miR-199a, or miR-214 primers. As controls, RNA samples untreated with RT (-RT) or immunoprecipitated with non-specific IgG (IgG) were subjected to PCR. (*P<0.05 compared to none; n=4). c. In vitro pri-miRNA processing assay performed by incubating pri-miR-21 substrate with the nuclear extracts prepared from Cos7 cells treated with vehicle, BMP4 or TGFβ1 (2 h).

FIG. 5 depicts smad4-independent mechanism of maturation of pri-miRNA. a. Level of expression of pri/pre/mat-miR-21 or Smad4 after treatment with BMP4 (2 h) in PASMCs transfected with control siRNA (siCtr) or Smad4 siRNA (siS4). b. Level of expression of pri/pre/mat-miR-21 or PAI-1 in Smad4-negative breast carcinoma MDA-MB-468 cells stimulated with TGFβ1 (0.5 h). (*P<0.05; n=3). c. MDA-MB-468 cells were treated with TGFβ1 (1 h) prior to RNA-ChIP. Endogenous proteins were precipitated with anti-Smad1/Smad5, anti-Smad2/Smad3 or anti-Drosha antibodies, followed by PCR analysis with a miR-21 primer. (*P<0.05, compared to none; n=3). d. MDA-MB-468 cells were infected with adenovirus carrying dominant-negative type I TGFβ receptor (dnALK5), an inhibitor of TGFβ signaling, prior to TGFβ treatment (1 h). The amount of pri/pre/mat-miR-21 was examined. (*P<0.05, compared to none; n=3).

FIG. 6 depicts miRNAs cloned in PASMCs treated with BMP4. Percentage of miRNAs cloned under mock or BMP4 treated hPASMCs are indicated. ‘others’ includes miR-24, 25, 136, 379, 146a, 23b, 26a, 27a, 30a-5p, 493-3p, let7a, let7b, let7c, let7f, and let71).

FIG. 7 depicts miR-21 expression is similarly induced upon stimulation with various BMP ligands. PASMCs were treated with or without 0.3, 1, or 3 nM BMP4, BMP2, or BMP7 for 24 hr and subjected to qRT-PCR analysis using miR-21 primers. The induction of mature miR-21 is shown as a ratio to samples not treated with BMPs. Average of three experiments each performed in triplicate with standard errors are presented.

FIG. 8 depicts anti-miR-21 specifically downregulates miR-21 expression. hPASMCs were transfected with antisense oligonucleotides (106 nM) of miR-21 or GFP (control). The expression level of endogenous miR-21 and miR-125a relative to U6 snRNA were examined by qRT-PCR. Average of three experiments each performed in triplicate with standard errors are presented.

FIG. 9 depicts inhibition of miR-21 upregulates SM-specific gene markers in both SMCs and non-SMCs. a. hPASMCs transfected with antisense oligonucleotides (106 nM) against GFP (control), miR-21 or miR-125b were subjected to immunofluorescence staining by FITC-conjugated anti-SMA or anti-calponin. Nuclei are visualized by DAPI staining. b. Total RNAs prepared from hPASMCs transfected with antisense oligonucleotides (anti-miR) (106 nM) against GFP (control), miR-21, miR-125a, miR-125b, miR-221, miR-15b, or miR-100 were subjected to qRT-PCR analysis of SMA, calponin, and Id3 gene. Values labeled with the same letters do not differ significantly from one another (P<0.05). Average of three experiments each performed in triplicate with standard errors are presented. c. Mouse 10T1/2 cells were transfected with anti-miR-21, anti-miR-125a or anti-GFP (control). Cells were then treated with BMP4 (3 nM) for 48 hr and subjected to immunofluorescence staining with FITC-conjugated anti-SMA monoclonal antibody (left images) and nuclear staining with DAPI (right images).

FIG. 10 depicts that miR-21 is critical for modulation of VSMC phenotype by the TGFβ signaling pathway. a. hPASMCs were infected with adenovirus carrying CMV driven GFP (control; Ad-GFP), miR-21 (Ad-miR-21) or miR-125b gene (Ad-miR-125b). Twenty-four hr after infection, cells were treated with BMP4 (3 nM) or vehicle for 48 hr, followed by immunofluorescence staining with FITC-conjugated anti-SMA monoclonal antibody (left images) and nuclear staining with DAPI (right images). Mature miR-21 and miR-125b expression normalized to U6 snRNA in PASMCs infected with adenovirus (Ad-GFP, AdmiR-21, or Ad-miR125b) were examined by qRT-PCR analysis. Average of three experiments each performed in triplicate with standard errors are presented.

FIG. 11 depicts that PDCD4 is a functional target of miR-21 in the regulation of SM phenotype by BMP. a. hPASMCs were infected with adenovirus carrying miR-21 (AdmiR-21) or two controls GFP (control; Ad-GFP) and miR-125b gene (Ad-miR-125b). Twenty-four hr after infection, cells were treated with BMP4 (3 nM) or vehicle for 48 hr. PDCD4 mRNA expression normalized to GAPDH was examined by qRT-PCR analysis. b. The level of expression of PDCD4 normalized to GAPDH in hPASMCs transfected with antisense oligonucleotides (106 nM) against miR-21 or two controls GFP and miR-221 was examined by qRT-PCR analysis. c. Schematic diagram of a human PDCD4 expression construct, which includes a miR-21 target sequence in its 3′-untranslated region (UTR).

FIG. 12 depicts a time-course expression assay of mature miR-21, miR-199a, or miR-221. The level of expression of miR-21, miR-199a, or miR-221 normalized to U6 snRNA was examined by qRT-PCR in PASMCs stimulated with 3 nM BMP4 or 400 μM TG931 for 0.25-24 hr as indicated.

FIG. 13 shows post-transcriptional regulation of miR-21 expression by the TGFβ pathway. a. hPASMCs were treated with or without 3 nM BMP4, BMP2 or 400 pM TGFβ1 for 24 hr and subjected to qRT-PCR analysis using mature miR-21, pri-miR-21 or pre-miR-21 primers. b. Schematic representation of the miR-21 sensor construct. Each red circle represents a sequence complementary to miR-21 cloned within the 3′-UTR of the luciferase gene. 10T1/2 cells were transfected with miR-21 sensor construct (1 μg), miR-21 expression construct (5 or 50 ng) and LacZ construct (100 ng). After treatment with BMP4 (3 nM) for 24 hr, cell were harvested and subjected to luciferase and LacZ assay. The luciferase activity normalized to LacZ activity is expressed in arbitrary units.

FIG. 14 depicts that transcriptional activity of miR-21 promoter is not affected by BMP4 or TGFβ stimulation. 10T1/2 cells were transfected with the miR-21 promoter-Luc construct (1 μg) and a LacZ reporter (100 ng). After treatment with BMP4 (3 nM) or TGFβ1 (400 pM) for 24 hr, cell were harvested and subjected to luciferase and LacZ assay. The luciferase activities, normalized to LacZ, were plotted in arbitrary units. Constitutive active Stat3 (ca-Stat3), which activates miR-21 transcription, was used as positive control. Average of three experiments each performed in triplicate with standard errors are presented.

FIG. 15 depicts that induction of SMA by CMV-miR-21 expression construct is BMP-dependent. a. Increasing amounts (50, 100, 250 or 500 ng) of miR-21 precursor construct (pCMV-miR-21) was transfected into 10T1/2 cells. The relative SMA expression level was measured by qRT-PCR normalized to GAPDH. Average of three experiments each performed in triplicate with standard errors are presented. Asterisks indicate statistically significant difference in expression (P<0.001). b. 10T1/2 cells transfected with increasing amounts of pCMV-miR-21 construct (50, 100, or 250 ng) were treated with BMP4 (3 nM) or vehicle for 48 hr. Total cell lysates were subjected to immunoblot with anti-SMA monoclonal antibody. The result is shown at two different exposure times (short exp. and long exp.). To control for loading variation, the same membrane was blotted with anti-GAPDH monoclonal antibody.

FIG. 16 depicts that downregulation of Smad1/Smad5 proteins by siRNA. hPASMCs were transfected with non-targeting control siRNA (siCtr) or mixture of siRNAs for Smad1 and Smad5 (siSmad). Twenty-four hr after transfection, cells were treated with 3 nM BMP4 for 2 hr, and subjected to immunoblotting analysis using anti-Smad1/5 antibodies or anti-GAPDH for loading control.

FIG. 17 depicts that downregulation of p68 does not affect induction of Id3 by BMP4. a. PASMCs were transfected with non-targeting control siRNA (siCtr) or siRNAs for p68 (si-p68). Twenty-four hr after transfection, cells were treated with 3 nM BMP4 for 2 hr. Total cell lysates were subjected to immunoblotting with anti-p68 antibody. Immunoblot with anti-GAPDH antibody is shown as loading control. b. RNA samples from PASMCs transfected with si-p68 or siCtr were subjected to qRT-PCR analysis to examine levels of expression of p68 or Id3 normalized to GAPDH.

FIG. 18 depicts ligand-dependent interaction of R-Smads with p68, a subunit of the Drosha microprocessor complex. a. Cos7 cells were transfected with Flag-tagged Smad1 and/or p68 construct. After BMP4 treatment for 2 hr, total cell lysates were prepared and subjected to immunoprecipitation with anti-Flag monoclonal antibody. Interaction of p68 was examined by immunostaining with anti-p68 monoclonal antibody. Immunoblot with anti-GAPDH antibody is shown as loading control. b. Cos7 cells were transfected with Flag-tagged Smad1 and/or Myc-tagged Drosha construct. After BMP4 treatment for 2 hr, total cell lysates were prepared and subjected to immunoprecipitation with anti-Myc monoclonal antibody. Interaction of Smad1 or p68 was examined by immunostaining with anti-Flag or anti-p68 monoclonal antibody.

FIG. 19 depicts partially purified GST-Smad fusion proteins. a. Nuclear extracts were prepared from Cos7 cells transfected with p68, and mixed with GST alone or GSTSmad1, GST-Smad3, GST-Smad4 or GST-Smad5 fusion proteins. Proteins interacting with GST fusion proteins were precipitated and subjected to immunoblot with anti-p68 antibody (top panel). Partially purified GST-Smad1, GST-Smad4, or GST-Smad5 fusion proteins are shown by Coomassie Blue staining of the gel (bottom panel). b. Nuclear extracts were prepared from Cos7 cells transfected with p68, and mixed with GST alone or GST-Smad 1 fully length (FL), the MH1 domain, the MH2 domain, GST-Smad3(FL), GST-Smad4(FL) or GST-Smad4(MH2) fusion proteins. Proteins interacting with GST fusion proteins were precipitated and subjected to immunoblot with anti-p68 antibody (top panel). Partially purified GST-Smad1 (FL, MH1 or MH2), GST-Smad3(FL), or GST-Smad4(FL or MH2) fusion proteins are shown by Coomassie Blue staining of the gel (bottom panel).

FIG. 20 depicts that interaction between R-Smads and p68 does not require an association with pri-miRNA. Nuclear extracts prepared from Cos7 cells transfected with p68 or Drosha were treated with RNase A (250 μg/ml) for 30 min prior to addition of GST alone or GST-Smad1, GST-Smad3, GST-Smad4 or GST-Smad5 fusion proteins. Proteins interacting with GST fusion proteins were precipitated and subjected to immunoblot with anti-p68 or anti-Drosha antibody.

FIG. 21 depicts ligand-dependent association of Smads with pri-miR-21. a. Schematic representation of pri-miR-21 with pre-miR-21 shown as the hairpin structure in red. Arrow indicates transcription initiation site for miR-21 gene. Two PCR primers (TM and miR-21) used in RNA-ChIP assays are indicated. As the miR-21 gene is located in the 3′-UTR of the transmembrane protein 49 gene (TMEM49), a primer set recognizing the TMEM49 coding region (TM) was used as negative control. Cos7 cells were transfected with pCMV-miR-21 and Flag-Smad1, Flag-Smad1 (3SA), or Flag-Smad3, followed by BMP4 treatment for 2 hr. RNA-ChIP analysis was performed by immunoprecipitation of RNA fragments with anti-Flag antibody or non-specific IgG (IgG), followed by PCR amplification with miR-21 primers. As negative control, RNA sample from Flag-Smad1 cells untreated with RT was subjected to PCR (-RT). * P<0.05 (compared to no treatment). b. RNA fragments were pulled down with GST alone or GST-full-length Smad1, Smad5, Smad3, Smad4 or the MH1 or MH2 domain of Smad1 fusion proteins. After precipitation, association of pri-miR-21 with these proteins was accessed by PCR analysis using miR-21 primers. *P<0.001 (compared to GST).

FIG. 22 depicts expression of different Pri-miRNAs and Pre-miRNAs of input RNA used in RNA-ChIP analysis. PASMCs were treated with BMP4 or TGFβ for 1 hr. Total RNA (input) was subjected to qRT-PCR analysis to examine Pri-miR-21 or PremiR-21 expression before and after BMP4 or TGFβ stimulation prior to RNA-ChIP analysis shown in FIG. 4b. * P<0.05 (compared to none).

FIG. 23 depicts that Smad4 is essential for transcriptional activation of Id3 gene. a. PASMCs were transfected with non-targeting control siRNA (siCtr) or siRNA for Smad4 (siS4). Twenty-four hr after transfection, cells were treated with 3 nM BMP4 for 2 hr. Total cell lysates were subjected to immunoblot with anti-Smad4 or anti-GAPDH (loading control) antibody. b. RNA samples were subjected to qRT-PCR analysis to measure the level of expression of Id3 normalized to GAPDH.

FIG. 24 depicts post-transcriptional induction of pri-miR-21 processing and accumulation of pre-miR-21 by BMP4 in breast carcinomas. A time-course expression of pri-miR-21, pre-miR-21, or mature miR-21 in human breast carcinoma MDA-MB-468 and MCF7 cells stimulated with 3 nM BMP4 for 0, 15, 30, 60, 90 or 120 min. Average of three experiments each performed in triplicate with standard errors are presented.

FIG. 25 depicts accumulation of Pre-miR-21 but not mature miR-21 by TGFβ in breast carcinoma MDA-MB-231 cells. Expression of pri-miR-21, pre-miR-21, mature miR-21 or PAI-1 in breast carcinoma MDA-MB-231 cells stimulated with 400 pM TGFβ1 for 1.5 hr was examined by qRT-PCR. Average of three experiments each performed in triplicate with standard errors are presented.

FIG. 26 depicts Smad4-independent association of R-Smads and Drosha with pri-miR-199a. MDA-MB-468 cells were treated with TGFβ1 (400 pM) for 1 hr prior to RNA-ChIP analysis. Endogenous Smad1/Smad5, Smad2/Smad3, or Drosha were precipitated with anti-Smad1/Smad5 anti-Smad2/Smad3 antibodies or anti-Drosha antibodies, followed by PCR analysis using primer sets for miR-199a, or miR-214 (control) (top panel). Non-specific IgG was used as negative control. * P<0.05 (compared to none). Total RNA (input) was subjected to qRT-PCR analysis to examine a level of expression of pri-miRNA or pre-miRNA of miR-21, miR-199a, or miR-214 expression before and after BMP4 or TGFβ1 stimulation (bottom panel).

FIG. 27. depicts the stem-loop (hairpin) structure of miRNAs containing CAGAC sequences in the region of their mature miRNA. Mature miRNA sequences are denoted by brackets and R-SBE sequences are shaded. These TGF miRNAs where identified through a combination of computational sequence searching of miRNA databases with visual inspection of sequences to identify miRNAs containing CAGAC sequences (i.e., TGF miRNAs) and, in some cases, tested for TGFβ/BMP responsiveness (See FIG. 28 and Example 9, for example). FIG. 27A shows TGF miRNAs Hsa-mir-21 (SEQ ID NO: 66), Hsa-miR-199a (SEQ ID NO: 63), Hsa-miR-105 (SEQ ID NO: 65), Hsa-miR-509-5p (SEQ ID NO: 66), Hsa-miR-421 (SEQ ID NO: 69), which are strongly regulated by TGFβ/BMP in PASMC and/or MDA468 cells. FIG. 27B shows Hsa-miR-215 (SEQ ID NO: 70) (weak slow response in MDA468) which is weakly regulated by TGFβ/BMP in PASMC and/or MDA468 cells. FIG. 27C shows Hsa-miR-214 (SEQ ID NO: 71) and Hsa-miR-600 (SEQ ID NO: 72) which are not regulated by TGFβ/BMP in PASMC and/or MDA468 cells. FIG. 27D shows Hsa-miR-631 (SEQ ID NO: 158), Hsa-miR-300 (SEQ ID NO: 159), Mmu-miR-686 (SEQ ID NO: 160), Mmu-miR-717 (SEQ ID NO: 161), Mmu-miR-743b (SEQ ID NO: 162), Mmu-miR-220 (SEQ ID NO: 163), and Mmu-miR-466g (SEQ ID NO: 164) which were identified but not tested for TGFβ/BMP regulation. FIG. 27E shows Hsa-miR-18a (SEQ ID NO: 165), Hsa-miR-106b (SEQ ID NO: 166), Hsa-miR-410 (SEQ ID NO: 167), Hsa-miR-542 (SEQ ID NO: 168), Hsa-miR-607 (SEQ ID NO: 169), and Hsa-miR-871 (SEQ ID NO: 170) microRNAs with CAGAT. These CAGAT microRNAs were identified but not tested for TGFβ/BMP regulation. Additional viral miRs with CAGAC were identified but not tested; these include mghv-miR-M1-2, ebv-miR-BART-11-5p, and rlcv-miR-rL1-12-5p (hairpins not shown).

FIG. 28. depicts the expression of TGF miRNAs following BMP-4 treatment in human pulmonary artery smooth muscle cells (PASMC).

FIG. 29. depicts the interaction of SMAD proteins with double stranded CAGAC sequences of miRNA by RNA pull down experiments. Full length GST-SMAD1 fusion protein and a GST-SMAD1 N-terminal (n219) MH1 domain interact with double stranded CAGAC sequences of miR-21 [gst: GST (tag) protein, s1: GST—Smad1 full length fusion protein, s4: GST—Smad4 full length fusion protein, s5: GST—Smad5 full length fusion protein, s1—c204: GST—Smad1 (MH2 domain) fusion protein, s1—n219: GST—Smad1 (MH1 domain) fusion protein]

FIG. 30 depicts an miRNA array analysis of TGFβ or BMP regulated miRNAs in PASMC. A. The fold induction of microRNAs following 24H TGFβ or BMP4 treatment compared to mock treated PASM was sorted by k-means clustering using Gene Pattern, and displayed by heatmap. Cluster 1 contains miRNAs induced by both TGFβ and BMP4. Cluster 2 contains miRNAs that are induced primarily by BMP4 treatment. Cluster 3 contains miRNA that are induced primarily by TGFβ. Cluster 4 contains miRNAs that are downregulated by BMP4 and/or TGFβ. B. Sequence logo representing the conserved motif present in miRNAs in Cluster 1 is indicated.

FIG. 31 depicts the identification of novel miRNAs regulated by the TGFβ signaling pathway post-transcriptionally. A. Sequence alignment of miRNAs containing R-SBE (boxed sequences) which were studied (left panel). Levels of expression of mature miRNAs were examined in hPASMCs with BMP4 or TGFβ1 treatment for 4 hr (right panel). miR-25 does not contain R-SBE and is not regulated by BMP4 or TGFβ1. Fold induction after treatment is presented, normalized to mock treated PASMC. B. hPASMCs (left panel) or human breast carcinoma MDA-MB-468 cells (right panel) were treated with 3 nM BMP4 or 400 pM TGFβ1 for 2 hr and subjected to qRT-PCR analysis primers specific for pre-miRNAs. Fold induction relative to mock treated sample is presented. Expression of all pre-miRNAs was elevated over 2-fold upon TGFβ1 or BMP4 treatment. Changes of pre-miR-25 upon TGF (31 or BMP4 treatment were not significant. C. A time-course expression of pri-miR-21, -105, -199a, -215, -421 or -509 was examined by qRT-PCR in PASMCs stimulated with 3 nM BMP4 (left panel) or 400 pM TGFβ1 (right panel) for 2 or 4 hr. Levels of expression of pri-miRNAs were normalized to GAPDH. Fold induction compared to untreated samples are presented.

FIG. 32 depicts an RNA-IP assay that was performed in hPASMCs treated for 2H with BMP4 followed by immunoprecipitaion of RNA fragments with anti-Smad1/5 antibody, anti-Drosha antibody anti-DGCR8 antibody, or non-specific IgG (IgG), and PCR amplification with indicatedprimers. Fold induction of binding relative to untreated PASMC is presented. Primers for miR-214 and -222, TM serve as negative controls as they are not regulated by TGFβ or BMP.

FIG. 33 shows that Smad is essential for recruitment of Drosha. hPASMCs were transfected with non-targeting control siRNA (si-Control) or mixutre of siRNAs for Smad1 and Smad5 (si-Smads). Twenty-four hr after transfection, cells were treated with 3 nM BMP4 for 2 hr, and subjected to RNA-IP analysis to examine recruitment of Drosha to different pri-miRNAs (pri-miR-21, -105, -199a, -421, or -221). Amounts of pri-miRNAs were examined after immunoprecipitation of RNA fragments with anti-Drosha antibody (top panel). The expression of pre-miRNAs in si-smad treated cells was quantitated by qRT-PCR analysis (bottom panel). As controls, level of Id3 mRNA was monitored (right panel).

FIG. 34 depicts that the SBE-like sequence found in the TGFβ/BMP-regulated miRNAs is essential for the regulation of the Drosha processing. A. Schematic diagram of pre-miR-21 wild type and mutant sequences (right panel: WT—SEQ ID NO: 134; M1—SEQ ID NO: 135; M2—SEQ ID NO: 136; M3—SEQ ID NO: 137; 5′ mut—SEQ ID NO: 138; Loop mut—SEQ ID NO: 139). Brackets indicate mature miRNA sequence and broken-lined boxes indicate the R-SBE sequence from pre-miR-21. Shaded sequences indicate nucleotides which are mutated. Predicted secondary structures of wild type and mutant pre-miR-21 (left panel: WT—SEQ ID NO: 128; M1—SEQ ID NO: 129; M2—SEQ ID NO: 130; M3—SEQ ID NO: 131; 5′ mut—SEQ ID NO: 132; Loop mut—SEQ ID NO: 133). Solid lines and circles indicate the SBE-sequence and the mutated residues, respectively. B. Mouse C3H10T1/2 cells were transfected with different pri-miR-21 expression constructs, followed by treatment with or without 3 nM BMP4 for 2 hr and subjected to qRT-PCR analysis using mature miR-21, pri-miR-21 or pre-miR-21 primers. Expression of mature miRNA was normalized to U6 snRNA. Expression of pri- and pre-miR-21 was normalized to GAPDH. Induction of WT, 5′ mut, and Loop mut by BMP4 is statistically significant. (*P<0.05) C. Cos7 cells were transfected with different pri-miR-21 expression constructs along with Flag-Smad1, Flag-Drosha or Flag-DGCR8 expression constructs, followed by BMP4 stimulation for 24 hr. RNA-IP assay was performed by immunoprecipitaion of RNA fragments with anti-Flag antibody, followed by PCR amplification with a set of primers for pri-miR-21 which specifically recognize exogenously expressed pri-miR-21. Expression of pri- or pre-miR-21 was examined by qRT-PCR analysis using pri-miR-21 or pre-miR-21 primers. Results were normalized to GAPDH. (*P<0.05)

FIG. 35 shows a direct association of Smad MH1 domain and the R-SBE A. In vitro transcribed wild type pri-miR-21 were mixed with recombinant GST alone or GST-Smad1 full length (FL), GST-Smad1(MH1), GST-Smad1(MH2), or GST-Smad5(FL) fusion proteins. Pri-miR-21 interacting with GST fusion proteins were precipitated and subjected to qRT-PCR analysis. Results are presented as fold-enrichment over the amount precipitated with GST alone. (*P<0.01, **P<0.05). B. In vitro transcribed wild type or mutant pri-miR-21 were mixed with recombinant GST alone or GST-Smad1(MH1). Pri-miR-21 interacting with GST fusion proteins were precipitated and subjected to qRT-PCR analysis. Results are presented as fold-enrichment over the amount precipitated with GST alone. (*P<0.01, **P<0.05). C. Nuclear extracts of Cos7 cells treated with BMP4 for 2 hr were mixed with equal amounts of in vitro transcribed, agarose bead-conjugated-pri-miR-21 wild type (WT) or mutants (bottom panel). Proteins affinity purified by pri-miR-21 were separated by SDS-PAGE and the presence of Smad1 or RNA helicase p68 was evaluated by immunoblot with anti-Smad1 or anti-p68 antibody. D. In vitro transcribed wild type pri-miR-21 conjugated to agarose beads were mixed with nuclear extract from Cos7 cells treated with BMP4 in the presence of 10-fold excess amount of pri-miR-21 with wild type (WT), Loop mut or R-SBE M3 RNAs (bottom panel). Proteins associated with pri-miR-21 were separated by SDS-PAGE and the amount of Smad1 was examined by immunoblot with anti-Smad1 antibody. Relative amount of Smad1 binding to pri-miR-21 was quantitated and shown in bottom panel. E. Synthetic RNA duplexes (miR-21 [top strand: SEQ ID NO: 73; bottom strand: SEQ ID NO: 140] or cel-miR-67 [top strand: SEQ ID NO: 125; bottom strand: SEQ ID NO: 141]) were mixed with recombinant GST alone or GST-Smad1(FL), GST-Smad1(MH1), GST-Smad1(MH2), GST-Smad3(FL), or GST-Smad4(FL) fusion proteins. Results are presented as fold-enrichment over the amount of RNA duplex precipitated with GST alone. F. 3-fold or 30-fold molar excess of DNA oligonucleotides with SBE1 or SBE2 (top panel), or 10-fold molar excess of in vitro transcribed R-SBE M3 RNA (control) were added during Smad1 pull-down assay using in vitro transcribed pri-miR-21(WT) conjugated to agarose beads [SBE-1: top strand—SEQ ID NO: 126; bottom strand—SEQ ID NO: 142; SBE-2: top strand—SEQ ID NO: 127; bottom strand—SEQ ID NO: 143]. The amount of Smad1 pulled down with pri-miR-21 was examined by immunoblot analysis with anti-Smad1 antibody (middle panel). Relative amount of Smad1 bound to pri-miR-21 was quantitated and presented (bottom panel).

FIG. 36 shows that introduction of R-SBE is sufficient for the TGFβ-regulated processing of pri-miRNA. A. Schematic diagram of pre-miRNA of cel-miR-84 wild type and mutant sequences (left panel; cel-mir-84(WT)—SEQ ID NO: 144; cel-mir-84(M1)—SEQ ID NO: 145; cel-mir-84(M2)—SEQ ID NO: 146; cel-mir-84(M3)—SEQ ID NO: 147). Brackets denote mature cel-miR-84 sequence and broken-lined boxes denotes the location of the introduced R-SBE sequence. Mouse C3H10T1/2 cells were transfected with the indicated pri-cel-miR-84 or pri-miR-21 expression constructs, followed by treatment with or without 3 nM BMP4 for 2 hr and subjected to qRT-PCR analysis using cel-miR-84 pri-miRNA or pre-miRNA primers (right panel). Expression of pri- and pre-miRNA was normalized to GAPDH. Induction of pre-miR-21 serves as a positive control for BMP4-regulated processing. B. Schematic diagram of the conserved sequence motif present in R-SBE containing miRNAs, indicating the average location of R-SBE in regulated miRNA. Smad bound to R-SBE provides a platform for a recruitment of Drosha and DGCR8.

FIG. 37 depicts a time course expression of pre-miRNAs in PASMCs and MDA-MB-468 cells after TGFβ or BMP4 treatment. A. A time-course expression of pre-miR-21, -23b, -25, -105, -199a, -215, or -509 was examined by qRT-PCR in PASMCs stimulated with 3 nM BMP4 (left panel) or 400 pM TGFβ1 (right panel). B. A time-course expression of pre-miR-21, -105, -421, -215, or -509 was examined by aRT-PCR in MDA-MB-468 cells stimulated with 3 nM BMP4 (left panel) or 400 pM TGFβ1 (right panel).

FIG. 38 shows that induction of pre-miRNAs by BMP4 is post-transcriptional. HPASMCs were treated with 10 μg/ml α-amanitin with or without 3 nM BMP4 for 5 hr and subjected to qRT-PCR analysis using primers specifically detect pre-miRNAs or Id3 primers normalized to GAPDH. Fold change in levels of pre-miRNAs in the BMP4 treated cells in comparison with untreated cells was presented. *P<0.05 (compared to no treatment).

FIG. 39 depicts a downregulation of Smad1/5 proteins by siRNA. PASMCs were transfected with non-targeting control siRNA (si-control) or mixture of siRNAs for Smad1 and Smad (si-Smads). Twenty-four hr afer transfection, cells were treated with BMP4 for 2 hr, and subjected to immunoblotting analysis using antibodies or anti-GAPDH antibody for loading control.

FIG. 40 shows partially purified GST-Smad fusion proteins. Recombinant GST alone, GST-Smad1 (FL, MH, or MH2), GST-Smad3(FL), or GST-Smad4(FL) fusion proteins are shown by Coomassie Blue staining of the SDS-PAGE gel.

FIG. 41 shows stem-loop sequences of pre-miRNAs that are regulated by TGF and BMP4. Mature miRNA sequences are denoted by brackets. The R-SBE sequences are shaded. miRNAs and corresponding sequence identifiers are as follows: miR-21—SEQ ID NO: 148; miR-199a-1—SEQ ID NO: 149; miR-199a-2—SEQ ID NO: 150; to miR-105—SEQ ID NO: 151; miR-215—SEQ ID NO: 152; miR-421—SEQ ID NO: 153; miR-509-1—SEQ ID NO: 154; miR-509-2—SEQ ID NO: 155; miR-509-3—SEQ ID NO: 156; miR-600—SEQ ID NO: 157.

### DETAILED DESCRIPTION

The invention in some aspects relates to compounds and compositions useful for modulating (activating or inhibiting) the TGF-β/BMP signaling pathway. The TGF-13/BMP signaling pathway comprises the transforming growth factor beta (TGF-β) superfamily that is a large family of structurally related cell regulatory proteins that was named after its first member, TGF-β1. Many proteins have since been described as members of the TGF-β superfamily in a variety of species, including invertebrates as well as vertebrates and categorized into 23 distinct gene types that fall into four major subfamilies: the decapentaplegic-Vg-related (DVR) related subfamily (including the bone morphogenetic proteins and the growth differentiation factors), the activin/inhibin subfamily, the TGF-β subfamily, and a subfamily encompassing various divergent members. These molecules play fundamental roles in the regulation of basic biological processes such as growth, development, tissue homeostasis and regulation of the immune system. They interact with a conserved family of cell surface serine/threonine-specific protein kinase receptors, and generate intracellular signals using a conserved family of proteins called SMADs, which are a class of proteins that modulate the activity of transforming growth factor beta ligands. The SMAD's form complexes, often with other SMAD's, enter the nucleus and serve as transcription factors. There are three classes of SMAD: receptor-specific SMADs (rSMAD) which include SMAD1, SMAD2, SMAD3, SMAD5 and SMAD8, common-mediator Smad (co-SMAD) which include only SMAD4, and antagonistic or inhibitory Smads(1-SMAD) which include SMAD6 and SMAD7.

It was discovered according to the invention that induction of a TGF β-BMP phenotype, such as a contractile phenotype in vascular smooth muscle cells (VSMCs), by TGF β and BMPs signaling is mediated by miRNAs such as miR-21. As shown in the specific examples below, miR-21 downregulates Programmed Cell Death 4 (PDCD4), which in turn acts as a negative regulator of smooth muscle contractile genes. Surprisingly, TGF β/BMP signaling promotes a rapid increase in expression of mature miR-21 and other miRNAs through a post-transcriptional step, promoting the processing of primary transcripts of miR-21 (pri-miR-21) into precursor miR-21 (pre-miR-21) by the Drosha complex. It was also discovered that TGF β- and BMP-specific Smad signal transducers are recruited to pri-miR-21 in a complex with the RNA helicase p68, a component of the Drosha microprocessor complex. The shared cofactor Smad4 is not required for this process. Thus, regulation of miRNA biogenesis by ligand-specific Smad proteins is important for control of TGF β-BMP signaling, such as producing a VSMC phenotype and for Smad4-independent responses mediated by the TGF β/BMP signaling pathways.

As used herein, a microRNA (miRNA) is an oligonucleotide that inhibits expression of one or more target mRNAs. Unless otherwise indicated, as used herein, the term miRNA encompasses primary miRNA (a pri-miRNA), a pre-miRNA, and a mature miRNA. It will be understood that a pri-miRNA may have a 5′ cap and a poly-A tail, and may be processed into a short stem-loop structure called a pre-miRNA. Similarly, it will be understood that a pre-miRNA may be further processed into a mature miRNA. Mature miRNAs comprise a seed sequence that is at least partially complementary to one or more target mRNA molecules, and function to down-regulate expression of the target mRNAs. In some embodiments, miRNAs are single-stranded RNA molecules of about 19-27 nucleotides in length, which regulate gene expression. In some embodiments, miRNAs are encoded by genes that are transcribed from DNA and processed from pri-miRNA to short stem-loop structures called pre-miRNA and finally to mature miRNA. Typically, an miRNA comprises a seed sequence that is at least partially complementary with a sequence of a target mRNA, e.g., a sequence in the 3′ untranslated regions (UTR) of a target miRNA. mRNAs typically effect their gene regulatory activity through sequence-specific interactions with the 3′ UTR of target mRNAs.

The invention, in some aspects, relates to the discovery of a class of microRNA (TGF MicroRNAs) that is responsive to TGF-β/BMP signaling. The invention, in some aspects, relates to TGF-β/BMP/miR pathway modulators (activators or inhibitors), which activate or inhibit expression of TGF MicroRNAs. In general TGF-β/BMP/miR pathway activators of this invention include agents which promote miRNA processing and accumulation in a cell. Whereas, TGF-β/BMP/miR pathway inhibitors of this invention include agents that block miR expression or processing.

In some aspects, the oligonucleotides of the invention are based on the discovery that SMAD binds to and regulates the processing of miRNAs, which are referred to herein as “TGF microRNAs” or “TGF miRNAs”. TGF microRNA are any miRNAs that are regulated by the TGF-β/BMP signaling pathway. The TGF microRNAs may be upregulated or down regulated in response to TGF-β/BMP signaling. In some embodiments the TGF microRNAs share a common motif, a CAGRN motif, which may be equivalently represented as, CAG(A/G)(C/A/G/U). The CAGRN motif may also be referred to herein as a RNA SMAD Binding Element (R-SBE) or a common motif. In certain embodiments the TGF microRNAs share a common motif, a CAGAC. In some embodiments the TGF microRNAs share as a common motif, a CAGAB sequence, which may be equivalently represented as, CAGA(C/G/U). Unless otherwise specified C, A, G, and U refer to naturally occurring nucleosides, synthetic nucleosides, or modified versions thereof. In other embodiments the start of the common motif is located at least 4 nucleotides from the loop in the stem portion of the pre-miRNA. In other embodiments the start of the common motif is located 5, 6, 7, 8, 9, or 10 nucleotides from the loop in the stem portion of the pre-miRNA. In some embodiments, the start of the common motif is about 4 to about 12 nucleotides away from the Drosha cleavage site. In some embodiments, the common motif is up to about 15 nucleotides away from the Drosha cleavage site. In some embodiments, the common motif is 1, 2, 3, 4, 5, 6, 7, 8, 9, 10 or more nucleotides away from 5′ end of the mature miRNA. Thus, the TGF microRNAs may be a group of microRNAs having the common motif that binds to SMAD and that is positioned within the binding region of a primary miRNA microRNA processing complex (Drosha complex). Enhancement of SMAD binding to common motif (e.g., by overexpression of SMAD or a function fragment thereof) may promote processing of the miRNA by SMAD In some embodiments, inhibition of SMAD binding to the common motif inhibits processing of the miRNA.

Thus, the invention encompasses compounds and compositions of TGF-β/BMP/miR pathway activators or inhibitors as well as therapeutic, research and diagnostic methods of using such compounds.

A TGF-β/BMP/miR pathway activator of this invention is an agent which promotes TGF miRNA processing and/or accumulation in a cell. For instance, these molecules include exogenous miRNAs corresponding to TGF microRNAs, vectors encoding TGF microRNAs, exogenous SMADs and fragments thereof that are active to promote processing, and vectors encoding SMADs.

An exogenous miRNA is an oligonucleotide that is a pri-miRNA, a pre-miRNA, or a miRNA that enhances (e.g., supplement) or restore the presence or function of an miRNA downregulated in disease. In some embodiments downregulation of miRNA is causally related to the disease. For example, an exogenous miRNA may be delivered to cells to supplement the expression of an miRNA that is reduced in a TGF-β/BMP Mediated Disorder to treat the disorder.

The exogenous miRNA, may have a sequence identical to an endogenous pri-miRNA, pre-miRNA, or miRNA. Alternatively the exogenous miRNA may have a sequence substantially similar to the sequence of an endogenous pri-miRNA, pre-miRNA, or miRNA such that the oligonucleotide is sufficiently complementary to at least one target mRNA of the miRNA and is capable of hybridizing with and inhibiting the target mRNA. An exogenous miRNA may be a synthetic miRNA. An exogenous miRNA may also be an miRNA comprising a heterologous CAGRN sequence. In some embodiments, an oligonucleotide sequence that is substantially similar to the sequence of an miRNA, is a sequence that is identical to the miRNA sequence at all but 1, 2, 3, 4, 5, 6, 7, 8, 9, 10, 11, 12, 13, 14, 15, 16, 17, 18, 19, 20 or more bases. In some embodiments, an oligonucleotide sequence that is substantially similar to the sequence of an endogenous pri-miRNA, pre-miRNA, or miRNA, is a sequence that is different than the miRNA sequence at all but up to one base. In yet other embodiments the exogenous miRNA has at least 75% homology with an endogenous pri-miRNA, pre-miRNA, or miRNA. In other embodiments the homology is greater than 80%, 85%, 90%, 95%, 96%, 97%, 98%, or 99%.

Any one of a number of oligonucleotides types known in the art and/or disclosed herein (e.g., siRNA, miRNA, or shRNA) can be used for supplementing miRNA expression (activity). In some embodiments, an miRNA is supplemented by delivering an siRNA having a sequence that comprises the sequence, or a substantially similar sequence, of the miRNA.

The TGF-β/BMP/miR pathway activators also include exogenous SMADs and functional fragments thereof. The activators include receptor-specific SMADs (rSMAD) which include SMAD1, SMAD2, SMAD3, SMAD5 and SMAD8. A functional fragment of SMAD is a portion of the full length protein that can activate TGF-β/BMP mediated miRNA processing.

An exogenous SMAD or fragment thereof may have a sequence identical to an endogenous SMAD or it may have a sequence identical to a fragment of an endogenous SMAD. Alternatively the exogenous SMAD may include one or more modifications. In some embodiments, modification of the sequence of the coding region or fragment thereof results in a variant of SMAD. The skilled artisan will realize that conservative amino acid substitutions may be made in SMADs to provide functionally equivalent variants, or homologs, i.e., the variants retain the functional capabilities of the SMAD (e.g., TGF miRNA processing). In some aspects the invention embraces sequence alterations that result in one or more conservative amino acid substitution of SMADs. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of SMAD include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the SMADs disclosed herein and retain the miRNA processing properties.

TGF-β/BMP/miR pathway activators also include miRNA expression vectors and SMAD expression vectors. For instance, the miRNA can be supplemented by delivering miRNAs encoded by shRNA vectors. Such technologies for delivering exogenous microRNAs to cells are well known in the art. For example, the shRNA-based vectors encoding nef/U3 miRNAs produced in HIV-1-infected cells have been used to inhibit both Nef function and HIV-1 virulence through the RNAi pathway (Omoto S et al. Retrovirology. 2004 Dec. 15; 1:44). An miRNA expression vector is a vector that includes the elements necessary to express and includes nucleic acids encoding for an exogenous miRNA as described above, thus including pri-miRNA, pre-miRNA, and miRNA. A SMAD expression vector is a vector that includes the elements necessary to express and includes nucleic acids encoding for an exogenous SMADs or functional fragments thereof as described above. Other details relating to examples of vectors are provided below.

In some embodiments the TGF-β/BMP/miR pathway activator is one that has not previously been indicated for the treatment of a therapeutic disorder described herein. A “TGF-β/BMP/miR pathway activator is one that has not previously been indicated for the treatment of a therapeutic disorder” as used herein refers to a compound that had not, prior to the invention, been proposed for the treatment of the disease for which it is now, based on the discoveries of the invention, being used. For instance in this embodiment, a drug which had previously been proposed for the treatment of a bone disease would not fall within the scope of this particular embodiment even if it is a TGF-β/BMP/miR pathway activator.

A “TGF-β/BMP/miR Inhibitor” of this invention is an agent that blocks TGF miRNA expression and/or processing. For instance, these molecules include but are not limited to TGF microRNA specific antisense, TGF microRNA sponges, TGF microRNA oligonucleotides (double-stranded, hairpin, short oligonucleotides) that inhibit miRNA interaction with a Drosha complex, and SMAD inhibitors.

An miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of an miRNA is accomplished by inhibiting the miRNA activity in cells through any one of a variety of methods. For example, blocking the activity of an miRNA can be accomplished by hybridization with a TGF microRNA antisense oligonucleotide that is complementary, or substantially complementary to, the miRNA, thereby blocking interaction of the miRNA with its target mRNA. A TGF microRNA antisense oligonucleotide that is substantially complementary to an miRNA is an oligonucleotide that is capable of hybridizing with an miRNA, thereby blocking the miRNA's activity. The TGF microRNA antisense oligonucleotide may have perfect complementarity with it's miRNA target. Alternatively it may have less than perfect complementarity as long as it is substantially complementary to an miRNA target such that it reduces the activity of the miRNA. A TGF microRNA antisense oligonucleotide that is substantially complementary to an miRNA may be complementary with the miRNA at all but 1, 2, 3, 4, 5, 6, 7, 8, 9, 10, 11, 12, 13, 14, 15, 16, 17, 18, 19, 20 or more nucleotides. In some embodiments, an oligonucleotide sequence that is substantially complementary to an miRNA, is an oligonucleotide sequence that has at least 75%, 80%, 85%, 90%, 95%, 96%, 97%, 98%, or 99% homology with a sequence that is perfectly complementary to the target miRNA. Antisense oligonucleotides, including chemically modified antisense oligonucleotides—such as 2′ O-methyl, locked nucleic acid (LNA)—inhibit miRNA activity by hybridization with guide strands of mature miRNAs, thereby blocking their interactions with target mRNAs (Naguibneva, I. et al. Nat. Cell Biol. 8, 278-284 (2006), Hutvagner G et al. PLoS Biol. 2, e98 (2004), Orom, U. A., et al. Gene 372, 137-141 (2006), Davis, S, Nucleic Acid Res. 34, 2294-2304 (2006)). ‘Antagomirs’ are phosphorothioate modified oligonucleotides that can specifically block miRNA in vivo (Kurtzfeldt, J. et al. Nature 438, 685-689 (2005)).

MicroRNA inhibitors, termed miRNA sponges, can be expressed in cells from transgenes (Ebert, M. S, Nature Methods, Epub Aug. 12, 2007). The inhibitors of the invention encompass TGF microRNA sponges. These TGF microRNA sponges specifically inhibit TGF miRNAs through a complementary heptameric seed sequence. An entire family of miRNAs can be silenced using a single sponge sequence. Other methods for silencing miRNA function (derepression of miRNA targets) in cells will be apparent to one of ordinary skill in the art.

SMAD inhibitors, as used herein, refers to compounds that interfere with the TGFβ/BMP signal transduction functions of SMAD, specifically by interfering with SMAD-miR interactions, and/or with SMAD p-68 interactions. In some embodiments, antagonistic or inhibitory Smads (1-SMAD) which include SMAD6 and SMAD7 are useful as SMAD inhibitors. In other embodiments, functional fragments of receptor-specific SMAD (e.g., MH1 or MH2 domain fragments) that block miRNA processing are useful as SMAD inhibitors. As disclosed herein, the MH2 domain of SMAD (e.g., SMAD1, SMAD5) interacts with p68. Thus, in some embodiments, SMAD fragments comprising all or a portion of the MH2 domain and that bind p68 are useful a competitive inhibitors of SMAD binding to p68. In some embodiments, inhibition of SMAD binding to p68 inhibits processing of primary miRNA (e.g., TGF miRNAs). As disclosed herein, SMAD fragments comprising MH1 and/or MH2 domain and that bind miRNA (e.g., a TGF miRNA such as miR21) are useful as SMAD inhibitors. Thus, in some embodiments, SMAD fragments comprising all or a portion of the MH1 and/or MH2 domain and that bind miRNA are useful a competitive inhibitors of SMAD binding to miRNA. In some embodiments, inhibition of SMAD binding to miRNA inhibits processing of primary miRNA (e.g., TGF miRNAs). In other embodiments binding peptides such as antibodies, diabodies, antibody fragments are SMAD inhibitors.

In some embodiments the TGF-β/BMP/miR pathway inhibitor is one that has not previously been indicated for the treatment of a therapeutic disorder described herein, for instance cancer. A “TGF-β/BMP/miR pathway inhibitor is one that has not previously been indicated for the treatment of a therapeutic disorder” as used herein refers to a compound that had not, prior to the invention, been proposed for the treatment of the disease for which it is now, based on the discoveries of the invention, being used. For instance in this embodiment, a drug which had previously been proposed for the treatment of cancer would not fall within the scope of this particular embodiment even if it is a TGF-β/BMP/miR pathway inhibitor.

TGF microRNA oligonucleotides are also TGF-β/BMP pathway inhibitors of the invention. In some aspects, the oligonucleotides of the invention are based on the discovery that SMAD binds to and regulates the processing of TGF microRNAs having a core motif. For instance, one TGF microRNA core motif is a CAGRN motif. Inhibition of binding of SMAD protein to miRNA (i.e., primary miRNA) comprising a TGF microRNA core motif inhibits processing of the miRNA. Thus, the TGF microRNA oligonucleotides in some aspects of the invention, are CAGRN oligonucleotides. A CAGRN oligonucleotide of the invention is a hairpin, double- or single-stranded oligonucleotide that is a competitive inhibitor of SMAD binding to endogenous TGF microRNA and as such, that are thereby capable of inhibiting the processing of the endogenous miRNAs. The CAGRN motif is identified herein as being present in a class of miRNAs (i.e., TGF microRNAs) that are regulated by the TGF-β/BMP signaling pathway. In some embodiments the CAGRN motif binds to SMAD and is positioned within the binding region of a primary miRNA microprocessor complex (Drosha complex). In other embodiments, inhibition of SMAD binding to the CAGRN motif inhibits processing of the miRNA.

The term “oligonucleotide” (also referred to interchangeably as nucleic acids and polynucleotide) refers to a molecule having multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the terms oligonucleotide refers to oligonucleotides having ribonucleotides deoxyribonucleotides, and combinations thereof. The term “oligonucleotide” may also include polynucleosides (i.e., a polynucleotide minus the phosphate) and any other organic base containing polymer. The compounds described herein may be isolated compounds.

An “isolated compound” generally refers to a compound which is separated from components which it is normally associated with in nature and/or a synthetic compounds. An isolated compound includes, for instance, isolated nucleic acids such as isolated oligonucleotides and isolated peptides. As used herein the term “isolated nucleic acid molecule” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a small percentage of the material in the cell in which it resides. Such a nucleic acid is isolated, however, as the term is used herein because it is readily manipulable by standard techniques known to those of ordinary skill in the art.

Thus, an “isolated oligonucleotide” generally refers to an oligonucleotide which is separated from components which it is normally associated with in nature and/or a synthetic oligonucleotide. As an example, an isolated oligonucleotide may be one which is separated from a cell, from a nucleus, from mitochondria or from chromatin. Nucleic acid molecules can be obtained from existing nucleic acid sources (e.g., genomic or cDNA), but are preferably synthetic (e.g., produced by nucleic acid synthesis).

The invention encompasses an isolated oligonucleotide and uses thereof. The isolated oligonucleotides may have the formula (SEQ ID NO: 1) 5′-(X¹)ᵢ C A G A C (X²)ⱼ-3′ or (SEQ ID NO: 2) 5′-(X¹)ᵢ G U C U G (X²)ⱼ-3′. These are single-stranded oligonucleotides wherein each of X¹ and X² is independently any nucleotide, wherein i and j independently represent at least one nucleotide, and wherein the isolated oligonucleotide has a length of from 20 to 30 nucleotides. In some cases, i and j independently represent from 1 to 20 nucleotides. In some cases, i and j independently represent from 5 to 16 nucleotides. These single stranded oligonucleotides may have a length of from 21 to 27 nucleotides. The oligonucleotides may also include at least one modified nucleotide and/or internucleotide bond such as those described in more detail below. In some instances the modified nucleotide may be an inosine or ribothymidine. A modified internucleotide bond may be a stabilized linkage, such as those described in more detail below including a phosphonoacetate, a phosphorothioate, a phosphorodithioate, a methylphosphonate, a methylphosphorothioate, a 2′-5′ linkage, a peptide linkage, and dephospho bridge.

The foregoing single-stranded oligonucleotides have a variety of uses. Preferably, the oligonucleotides are useful as TGFβ/BMP/miR pathway modulators. For example, in some cases they are useful as antisense oligonucleotides that inhibit the activity of miRNAs (e.g., TGF miRNAs). In other cases they are useful as antisense oligonucleotides that inhibit the activity of mRNAs (e.g., mRNA targets of TGF miRNAs). Other uses will be apparent to the skilled artisan.

The isolated oligonucleotides may also have a substantially double-stranded portion having the nucleotide sequence CAGRN. Preferably these isolated oligonucleotides inhibit the binding of a receptor-specific SMAD (rSMAD) protein to an miRNA. The isolated oligonucleotides in this aspect of the invention may have a sequence of a primary miRNA of miR-21, miR-199a, miR-105, miR-509-1(5p), miR-421, or miR-600r. Alternatively these isolated oligonucleotides may not have a sequence of a primary miRNA of miR-21, miR-199a, miR-105, miR-509-1(5p), miR-421, or miR-600r.

As used herein, “substantially double-stranded portion” is a portion of a oligonucleotide (typically at least 5 nucleotides in length) having a strand with three or more (typically contiguous) nucleotides engaged in complementary hydrogen bond pairs with three or more (typically contiguous) nucleotides of a portion of another strand. The complementary base pairing in the substantially double stranded portion may be intermolecular or intermolecular. That is, the base pairing may be between two separate oligonucleotides (e.g., double stranded oligonucleotides), or within a single oligonucleotide (e.g., hairpin oligonucleotides). Typically, the double stranded portion consists of at least 5 nucleotides within which at least three contiguous nucleotides (i.e., three contiguous bases pairs) are engaged in complementary hydrogen bonds. In a preferred embodiment, the substantially double stranded portion consists of at least 5 bases (e.g., CAGAC) having only one nucleotide that is not engaged in a complementary base pair. Thus, in some cases, the substantially double-stranded portion having the nucleotide sequence CAGRN has one mismatch in the nucleotide sequence CAGRN. In another preferred embodiment, the substantially double stranded portion consists of at least 5 bases (e.g., CAGAC) having all 5 nucleotides engaged in complementary base pairs (i.e., entirely double stranded). Thus, in some cases, the substantially double-stranded portion having the nucleotide sequence CAGRN is entirely double-stranded in the nucleotide sequence CAGRN. The substantially double-stranded portion having the nucleotide sequence CAGRN may bind to SMAD (e.g., SMAD1, SMAD 2, SMAD 3, SMAD 5, and SMAD 8) and functional fragments thereof (e.g., MH1 domain fragments).

In some aspects, where the invention relates to an isolated oligonucleotide comprising a substantially double-stranded portion having the nucleotide sequence CAGAC, wherein the isolated oligonucleotide inhibits the binding of a receptor-specific SMAD (rSMAD) protein to an miRNA, the rSMAD protein is selected from SMAD1, SMAD2, SMAD3, SMAD5 and SMAD8.

The oligonucleotides may have a formula selected from:

(SEQ ID NO: 3)
5′- (X¹)₍ᵢ₊ⱼ₎ C A G A C (X²)ₖ G U C U G
(X³)₍ᵢ₊ₘ₎ -3′,
(SEQ ID NO: 4)
5′- (X¹)₍ᵢ₊ⱼ₎ G U C U G (X²)ₖ C A G A C
(X³)₍ᵢ₊ₘ₎ -3′
and
(SEQ ID NO: 5)
5′- (X¹)₍ᵢ₊ⱼ₎ C A G A C (X²)ₖ G U C G
(X³)₍ᵢ₊ₘ₎ -3′,

These are hairpin oligonucleotides wherein each of the X¹, X², and X³ is independently any nucleotide, wherein i represents at least one nucleotide, wherein k represents at least one nucleotide, and wherein j and m independently represent zero or more overhang nucleotides, and wherein (X²)ₖ forms a loop structure. In some embodiments, i and/or k represents up to about 10, 20, 30, 40, 50, 60, 70, 80, 90, or 100 or more nucleotides. In some embodiments, i represents about 1 to 45 nucleotides. In some embodiments, i represent 5 to 26 nucleotides. In some embodiments, k represents about up to about 10, 20, 30, 40, 50, 60, 70, 80, 90, or 100 or more nucleotides. In some embodiments, k represent 26 to 35 nucleotides. In some embodiments, j and m independently represent 0, 1, 2, 3, 4, 5, or 6 overhang nucleotides. As used herein overhang nucleotides are nucleotides at the 5′ or 3′ end of a oligonucleotide (either double stranded or hairpin oligonucleotides) that not engaged in complementary hydrogen bonding. In some embodiments, (X³)₍ᵢ₊ₘ₎ contains up to 0, 1, 2, 3, 4, 5, 6, 7, 8, 9, or 10 or more mismatches with the reverse complement of (X¹)₍ᵢ₊ⱼ₎. Mismatches can be substitutions, deletions, or additions. Exemplary, hairpin oligonucleotide of the foregoing formulas, are shown in Example 9 and have the sequence as set forth in SEQ ID NO: 73, SEQ ID NO: 74, SEQ ID NO: 75, SEQ ID NO: 76, SEQ ID NO: 77, or SEQ ID NO: 80.

The foregoing hairpin oligonucleotides, have a variety of uses. For example, in some embodiments, the hairpin oligonucleotides inhibit the binding of a receptor-specific SMAD (rSMAD) protein to a primary miRNA, and thereby inhibit processing of the primary miRNA and, consequently, expression of the mature miRNA. In another example, the hairpin oligonucleotides are exogenous shRNA, shRNA-miR or miRNA that can enhance (e.g., supplement an endogenous miRNA) or restore the expression of a mature miRNA.

The oligonucleotides that are useful for inhibiting miRNA expression may have a double stranded oligonucleotide formula selected from:

(SEQ ID NO: 6)
5′- (X¹)₍ᵢ₊ⱼ₎ C A G A C (X³)₍ₖ₊ₘ₎ -3′
(SEQ ID NO: 7)
3′- (X²)₍ᵢ₊ₙ₎ G U C U G (X⁴)₍ₖ₊ₚ₎ -5′,
(SEQ ID NO: 6)
5′- (X¹)₍ᵢ₊ⱼ₎ C A G A C (X³)₍ₖ₊ₘ₎ -3′
(SEQ ID NO: 8)
3′- (X²)₍ᵢ₊ₙ₎ G - C U G (X⁴)₍ₖ₊ₚ₎ -5′,
and
(SEQ ID NO: 6)
5′- (X¹)₍ᵢ₊ⱼ₎ C A G A C (X³)₍ₖ₊ₘ₎ -3′
(SEQ ID NO: 9)
3′- (X²)₍ᵢ₊ₙ₎ - U C U G (X⁴)₍ₖ₊ₚ₎ -5′,

These are hairpin oligonucleotides wherein each of X¹, X², X³, and X⁴, is independently any nucleotide, wherein i and k independently represent at least one nucleotide, and wherein j, n, m and p independently represent zero or more overhang nucleotides. In some embodiments, i and/or k independently represent 1 to 20 nucleotide. In some embodiments, i represents 5 to 16 nucleotides. In some embodiments, k represents 6 to 13 nucleotides. In some embodiments, j, n, m and p independently represent from 0 to 6 overhang nucleotides. In some embodiments, each strand of the isolated double-stranded oligonucleotide independently has a length of from 20 to 30 nucleotides. In some embodiments, each strand of the isolated oligonucleotide independently has a length of from 21 to 27 nucleotides. In some embodiments, (X²)₍ᵢ₊ₙ₎ is reverse complementary to (X¹)₍ᵢ₊ⱼ₎ at about 1 to i nucleotide positions. In some embodiments, (X¹)₍ᵢ₊ⱼ₎ contains up to 0, 1, 2, 3, 4, 5, 6, 7, 8, 9, or 10 or more mismatches with the reverse complement of (X²)₍ᵢ₊ₙ₎. In some embodiments, (X⁴)₍ₖ₊ₚ₎ is reverse complementary to (X³)₍ₖ₊ₘ₎ at about 1 to i nucleotide positions. In some embodiments, (X³)₍ₖ₊ₘ₎ contains up to 0, 1, 2, 3, 4, 5, 6, 7, 8, 9, or 10 or more mismatches with the reverse complement of (X⁴)₍ₖ₊ₚ₎.

Exemplary, double-stranded oligonucleotides of the foregoing formulas, have the sequence as set forth in SEQ ID NO: 73, SEQ ID NO: 74, SEQ ID NO: 75, SEQ ID NO: 76, SEQ ID NO: 77, or SEQ ID NO: 80. The foregoing hairpin oligonucleotides, have a variety of uses. For example, in some embodiments, the double-stranded oligonucleotide inhibit the binding of a receptor-specific SMAD (rSMAD) protein to a primary miRNA, and thereby inhibits processing of the primary miRNA and, consequently, express of the mature miRNA. In another example, the double-stranded oligonucleotides are exogenous siRNA or miRNA that can enhance (e.g., supplement an endogenous miRNA) or restore the expression of an mature miRNA.

The oligonucleotides disclosed herein can be synthesized de novo using any of a number of procedures well known in the art. For example, the β-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid Res. 14:5399-5407, 1986; Garegg et al., Tet. Let. 27:4055-4058, 1986, Gaffney et al., Tet. Let. 29:2619-2622, 1988). These chemistries can be performed by a variety of automated nucleic acid synthesizers available in the market. These oligonucleotides are referred to as synthetic oligonucleotides.

The term “oligonucleotide” also encompasses nucleic acids or oligonucleotides with substitutions or modifications, such as in the bases and/or sugars. For example, they include nucleic acids having backbone sugars that are covalently attached to low molecular weight organic groups other than a hydroxyl group at the 2′ position and other than a phosphate group or hydroxy group at the 5′ position. Thus modified nucleic acids may include a 2′-O-alkylated ribose group. In addition, modified nucleic acids may include sugars such as arabinose or 2′-fluoroarabinose instead of ribose. Thus the nucleic acids may be heterogeneous in backbone composition thereby containing any possible combination of polymer units linked together such as peptide-nucleic acids (which have an amino acid backbone with nucleic acid bases). Other examples are described in more detail below.

The oligonucleotides of the instant invention can encompass various chemical modifications and substitutions, in comparison to natural RNA and DNA, involving a phosphodiester internucleoside bridge, a β-D-ribose unit and/or a natural nucleoside base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-129; and Hunziker J et al. (1995) Mod Synth Methods 7:331-417. An oligonucleotide according to the invention may have one or more modifications, wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

For example, the oligonucleotides may comprise one or more modifications and wherein each modification is independently selected from:

a) the replacement of a phosphodiester internucleoside bridge located at the 3′ and/or the 5′ end of a nucleoside by a modified internucleoside bridge,
b) the replacement of phosphodiester bridge located at the 3′ and/or the 5′ end of a nucleoside by a dephospho bridge,
c) the replacement of a sugar phosphate unit from the sugar phosphate backbone by another unit,
d) the replacement of a β-D-ribose unit by a modified sugar unit, and
e) the replacement of a natural nucleoside base by a modified nucleoside base.

More detailed examples for the chemical modification of an oligonucleotide are disclosed herein.

The oligonucleotides may include modified internucleotide linkages, such as those described in a or b above. These modified linkages may be partially resistant to degradation (e.g., are stabilized). A stabilized oligonucleotide molecule is an oligonucleotide that is relatively resistant to in vivo degradation (e.g. via an exo- or endo-nuclease) resulting form such modifications. Oligonucleotides having phosphorothioate linkages, in some embodiments, may provide maximal activity and protect the oligonucleotide from degradation by intracellular exo- and endo-nucleases. Typically oligonucleotides have phosphorothioate or other stabilized bonds located at the 5′ and 3′ portions of the molecule. In some embodiments, the entire oligonucleotide is fully stabilized.

A phosphodiester internucleoside bridge located at the 3′ and/or the 5′ end of a nucleoside can be replaced by a modified internucleoside bridge, wherein the modified internucleoside bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)—O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleoside by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a 13-D-ribose and phosphodiester internucleoside bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA”; as described for example, in Nielsen P E et al. (1994) Bioconjug Chem 5:3-7), that is, e.g., the replacement by a PNA backbone unit, e.g., by 2-aminoethylglycine. The oligonucleotide may have other carbohydrate backbone modifications and replacements, such as peptide nucleic acids with phosphate groups (PHONA), locked nucleic acids (LNA), and oligonucleotides having backbone sections with alkyl linkers or amino linkers. The alkyl linker may be branched or unbranched, substituted or unsubstituted, and chirally pure or a racemic mixture.

A β-ribose unit or a β-D-2′-deoxyribose unit can be replaced by a modified sugar unit, wherein the modified sugar unit is for example selected from (3-D-ribose, β-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′—NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some embodiments the sugar is 2′-O-methylribose, particularly for one or both nucleotides linked by a phosphodiester or phosphodiester-like internucleoside linkage.

A modified base is any base which is chemically distinct from the naturally occurring bases typically found in DNA and RNA such as T, C, G, A, and U, but which share basic chemical structures with these naturally occurring bases. The modified nucleoside base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleosides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleoside bases. This list is meant to be exemplary and is not to be interpreted to be limiting.

The oligonucleotides of the instant invention may include lipophilic nucleotide analogs. Preferred lipophilic nucleotide analogs are e.g. 5-chloro-uracil, 5-bromo-uracil, 5-iodo-uracil, 5-ethyl-uracil, 5-propyl-uracil, 2,4-difluoro-toluene, and 3-nitropyrrole.

The internucleotide linkages in the oligonucleotide may be non-stabilized or stabilized linkages (against nucleases), preferably phosphodiester (non stabilized), a phosphorothioate (stabilized) or another charged backbone. The chirality of a particular linkage may be random, of an Rp or Rs configuration.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann, E. and Peyman, A., Chem. Rev. 90:544, 1990; Goodchild, J., Bioconjugate Chem. 1:165, 1990).

In another aspect of the invention the modified oligonucleotides have a lipophilic moiety (e.g., lipid moiety). A “lipophilic moiety” as used herein is a lipophilic group covalently attached to an end or internal portion of the modified oligonucleotide. The lipophilic group in general can be a cholesteryl, a modified cholesteryl, a cholesterol derivative, a reduced cholesterol, a substituted cholesterol, cholestan, C₁₋₆ alkyl chain, a bile acid, cholic acid, taurocholic acid, deoxycholate, oleyl litocholic acid, oleoyl cholenic acid, a glycolipid, a phospholipid, a sphingolipid, an isoprenoid, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen. In certain embodiments the lipophilic moiety is chosen from cholesteryl, palmityl, and fatty acyl. In one embodiment the lipohilic moiety is cholesteryl.

In one embodiment the lipophilic group is attached to a 2′-position of a nucleotide of the modified oligonucleotide. A lipophilic group can alternatively or in addition be linked to the heterocyclic nucleobase of a nucleotide of the modified oligonucleotide. The lipophilic moiety can be covalently linked to the modified oligonucleotide via any suitable direct or indirect linkage. In one embodiment the linkage is direct and is an ester or an amide. In one embodiment the linkage is indirect and includes a spacer moiety, for example one or more abasic nucleotide residues, oligoethyleneglycol, such as triethyleneglycol (spacer 9) or hexaethylenegylcol (spacer 18), or an alkane-diol, such as butanediol.

The isolated oligonucleotides may also be conjugated to a Nuclear Localization Signal (NLS). As used herein, the term “nuclear localization signal” means an amino acid sequence known to, in vivo, direct a compound disposed in the cytoplasm of a cell across the nuclear membrane and into the nucleus of the cell. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The inclusion of a nuclear localization signal (NLS) as a delivery vehicle component is an aspect of the present invention. A representative nuclear localization signal is a peptide sequence that directs the compound to the nucleus of the cell in which the sequence is expressed. A nuclear localization signal is predominantly basic, can be positioned almost anywhere in a protein's amino acid sequence, generally comprises a short sequence of four amino acids (Agrawal, (1998) J. Biol. Chem. 273: 14731-37) to eight amino acids, and is typically rich in lysine and arginine residues (Magin et al., (2000) Virology 274: 11-16). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Leff. 461:229-34. Translocation is currently thought to involve nuclear pore proteins.

Nuclear localization signals appear at various points in the amino acid sequences of proteins. NLS's have been identified at the N-terminus, the C-terminus and in the central region of proteins. Thus, a selected sequence can serve as the functional component of a longer peptide sequence. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

In a preferred embodiment of the present invention, a nuclear localization signal be attached (conjugated) to the isolated nucleotide. The nuclear localization signal can be synthesized or excised from a larger sequence. As noted, a variety of nuclear localization signals are known and selection of an appropriate sequence can be made based on the known properties of these various sequences. Representative NLSs include monopartite sequences such as that from SV40 large T antigen and the c-myc proto-oncogene. Bipartite signals are characterized as a small cluster of basic residues positioned 10-12 residues N-terminal to a monopartite-like sequence. An example of a bipartite nuclear localization signal is that from nucleoplasmin. In some embodiments, a NLS selected from the following list may be conjugated to the oligonucleotide: SV40 large T Antigen: PKKKRKV (SEQ ID NO: 10); Nucleoplasmin: KRPAAIKKAGQAKKKK (SEQ ID NO: 11); CBP80: RRRHSDENDGGQPHKRRK (SEQ ID NO: 12); HIV-1 Rev: RQARRNRRRWE (SEQ ID NO: 13); HTLV-1 Rex: MPKTRRRPRRSQRKRPPT (SEQ ID NO: 14); hnRNP A: NQSSNFGPMKGGNFGGRSSGPYGGGGQYFAKPRNQGGY (SEQ ID NO: 15); c-myc PAAKRVKLD (SEQ ID NO: 16) and rpL23a: VHSHKKKKIRTSPTFTTPKTLRLRRQPKYPRKSAPRRNKLDHY (SEQ ID NO: 17).

In one embodiment of the invention, the nuclear localization signal comprises the motif K(K/R)X(K/R) (SEQ ID NO: 18). In a specific embodiment, the nuclear localization signal is KRXR (SEQ ID NO: 19), wherein X is any amino acid.

In some aspects, the invention relates to miRNAs comprising a heterologous substantially double-stranded portion comprising the nucleotide sequence CAGRN that promotes binding of a receptor-specific SMAD (rSMAD) protein to the miRNA, wherein R is A or G and N is A, G, C, or U. Typically, the heterologous substantially double-stranded portion comprising the nucleotide sequence CAGRN is incorporated into the stem loop of an miRNA that is not regulated by TGFβ/BMP signaling to render the miRNA responsive to TGFβ/BMP signaling. However, in some cases, the heterologous substantially double-stranded portion comprising the nucleotide sequence CAGRN is incorporated into the stem loop of an miRNA that is regulated by TGFβ/BMP signaling to enhance or augment the responsiveness of the miRNA to TGFβ/BMP signaling. Accordingly, the miRNA may or may not have a homologous substantially double-stranded portion having the nucleotide sequence CAGRN. It will be appreciated that the heterologous portion can be incorporated into the miRNA by any one of a variety of methods known in the art. For example, an miRNA having a heterologous portion can be synthesized directly. Alternatively, a heterologous portion can be incorporated into a miRNA using recombinant technology. Other approaches will be apparent to the skilled artisan. MiRNAs having a heterologous portions comprising the nucleotide sequence CAGRN may also comprise a seed sequence that targets a gene associated with a TGF-β/BMP mediated disorder, such as a fibroproliferative disorder, a cancer, or an autoimmune disease.

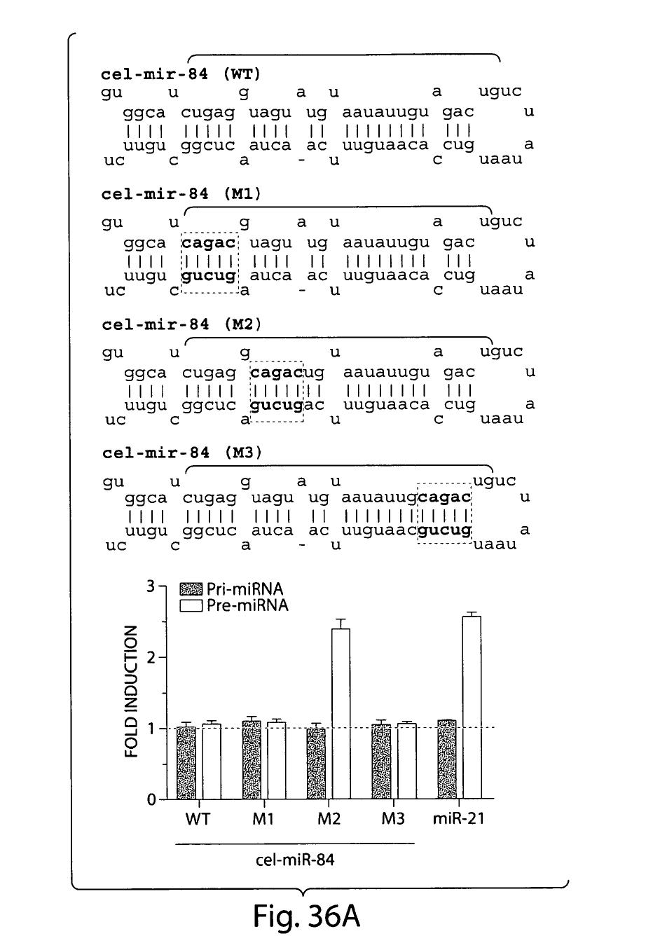

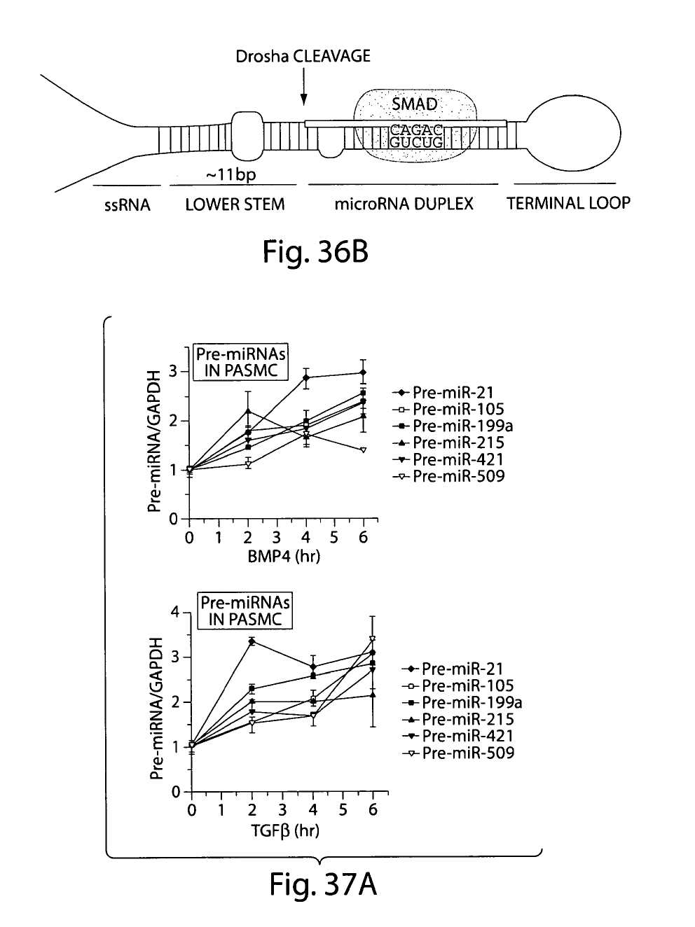

Synthetic (artificial) miRNAs may also be prepared that comprise a seed sequence, e.g., that targets a gene associated with a TGF-β/BMP mediated disorder, and a substantially double-stranded portion comprising the nucleotide sequence CAGRN that promotes binding of a receptor-specific SMAD (rSMAD) protein to the synthetic miRNA, wherein R is A or G and N is A, G, C, or U. Synthetic miRNAs may include a ssRNA region, a lower stem region, a Drosha cleavage site, a mature miRNA duplex region, which comprises the CAGRN and seed sequences, and a terminal loop region (See, FIG. 36B, for example). Typically, CAGRN and seed sequences do not overlap. Synthetic miRNAs may also be mature miRNAs consisting of a duplex region, which may contain one or more mismatches, that comprises the CAGRN and seed sequences. Synthetic miRNAs may also be primary miRNAs having a 5′ cap sequence and poly-A tail. Synthetic miRNAs may be expressed from an expression vector or provided as oligonucleotides. The CAGRN sequence (the R-SBE sequence) may be located within a range of 4-12 by from the Drosha cleavage site. Also, the CAGRN sequence may be within a range of 4-12 bp, preferably about 9 bp, from the 5′ end of the mature miRNA. The miRNAs comprising a heterologous substantially double-stranded portion comprising the nucleotide sequence CAGRN that promotes binding of a receptor-specific SMAD (rSMAD) protein to the miRNA, wherein R is A or G and N is A, G, C, or U may be based (derived from) on any miRNA. In addition, sequences of synthetic miRNAs, e.g., seed sequences, may be based on (derived from) any miRNA. For example, the skilled artisan can identify miRNA seed sequences and cognate mRNA targets using a variety of algorithms known in the art. Exemplary algorithms are described in the following references: miRanda: Enright, A. J. et al. (2003) MicroRNA targets in Drosophila. Genome Biol. 5, R1; TargetScan: Lewis, B. P. et al. (2003) Prediction of mammalian microRNA targets. Cell 11, 787-798; TargetScanS: Lewis, B. P. et al. (2005) Conserved seed pairing, often flanked by adenosines, indicates that thousands of human genes are microRNA targets. Cell 120, 15-20; DIANA microT: Kiriakidou, M. et al. (2004) A combined computational-experimental approach predicts human microRNA targets. Genes Dev. 18, 1165-1178; PicTar: Krek, A. et al. (2005) Combinatorial microRNA target predictions. Nat. Genet. 3, 495-500; RNAHybrid: Rehmsmeier, M. et al. (2004) Fast and effective prediction of microRNA/target duplexes. RNA 10, 1507-1517; STarMir: Long, D. et al (2007) Potent effect of target structure on microRNA function. Nat. Struct. and Mol. Bio. 14, 287-294; RNA22: Huynh, T. et al. (2006) A pattern-based method for the identification of microRNA-target sites and their corresponding RNA/RNA complexes. Cell 126, 1203-1217.

Exemplary miRNAs from which sequences (e.g., seed sequences) of the foregoing miRNAs may be derived include: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-71, hsa-let-71*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-36′-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518P, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548l, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*.

As discussed above some of the TGF-β/BMP/miR inhibitors and/or activators are vectors including a nucleic acid encoding for an activator or inhibitor molecule (e.g., miRNAs, SMADs, SMAD inhibitors) operably joined to a expression regulatory sequence. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes or portions thereof.

An expression vector is one into which a desired sequence may be inserted, e.g., by restriction and ligation such that it is operably joined to regulatory sequences and may be expressed as an RNA transcript. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays known in the art (e.g., β-galactosidase or alkaline phosphatase), and genes that visibly affect the phenotype of transformed or transfected cells, hosts, colonies or plaques (e.g., green fluorescent protein).

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide. It will be appreciated that a coding sequence need not encode a protein but may instead, for example, encode an oligonucleotide such as an exogenous miRNA.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art. One of skill in the art will be aware of appropriate regulatory sequences for expression of peptides and interfering RNA, e.g., shRNA, miRNA, etc.

The vectors of the invention may include nucleic acids encoding an shRNA, shRNA-mir, or microRNA molecules in a genomically integrated transgene or a plasmid-based expression vector. Thus, in some embodiments a molecule capable of inhibiting mRNA expression, preferably TGF miRNA expression, or microRNA activity, is a transgene or plasmid-based expression vector that encodes an oligonucleotide. Such transgenes and expression vectors can employ either polymerase II or polymerase III promoters to drive expression of these oligonucleotides and result in functional expression (e.g., exogenous miRNA expression) in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

In some embodiments, a virus vector for delivering a nucleic acid molecule is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), lentiviral vectors (Naldini L, et al., Proc Natl Acad Sci USA. 1996 Oct. 15; 93(21):11382-8) and Ty virus-like particle (Allsopp et al., Eur. J. Immunol 26:1951-1959, 1996).

Another virus useful for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transduction. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other useful viral vectors are based on non-cytopathic eukaryotic viruses in which non-essential genes have been replaced with the gene of interest. Non-cytopathic viruses include certain retroviruses, the life cycle of which involves reverse transcription of genomic viral RNA into DNA with subsequent proviral integration into host cellular DNA. In general, the retroviruses are replication-deficient (i.e., capable of directing synthesis of the desired transcripts, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Clifton, N.J. (1991).

Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. Other examples include: N-TERT™ Nanoparticle Transfection System by Sigma-Aldrich, FectoFly™ transfection reagents for insect cells by Polyplus Transfection, Polyethylenimine “Max” by Polysciences, Inc., Unique, Non-Viral Transfection Tool by Cosmo Bio Co., Ltd., Lipofectamine™ LTX Transfection Reagent by Invitrogen, SatisFection™ Transfection Reagent by Stratagene, Lipofectamine™ Transfection Reagent by Invitrogen, FuGENE® HD Transfection Reagent by Roche Applied Science, GMP compliant in vivo-jetPEI™ transfection reagent by Polyplus Transfection, and Insect GeneJuice® Transfection Reagent by Novagen.

The TGF-β/BMP signaling pathway is a potent regulator of the cell cycle in many cell types. Aberrant TGF-β/BMP activity can cause numerous disease states. For instance when an interruption occurs in the TGF-β/BMP pathway resulting in less signaling a disease state can occur. Additionally other physiological conditions may not have involved an interruption in the TGF-β/BMP pathway but may benefit from additional pathway stimulation. Such disorders in which an activation of the TGF-β/BMP is desirable are referred to herein as TGF-β/BMP sensitive disorders. These disorders can be treated using a TGF-β/BMP pathway activator of the invention. TGF-β/BMP sensitive disorders include but are not limited to smooth muscle cell disorders, injuries associated with wounds, and metabolic bone disorders.

The TGF-β/BMP signaling pathway is a potent regulator of vascular smooth muscle (VSM) and endothelial cells and, as a result, TGF-β/BMP signaling is believed to play an important role in smooth muscle disorders such as vascular proliferative process i.e. angiogenesis. A smooth muscle disorder as used herein refers to a pathological condition in which the TGF-β/BMP signaling pathway in smooth muscle cells is reduced compared to normal smooth muscle cells. Smooth muscle disorders include but are not limited to restenosis, atherosclerosis, coronary heart disease, thrombosis, myocardial infarction, stroke, smooth muscle neoplasms such as leiomyoma and leiomyosarcoma of the bowel and uterus, uterine fibroid or fibroma, obliterative disease of vascular grafts and transplanted organs, arterial hypertension, hereditary haemorrhagic telangiectasia, unstable angina, chronic stable angina, transient ischemic attacks, peripheral vascular disease, preeclampsia, deep venous thrombosis, embolism, disseminated intravascular coagulation or thrombotic cytopenic purpura. Smooth muscle disorders also include vascular injury, an injury arising by any means including, but not limited to, procedures such as angioplasty, carotid endarterectomy, post CABG (coronary artery bypass graft) surgery, vascular graft surgery, stent placements or insertion of endovascular devices and prostheses.

The TGF-β/BMP signaling pathway is also a potent regulator of bone tissue and, as a result, TGF-β/BMP signaling is believed to play an important role in metabolic bone disorders. A metabolic bone disorder as used herein refers to a pathological condition in which the TGF-β/BMP signaling pathway in bone tissue is reduced compared to normal bone tissue. Metabolic bone disorders include but are not limited to osteopenia, osteoporosis, Paget's Disease (osteitis deformans), osteomalacia, rickets, tumor-associated bone loss, hypophosphatasia, drug-induced osteomalacia, and renal osteodystrophy.

TGF-β/BMP pathway activators may also be used to promote wound healing. Wounds are generally defects in the protective covering of an individual organ or organ system. Without this physiological barrier, the tissue normally protected by the covering is subject to loss of biologic compartmentalization. When tissue is no longer physiologically compartmentalized it is subject to fluid loss, invasion by microorganisms, electrolyte imbalances, and in some cases metabolic dysfunction. The term “wound,” for purposes herein, refers broadly to an injury to an organ or organ system. In the case of the skin, the injury may be to the epidermis, the dermis and/or the subcutaneous tissue. Skin wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum). The term “partial thickness wound” refers to wounds that encompass Grades I-III; examples of partial thickness wounds include burn wounds, pressure sores, venous stasis ulcers, and diabetic ulcers. The term “deep wound” includes both Grade III and Grade IV wounds. The methods of the invention are useful for treating all grades of wounds, including chronic and acute wounds. The term “chronic wound” refers to a wound that has not healed within 30 days.

The term “promoting wound healing,” for purposes herein, refers to enabling reconstitution of the normal physiologic barrier of an organ or organ system. In the case of skin wounds, promoting would healing may include the induction of the formation of granulation tissue, and/or the induction of wound contraction, and/or the induction of revascularization, and/or the induction of epithelialization (i.e., the generation of new cells in the epithelium).

The types of wounds to be treated by the methods of the invention include various kinds of wounds including, but are not limited to: surgical wounds; traumatic wounds; radiation injury wounds; toxic epidermal necrolysis wounds; infectious wounds; neoplastic wounds; full-thickness wounds; partial-thickness wounds; and burn wounds, as well as wounds arising from various types of ulcers, such as skin ulcers, corneal ulcers, arterial obstructive ulcers, continuous pressure-induced decubital and diabetic ulcers, burn ulcers, injury ulcers, radiation ulcers, drug-induced ulcers, post-operative ulcers, inflammatory ulcers, ulcers of the gastrointestinal tract, simple ulcers and other types of angiopathic ulcers, and chronic (intractable) ulcers.

Aberrant over-activity of the TGF-β/BMP pathway is also associated with numerous disease states. Such disorders in which inhibition of the TGF-β/BMP pathway is desirable are referred to herein as TGF-β/BMP mediated disorders. These disorders can be treated using a TGF-β/BMP pathway inhibitor of the invention. TGF-β/BMP mediated disorders include but are not limited to fibroproliferative diseases, cancer, neurological conditions and excessive scar formation.

The TGF-β/BMP signaling pathway is involved in fibroblast regulation and over activity of the pathway is associated with aberrant fibroblast mediated conditions. As a result, TGF-β/BMP signaling is believed to play an important role in fibroproliferative disorders. A fibroproliferative disorder as used herein refers to a pathological condition in which the TGF-β/BMP signaling pathway in fibroblasts is reduced compared to normal fibroblasts. Fibroproliferative disorders include but are not limited to kidney disorders associated with unregulated TGF-β activity and excessive fibrosis, including glomerulonephritis (GN), such as mesangial proliferative GN, immune GN, and crescentic GN. Other renal conditions that can be treated by inhibitors of TGF-β intracellular signaling pathway include diabetic nephropathy, renal interstitial fibrosis, renal fibrosis in transplant patients receiving cyclosporine, and HIV-associated nephropathy. Lung fibroses resulting from excessive TGF-β activity include adult respiratory distress syndrome, idiopathic pulmonary fibrosis, and interstitial pulmonary fibrosis often associated with autoimmune disorders, such as systemic lupus erythematosus and scleroderna, chemical contact, or allergies. Another autoimmune disorder associated with fibroproliferative characteristics is rheumatoid arthritis. Eye diseases associated with a fibroproliferative condition include retinal reattachment surgery accompanying proliferative vitreoretinopathy, cataract extraction with intraocular lens implantation, and post glaucoma drainage surgery.

The modulation of immune and inflammation systems by TGF-β includes stimulation of leukocyte recruitment, cytokine production, and lymphocyte effector function, and inhibition of T-cell subset proliferation, 3-cell proliferation, antibody formation, and monocytic respiratory burst. Wahl et al., Immunol Today, 1989, 10, 258-61. TGF-β plays an important role in the pathogenesis of lung fibrosis which is a major cause of suffering and death seen in pulmonary medicine based on its strong extracellular matrix inducing effect. The association of TGF-β with human lung fibrotic disorders has been demonstrated in idiopathic pulmonary fibrosis, autoimmune lung diseases and bleomycin induced lung fibrosis. Nakao et al., J. Clin. Inv., 1999, 104, 5-11.

Neurological conditions characterized by TGF-β/BMP production include CNS injury after traumatic and hypoxic insults, Alzheimer's disease, and Parkinson's disease.

Other conditions that are potential clinical targets for TGF-β/BMP inhibitors include myelofibrosis, tissue thickening resulting from radiation treatment, nasal polyposis, polyp surgery, liver cirrhosis, and osteoporosis.

Cancers are also TGF-β/BMP mediated disorders. “Cancer” as used herein refers to an uncontrolled growth of cells which interferes with the normal functioning of the bodily organs and systems. Cancers which migrate from their original location and seed vital organs (e.g., metastatic cancer) can eventually lead to the death of the subject through the functional deterioration of the affected organs. Carcinomas are malignant cancers that arise from epithelial cells and include adenocarcinoma and squamous cell carcinoma. Sarcomas are cancer of the connective or supportive tissue and include osteosarcoma, chondrosarcoma and gastrointestinal stromal tumor. Hematopoietic cancers, such as leukemia, are able to outcompete the normal hematopoietic compartments in a subject, thereby leading to hematopoietic failure (in the form of anemia, thrombocytopenia and neutropenia) ultimately causing death. A person of ordinary skill in the art can classify a cancer as a sarcoma, carcinoma or hematopoietic cancer.

Cancer, as used herein, includes the following types of cancer, breast cancer, biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chromic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art.

In some aspects, the invention provides compositions and methods for inhibiting maturation of primary miRNAs (e.g., at least one miRNA) in a cell which is in vivo or in vitro. As used herein, cells include, but are not limited to: vascular smooth muscle cells, fat cells, bone cells, cartilage cells, skin cells, pancreatic cells, gastric cells, germ cells, hepatic cells, red blood cells, white blood cells, cardiac muscle cells, skeletal muscle cells, osteoblasts, skeletal myoblasts, neuronal cells, vascular endothelial cells, pigment cells, fibroblasts and the like. In some cases, the cell may be a stem cell that has the ability to proliferate in culture, producing daughter cells that remain relatively undifferentiated, and other daughter cells that differentiate giving rise to cells of one or more specialized cell types.

In some embodiments, the cells are mammalian cells, e.g., human cells or non-human animal cells, e.g., cells of non-human primate, rodent (e.g., mouse, rat, guinea pig, rabbit), origin, or interspecies hybrids. In certain embodiments the cells are obtained from a biopsy (e.g., tissue biopsy, fine needle biopsy, etc.) or at surgery for TGFβ/BMP mediated disorder.

In some embodiments, cells of the invention may be derived from a cancer. In some embodiments the cancer is a cancer associated with a known or characteristic genetic mutation or polymorphism such as a deletion in the SMAD4 gene. In some cases, the cells are cancer stem cells that lack the normal growth regulatory mechanisms that limit the uncontrolled proliferation of stem cells. Cancer stem cells are capable of proliferation, are clonogenic, and in some cases are identifiable by certain biomarkers. Exemplary cancer stem cell biomarkers include CD20, CD24, CD34, CD38, CD44, CD45, CD105, CD133, CD166, EpCAM, ESA, SCA1, Nestin, Pecam, and Stro1.

Cells can be primary cells, non-immortalized cell lines, immortalized cell lines, transformed immortalized cell lines, benign tumor derived cells or cell lines, malignant tumor derived cells or cell lines, transgenic cell lines, etc. In some embodiments the tumor is a metastatic tumor, in which case the cells may be derived from the primary tumor or a metastasis. In some embodiments, cells of the invention are present in or derived from noncancerous tissue. Such tissues include, for example, tissues found in the breast, gastrointestinal tract (stomach, small intestine, colon), liver, biliary tract, bronchi, lungs, pancreas, kidneys, ovaries, prostate, skin, cervix, uterus, bladder, ureter, testes, exocrine glands, endocrine glands, blood vessels, etc.

One aspect of the invention contemplates the treatment of a individual having or at risk of having a TGF-β/BMP mediated disorder or a TGF-β/BMP sensitive disorder. As used herein an individual, also referred to as a subject, is a mammalian species, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. Preferred subjects are human subjects (individuals). The human subject may be a pediatric, adult or a geriatric subject.

As used herein, the term “treating” and “treatment” refers to modulating certain areas of the body so that the subject has an improvement in the disease, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease.

The agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a patient, such as a syringe, topical application devices, or iv needle tubing and bag.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The TGF/BMP modulators (activators or inhibitors) can be combined with other therapeutic agents such as anti-cancer agents, drugs for the treatment of fibroproliferative disease, drugs for the treatment of smooth muscle disorders. and wound healing agents. Thus, compositions of the combinations are envisioned according to the invention.

The modulators and other therapeutic agent may be administered simultaneously or sequentially. When the other therapeutic agents are administered simultaneously they can be administered in the same or separate formulations, but are administered at the same time. The other therapeutic agents are administered sequentially with one another and with the modulators, when the administration of the other therapeutic agents and the modulators is temporally separated. The separation in time between the administration of these compounds may be a matter of minutes or it may be longer.

The pharmaceutical compositions of the present invention preferably contain a pharmaceutically acceptable carrier or excipient suitable for rendering the compound or mixture administrable orally as a tablet, capsule or pill, or parenterally, intravenously, intradermally, intramuscularly or subcutaneously, or transdermally. The active ingredients may be admixed or compounded with any conventional, pharmaceutically acceptable carrier or excipient. The compositions may be sterile.

As used herein, the term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations. A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18th Ed. (1990).

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

An effective amount, also referred to as a therapeutically effective amount, of an TGF-β/BMP/miR modulator (for example, an oligonucleotide molecule capable of inhibiting or supplementing expression of miRNA associated with a TGF-β/BMP Mediated disorder) is an amount sufficient to ameliorate at least one adverse effect associated with expression, or reduced expression, of the microRNA in a cell or in an individual in need of such inhibition or supplementation. The therapeutically effective amount to be included in pharmaceutical compositions depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic agent being administered, the size of the subject, or the severity of the disease or condition. One of ordinary skill in the art can empirically determine the effective amount of a particular nucleic acid and/or other therapeutic agent without necessitating undue experimentation.

The pharmaceutical compositions containing oligonucleotides and/or other compounds can be administered by any suitable route for administering medications. A variety of administration routes are available. The particular mode selected will depend, of course, upon the particular agent or agents selected, the particular condition being treated, and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed herein. For use in therapy, an effective amount of the nucleic acid and/or other therapeutic agent can be administered to a subject by any mode that delivers the agent to the desired surface, e.g., mucosal, systemic.

Administering the pharmaceutical composition of the present invention may be accomplished by any means known to the skilled artisan. Routes of administration include but are not limited to oral, parenteral, intravenous, intramuscular, intraperitoneal, intranasal, sublingual, intratracheal, inhalation, subcutaneous, ocular, vaginal, and rectal. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulizers.

In some cases, compounds of the invention are prepared in a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vesicles (LUVs), which range in size from 0.2-4.0 μm can encapsulate large macromolecules. RNA, DNA and intact virions can be encapsulated within the aqueous interior and be delivered to cells in a biologically active form. Fraley et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to, for example, an smooth muscle cell include, but are not limited to: intact or fragments of molecules which interact with smooth muscle cell specific receptors and molecules, such as antibodies, which interact with the cell surface markers of cancer cells. Such ligands may easily be identified by binding assays well known to those of skill in the art. In still other embodiments, the liposome may be targeted to a tissue by coupling it to an antibody known in the art.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

Certain cationic lipids, including in particular N-[1-(2,3 dioleoyloxy)-propyl]-N,N,N-trimethylammonium methyl-sulfate (DOTAP), appear to be especially advantageous when combined with the modified oligonucleotide analogs of the invention.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”. PCT/US/0307 describes a biocompatible, preferably biodegradable polymeric matrix for containing an exogenous gene under the control of an appropriate promoter. The polymeric matrix can be used to achieve sustained release of the therapeutic agent in the subject.

The polymeric matrix preferably is in the form of a microparticle such as a microsphere (wherein the nucleic acid and/or the other therapeutic agent is dispersed throughout a solid polymeric matrix) or a microcapsule (wherein the nucleic acid and/or the other therapeutic agent is stored in the core of a polymeric shell). Other forms of the polymeric matrix for containing the therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the nucleic acid are administered to the subject via an implant while the other therapeutic agent is administered acutely. Biocompatible microspheres that are suitable for delivery, such as oral or mucosal delivery, are disclosed in Chickering et al. (1996) Biotech Bioeng 52:96-101 and Mathiowitz E et al. (1997) Nature 386:410-414 and PCT Pat. Application WO97/03702.

Both non-biodegradable and biodegradable polymeric matrices can be used to deliver the nucleic acid and/or the other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the nucleic acid agents. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

If the therapeutic agent is a nucleic acid, the use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g., in saline or buffer) or using any delivery vehicle known in the art. For instance the following delivery vehicles have been described: cochleates; Emulsomes; ISCOMs; liposomes; live bacterial vectors (e.g., Salmonella, Escherichia coli, Bacillus Calmette-Guérin, Shigella, Lactobacillus); live viral vectors (e.g., Vaccinia, adenovirus, Herpes Simplex); microspheres; nucleic acid vaccines; polymers (e.g. carboxymethylcellulose, chitosan); polymer rings; proteosomes; sodium fluoride; transgenic plants; virosomes; and, virus-like particles.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in to dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R (1990) Science 249:1527-1533, which is incorporated herein by reference.

The compounds may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In some embodiments, tissue engineering is performed using a scaffold material that allows for attachment of cells. The scaffold material contains a molecule (e.g., conjugated to scaffold) such as a TGFβ/BMP/miR modulator (e.g, TGFβ/BMP/miR activator or inhibitor) that promotes the production of extracellular matrix proteins and/or proliferation. In the preferred embodiment, the scaffold is formed of synthetic or natural polymers, although other materials such as hydroxyapatite, silicone, and other inorganic materials can be used. The scaffold may be biodegradable or non-degradable. Representative synthetic non-biodegradable polymers include ethylene vinyl acetate and poly(meth)acrylate. Representative biodegradable polymers include polyhydroxyacids such as polylactic acid and polyglycolic acid, polyanhydrides, polyorthoesters, and copolymers thereof. Natural polymers include collagen, hyaluronic acid, and albumin. Hydrogels are also suitable. A particularly useful hydrogel forming material is a polyethylene glycol-diacrylate polymer, which is photopolymerized. Other hydrogel materials include calcium alginate and certain other polymers that can form ionic hydrogels that are malleable and can be used to encapsulate cells. Exemplary tissue engineering methods are well known in the art, such as those disclosed in WO/2002/016557, USPatent App. 20050158358, and U.S. Pat. No. 6,103,255 the contents of which are incorporated herein in their entirety.

The scaffolds are used to produce new tissue, such as vascular tissue, bone, cartilage, tendons, and ligaments. The scaffold is typically seeded with the cells; the cells are cultured; and then the scaffold implanted. Alternatively, as noted above, the scaffold is sprayed into or onto a site such as a joint lining, seeded with cells, and then the site is closed surgically. Liquid polymer-cell suspensions can also be injected into a site, such as within a joint, where the material may be polymerized. Applications include the repair and/or replacement of organs or tissues, such as blood vessels, cartilage, joint linings, tendons, or ligaments, or the creation of tissue for use as “bulking agents”, which are typically used to block openings or lumens, or to shift adjacent tissue, as in treatment of reflux.

Methods for detecting aberrant TGF/BMP signaling in a subject are also provided herein. The methods typically comprise obtaining a biological sample of the subject, determining levels in the sample of a plurality of TGF miRNAs, and if levels of at least a subset of the TGF miRNAs are above control levels, detecting aberrant TGF/BMP signaling in the subject. The detection of aberrant TGF/BMP signaling may be predictive of the subject having a TGF-β/BMP mediated disorder, such as a fibroproliferative disorder, a cancer, or an autoimmune disease. The TGF miRNAs may be selected from the group consisting of: hsa-miR-21, hsa-miR-148a, hsa-miR-18a, hsa-miR-127-5p, hsa-miR-23a, hsa-miR-105, hsa-miR-148b, hsa-miR-106b, hsa-miR-134, hsa-miR-23b, hsa-miR-199a-5p, hsa-miR-152, hsa-miR-410, hsa-miR-103, hsa-miR-195, hsa-miR-542-3p, hsa-miR-107, hsa-miR-215, hsa-miR-339-3p, hsa-miR-140-3p, hsa-miR-342-3p, hsa-miR-423-5p, hsa-miR-421, hsa-miR-361-5p, hsa-miR-452, hsa-miR-509-5p, hsa-miR-331-5p, hsa-miR-345, hsa-miR-600, hsa-miR-422a, hsa-miR-518e, hsa-miR-487a, hsa-miR-631, hsa-miR-487b, and hsa-miR-654-5p.

Any appropriate control level may be used for detecting aberrant TGF/13 MP signaling. For example, a control level of a TGF miRNA may be the level of the TGF miRNA in a tissue (e.g., a non-cancerous tissue, a non-metastatic cancer, a healthy tissue) that does not have aberrant TGF/BMP signaling. Biological samples used in the methods described herein (e.g., diagnostic, prognostic, therapeutic, etc., or any combination thereof) may comprise cells from the eye, ear, nose, teeth, tongue, epidermis, epithelium, blood, tears, saliva, mucus, urinary tract, urine, muscle, cartilage, skin, or any other tissue or bodily fluid from which sufficient DNA, RNA, protein, or other molecule or combinations of molecules can be obtained. In some embodiments, the biological sample is a cancer tissue. MiRNAs levels in a sample may be detected using any one of a number of methods known in the art, including, but not limited to RT-PCR, northern blot analysis, array analysis, or bead-based miRNA detection. Other appropriate methods will be known to the skilled artisan. Typically the methods, e.g., array analysis and bead-based analysis, allow for parallel detection of multiple miRNAs.

Accordingly, oligonucleotide arrays are provided herein for determining levels of multiple miRNAs in parallel. In some embodiments, the oligonucleotide arrays comprise (or consist essentially of) immobilized probes that hybridize with TGF miRNAs, and optionally one or more control probes. The TGF miRNAs which may be detected by the oligonucleotide arrays may be selected from the group consisting of: hsa-miR-21, hsa-miR-148a, hsa-miR-18a, hsa-miR-127-5p, hsa-miR-23a, hsa-miR-630, hsa-miR-105, hsa-miR-148b, hsa-miR-106b, hsa-miR-134, hsa-miR-23b, hsa-miR-648, hsa-miR-199a-5p, hsa-miR-152, hsa-miR-410, hsa-miR-198, hsa-miR-103, hsa-miR-659, hsa-miR-214, hsa-miR-195, hsa-miR-542-3p, hsa-miR-330-3p, hsa-miR-107, hsa-miR-671-3p, hsa-miR-215, hsa-miR-298, hsa-miR-607, hsa-miR-339-3p, hsa-miR-140-3p, hsa-miR-770-5p, hsa-miR-300, hsa-miR-342-3p, hsa-miR-1298, hsa-miR-423-5p, hsa-miR-188-3p, hsa-miR-877, hsa-miR-421, hsa-miR-361-5p, hsa-miR-1539, hsa-miR-452, hsa-miR-220c, hsa-miR-933, hsa-miR-509-5p, hsa-miR-378, hsa-miR-508-5p, hsa-miR-331-5p, hsa-miR-940, hsa-miR-509-3-5p, hsa-miR-383, hsa-miR-516a-3p, hsa-miR-345, hsa-miR-1205, hsa-miR-600, hsa-miR-422a, hsa-miR-518e, hsa-miR-487a, hsa-miR-1207-5p, hsa-miR-631, hsa-miR-541, hsa-miR-520a-5p, hsa-miR-487b, hsa-miR-1266, hsa-miR-1208, hsa-miR-567, hsa-miR-525-5p, hsa-miR-498, hsa-miR-1290, hsa-miR-1284, hsa-miR-654-5p, hsa-miR-922, hsa-miR-513a-5p, hsa-miR-1321, hsa-miR-1292, hsa-miR-921, hsa-miR-1912, hsa-miR-612, hsa-miR-1909, hsa-miR-1324, hsa-miR-1324, hsa-miR-623, and hsa-miR-1915. Diagnostic kits comprising the oligonucleotide arrays are also provided.

### EXAMPLES

### Example 1

### MiR-21 is Critical for Modulation of VSMC Phenotype by BMP and TGFβ

Mutations in molecules of the TGFβ or BMP signaling pathways are found among patients with vascular disorders, indicating the essential role of TGFβ or BMP pathways in vascular homeostasis [Dijke, P. & Arthur, H. M., Nature Rev. Mol. Cell Biol. 8, 857-868 (2007) and Morrell, N. W., Proc Am Thorac Soc 3, 680-686 (2006)]. Both TGFβs and BMPs are known critical modulators of the VSMC phenotype [Owens, G. K., Physiol Rev 75, 487-517 (1995), Rensen, S. S. M., et al. Netherlands Heart 115, 100-108 (2007), and Lagna, G. et al., J Biol Chem 282, 37244-37255 (2007)]. Inhibition of TGFβ or BMP signaling in VSMCs decreases the expression of VSMC-specific genes and transforms VSMCs from a fully differentiated or “contractile” phenotype to a dedifferentiated or “synthetic” state [Rensen, S. S. M., et al. Netherlands Heart J. 15, 100-108 (2007), Lagna, G. et al., J Biol Chem 282, 37244-37255 (2007), and Owens, G. K., Kumar, M. S. & Wamhoff, Physiol Rev 84, 767-801 (2004)].

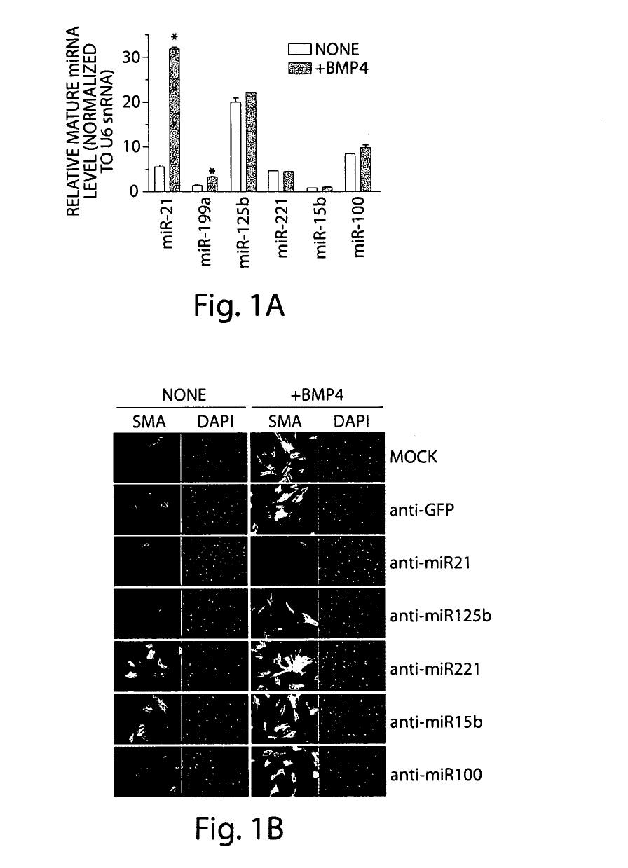

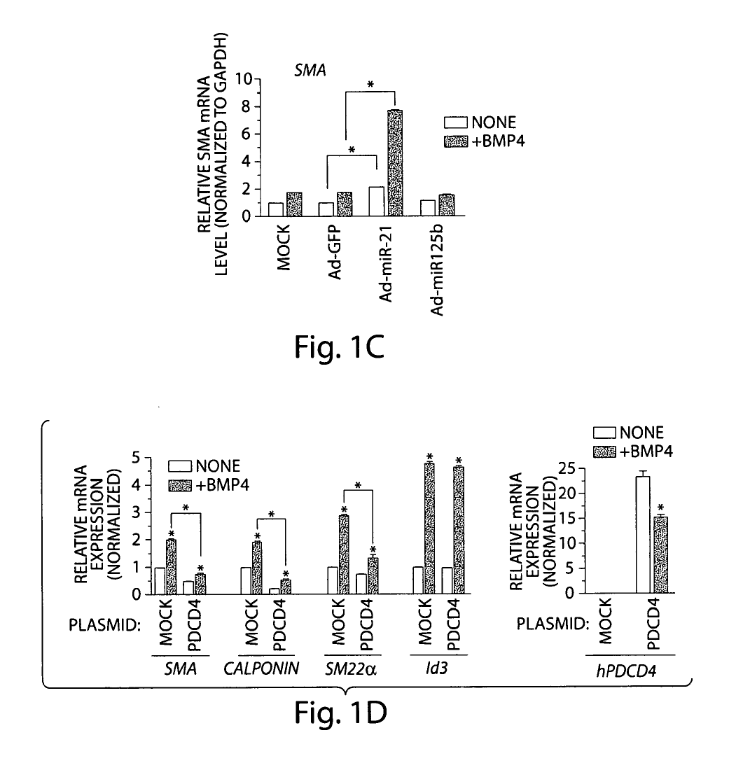

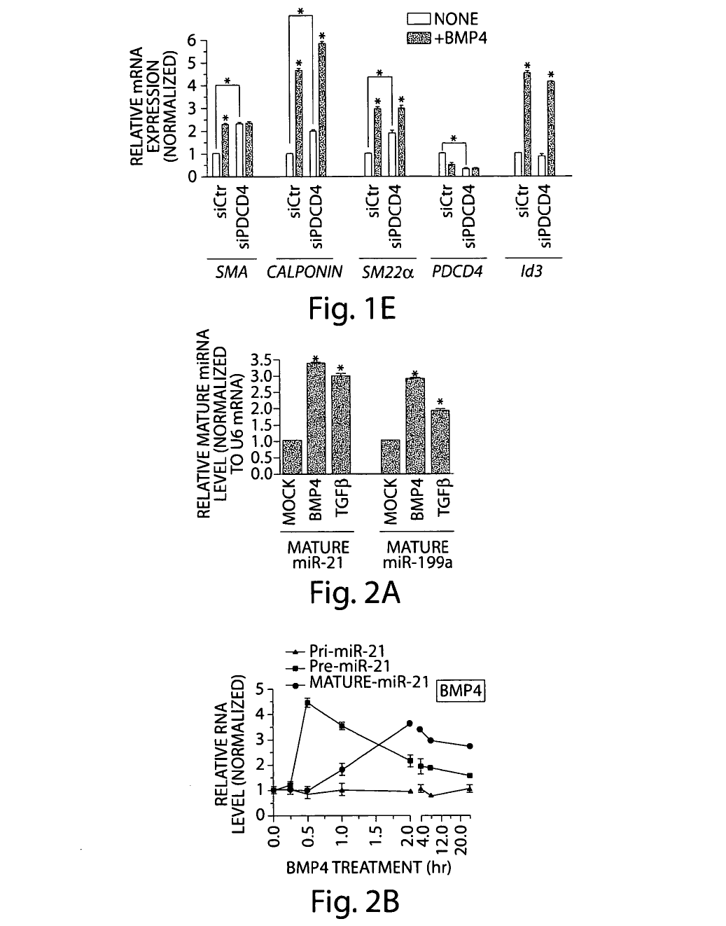

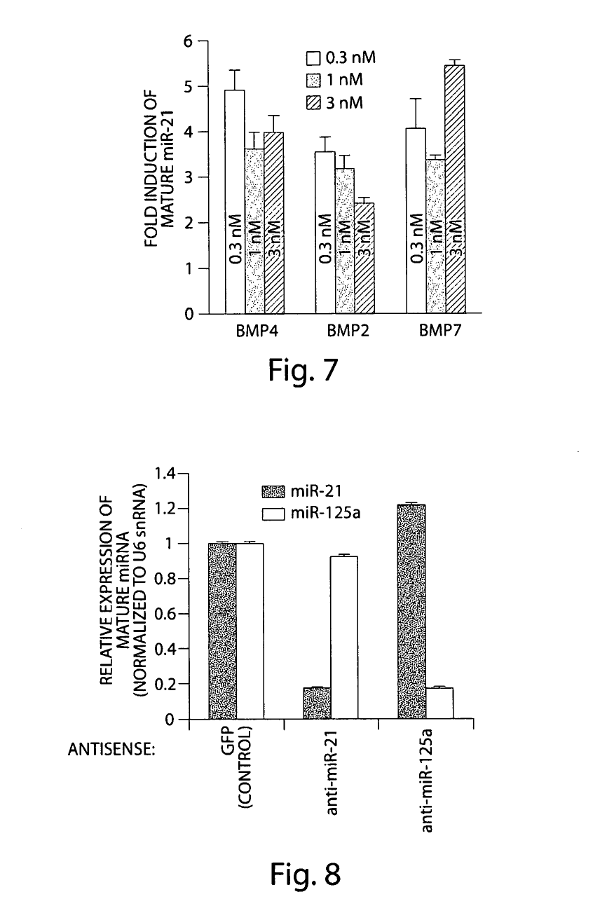

We investigated the involvement of miRNAs in the TGFβ family-mediated modulation of VSMC phenotype by cloning and comparing the relative abundance of miRNAs expressed in vehicle- and BMP4-treated human primary pulmonary artery smooth muscle cells (PASMCs) (FIG. 6). The expression level of a selected group of miRNAs was then directly measured by qRT-PCR upon 24 h of BMP4 stimulation (FIG. 1a): mature miR-21 and miR-199a showed a significant increase of expression (5.7-fold and 2.1-fold, respectively) in the presence of BMP4. MiR-21 was comparably induced by three BMP ligands that stimulate VSMC differentiation (BMP2, BMP4, and BMP7) [Lagna, G. et al., J Biol Chem 282, 37244-37255 (2007)]. (FIG. 7). Thus, a subset of miRNAs is induced by BMP signaling in VSMC. High expression of miR-21 has also been observed in the vascular wall of balloon-injured rat carotid arteries, an in vivo model recapitulating smooth muscle phenotype switch [Ji, R. et al., Circ Res 100, 1579-1588 (2007)].

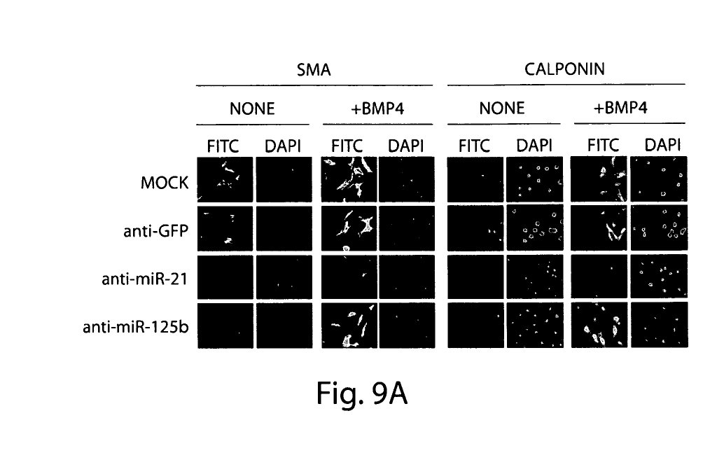

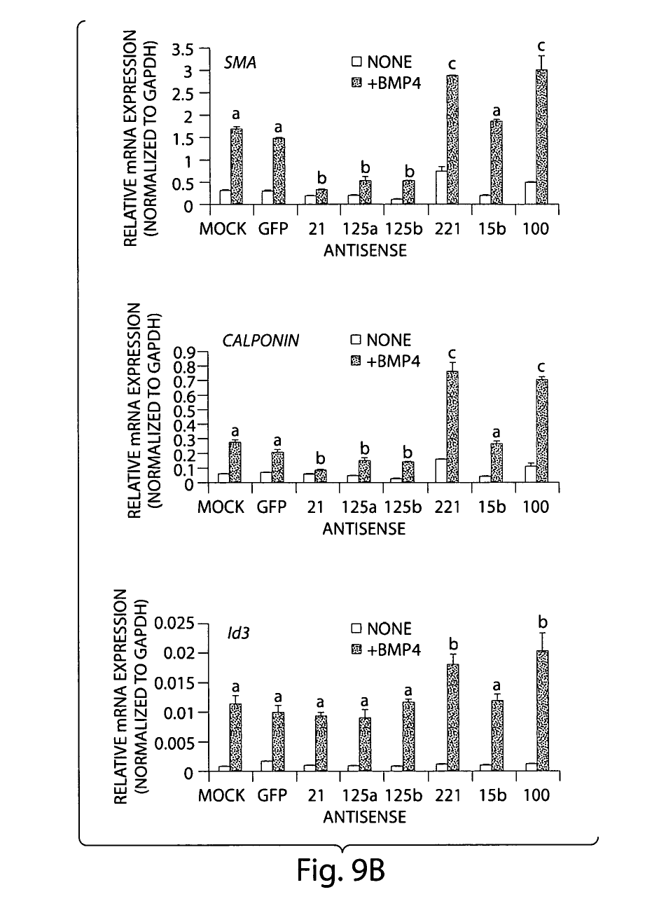

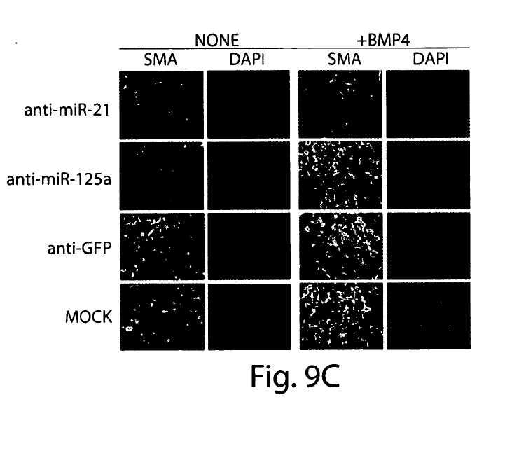

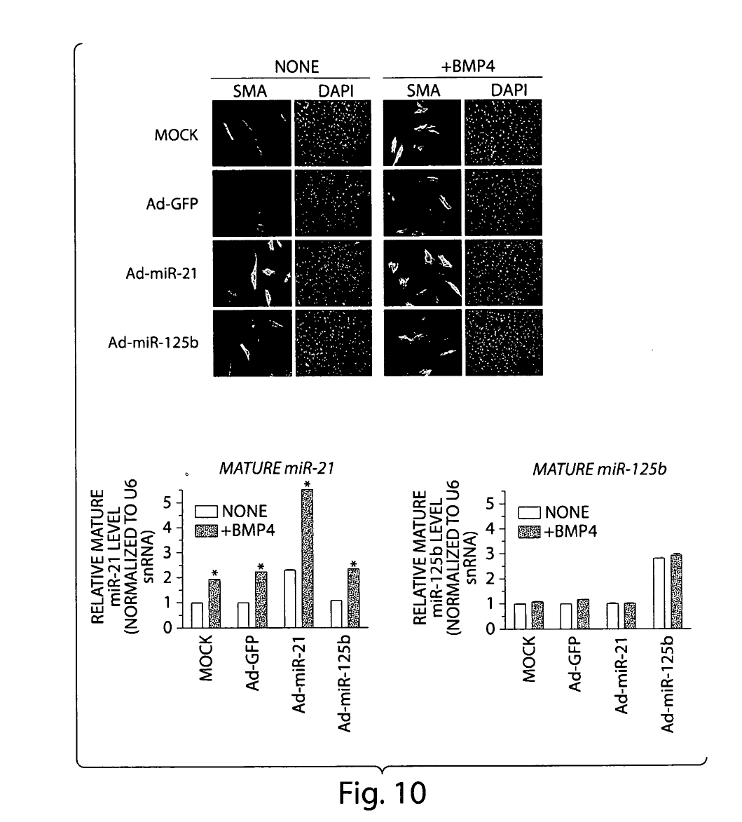

The function of miRNAs was tested by transfecting PASMCs with “anti-miRs”, 2′-O-methyl-modified RNA oligonucleotides complementary to individual miRNA sequences [Esau, C. C., Methods 44, 55-60 (2008).]. Anti-miR-21 specifically decreased mature miR-21 expression (FIG. 8) and effectively reduced both basal and BMP4-induced expression of the SMC markers smooth muscle β-actin (SMA) and calponin (FIG. 1b and FIGS. 9a,b), suggesting that miR-21 is necessary for SM-specific gene expression. Downregulation of different miRNAs showed specific effects: targeting miR-125a and miR-125b inhibited SMC markers (FIG. 1b and FIGS. 9a,b), while depletion of miR-221 and miR-15b stimulated basal SMA expression (FIG. 1b and FIG. 9b). Anti-miR-21 also decreased SMA in pluripotent mouse C3H10T1/2 (10T1/2) cells treated with BMP4 (FIG. 9c). In gain-of-function experiments, forced expression of miR-21 by infection with an adenoviral miR-21 construct (Ad-miR-21) [van Rooij, E. et al., Proc Natl Acad Sci USA 103, 18255-18260 (2006)] increased SMA protein and mRNA levels in PASMCs (FIG. 1c and FIG. 10). Thus, miR-21 is a critical mediator of SMC differentiation by BMP signaling.

### Example 2

### miR-21 Regulates VSMC Differentiation Through PDCD4

Because miR-21 has been shown to target the tumor suppressor gene PDCD4 and downregulate its expression in cancer cells [Asangani, I. A. et al., Oncogene in press (2008), Frankel, L. B. et al., J Biol Chem 283, 1026-1033 (2007), and Zhu, S. et al., Cell research 18, 350-359 (2008)], we asked whether PDCD4 mediates the effect of miR-21 in SMC. Forced expression of miR-21 and reduction of miR-21 by anti-miR-21 in PASMCs decreased and increased PDCD4 mRNA expression, respectively (FIGS. 11a,b), confirming that PDCD4 is a miR-21 target. BMP4 treatment reduced PDCD4 (˜30%) (FIGS. 11a,b) and anti-miR-21 abolished this effect (FIG. 11b), suggesting that PDCD4 is negatively regulated by BMP4 as a result of miR-21 induction. We next examined whether modulation of PDCD4 expression in PASMCs affects SMC marker expression. Transfection of a human PDCD4 expression construct, which includes a miR-21 target sequence in its 3′-untranslated region (UTR) [Frankel, L. B. et al., J Biol Chem 283, 1026-1033 (2007)]. (FIG. 11c), increased the expression of hPDCD4 in 10T1/2 cells (FIG. 1d, right panel) and inhibited basal and BMP4-induced expression of the SMC markers SMA, calponin, and SM22B, but not of Id3, a gene directly regulated by BMP4 [Hollnagel, A., et al. J Biol Chem 274, 19838-19845 (1999)], indicating that PDCD4 represses specifically SM genes, not BMP signaling in general (FIG. 1d, left panel). BMP4 treatment still significantly augmented SM gene expression and decreased ectopic PDCD4 mRNA, presumably through the 3′ UTR miR-21 target site (FIG. 1d). Conversely, PDCD4 knockdown (˜60%) by siRNA (siPDCD4) in PASMCs increased the basal expression of SMA, calponin, and SM22B approximately 2-fold (FIG. 1e). BMP4 failed to induce SMA over the basal level when PDCD4 was depleted in the cell (FIG. 1e), while the levels of calponin and SM22B were still induced by BMP4 treatment, suggesting that BMP4 induces calponin and SM22B in part through a PDCD4-independent mechanism [Lagna, G. et al., J Biol Chem 282, 37244-37255 (2007) and Chan, M. C. et al., Mol Cell Biol 27, 5776-5789 (2007)] (FIG. 1e). In conclusion, PDCD4 is a functional target of miR-21 involved in the BMP-mediated induction of SMC markers in VSMC.

### Example 3

### Post-Transcriptional Regulation of miR-21 by the BMP/TGFβ Pathway

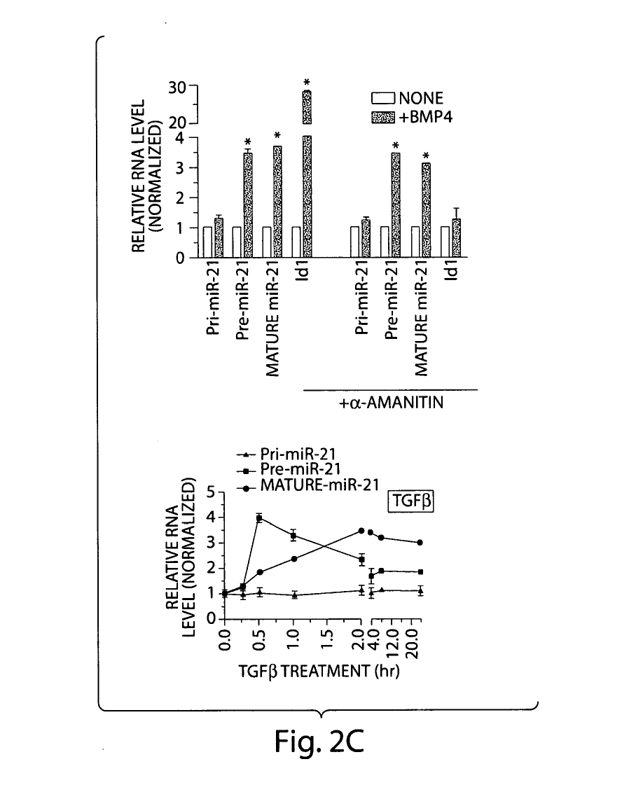

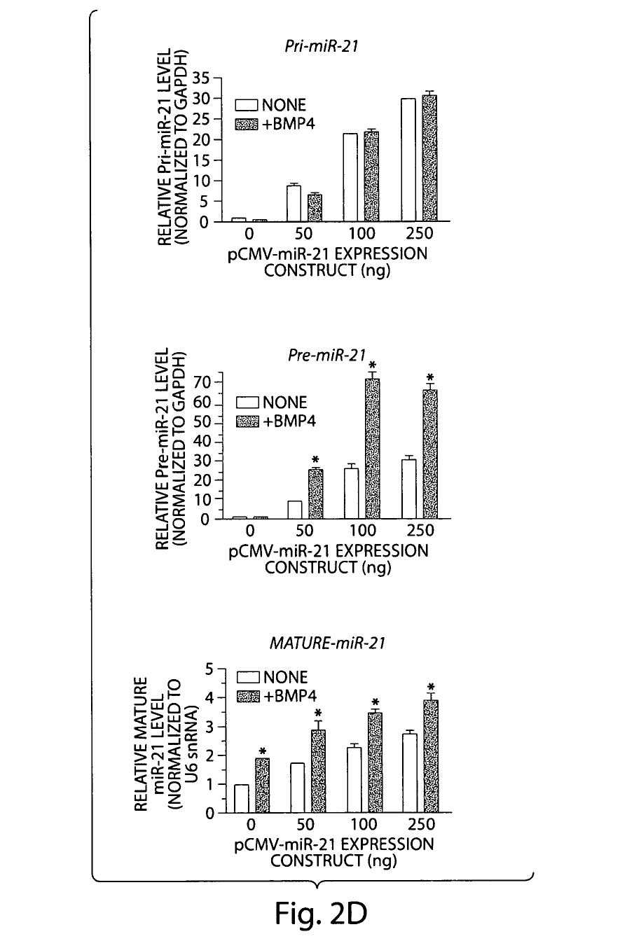

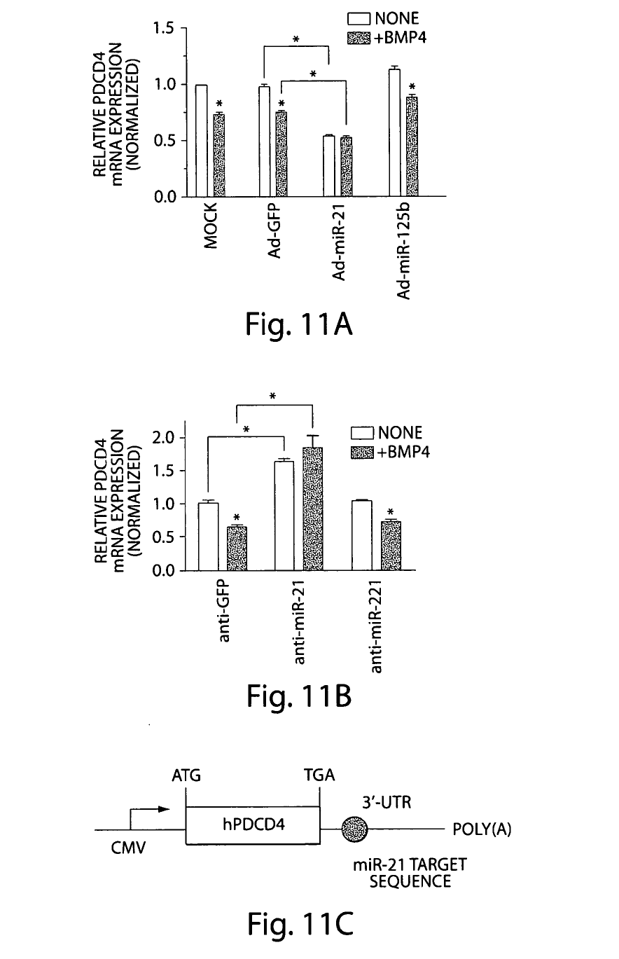

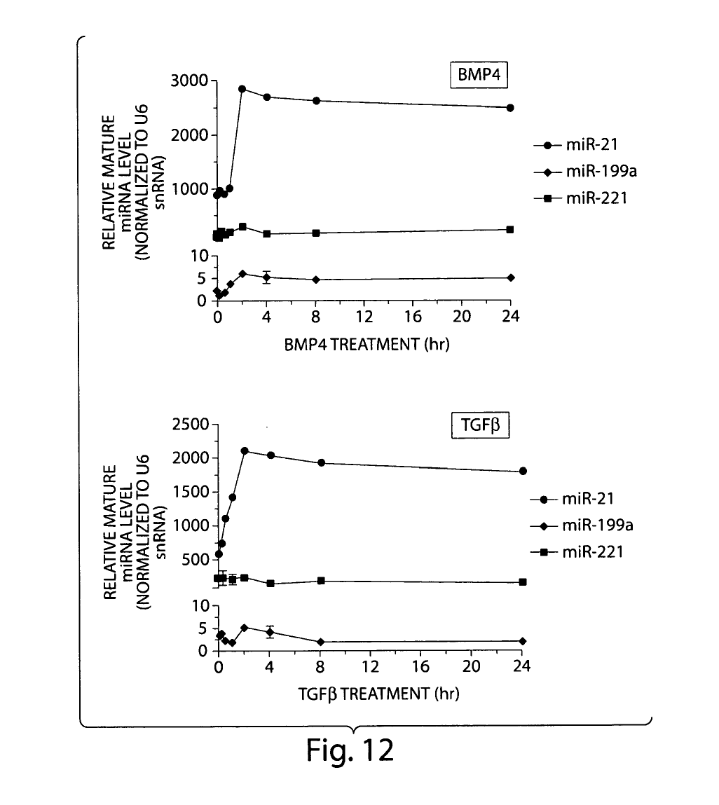

TGFβ, another inducer of the contractile phenotype [Owens, G. K., Physiol Rev 75, 487-517 (1995), Rensen, S. S. M., et al. Netherlands Heart J. 15, 100-108 (2007), and Lagna, G. et al., J Biol Chem 282, 37244-37255 (2007)], stimulated the expression of both miR-21 and miR-199a to a level comparable to BMP4 (FIG. 2a) with similarly fast kinetics (2 h) (FIG. 12), indicating that TGFβ and BMPs both support a contractile phenotype via elevation of miR-21.

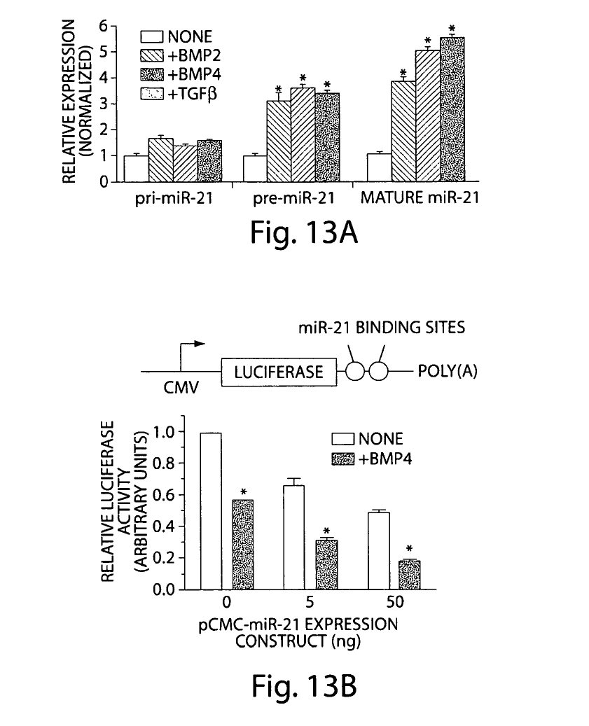

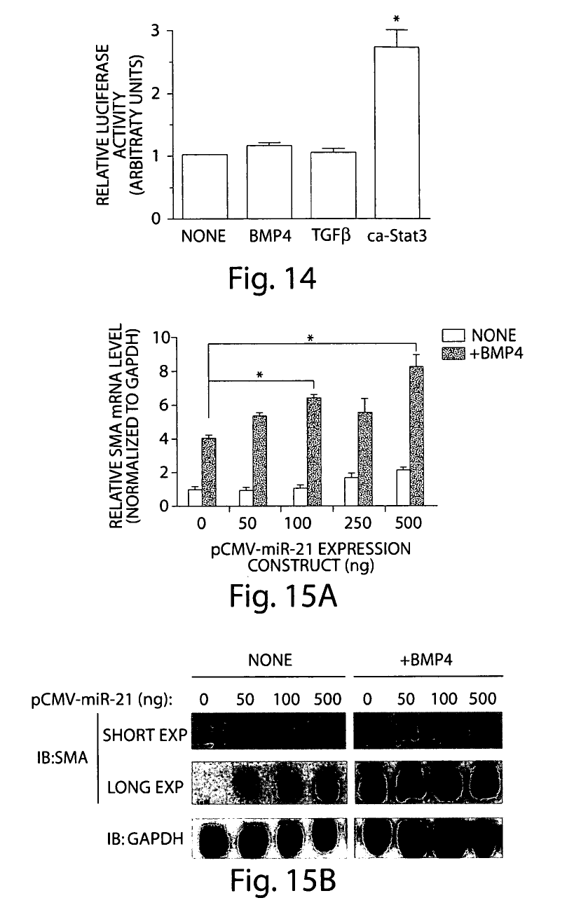

The biogenesis of miRNAs initiates with the transcription of the miRNA gene and proceeds with the cropping of the primary transcript (pri-miRNA) into a hairpin intermediate (pre-miRNA) by the nuclear ˜650 kDa microprocessor complex, comprised in humans of the RNase III Drosha [Lee, Y. et al., Nature 425, 415-419 (2003)], the DiGeorge syndrome critical region gene 8 (DGCR8) [Han, J. et al., Genes & development 18, 3016-3027 (2004) and Landthaler, M., et al. Curr Biol 14, 2162-2167 (2004)], and the DEAD box RNA helicases p68 and p72 (known also as DdxS and Ddx17) [Fukuda, T. et al., Nat Cell Biol 9, 604-611 (2007)]. The pre-miRNA is then exported from the nucleus and processed into a ˜22-nucleotide (nt) miRNA duplex by the cytoplasmic RNase III Dicer [Kim, V. N. & Nam, J. W., Trends Genet 22, 165-173 (2006), Kim, V. N., Nat Rev Mol Cell Biol 6, 376-385 (2005), and Zhao, Y. & Srivastava, D., Trends Biochem Sci 32, 189-197 (2007)]. Regulation of miRNA expression has been documented at the transcriptional level, but little is known about the stimuli and molecules regulating post-transcriptional processing [Kim, V. N. & Nam, J. W., Trends Genet 22, 165-173 (2006), Zhao, Y. & Srivastava, D., Trends Biochem Sci 32, 189-197 (2007), Lee, E. J. et al., RNA 14, 35-42 (2007), Obernosterer, G., et al. RNA 12, 1161-1167 (2006), Thomson, J. M. et al., Genes Dev. 20, 2202-2207 (2006), Wulczyn, F. G. et al., Faseb J 21, 415-426 (2007), and Guil, S. & Caceres, J. F., Nat Struct Mol Biol 14, 591-596 (2007)]. BMPs and TGFβs control gene expression through the Smad proteins, which embody the qualities of both signal transducers and transcriptional modulators [Massague, J., Seoane, J. & Wotton, D., Genes Dev. 19, 2783-2810 (2005) and Schmierer, B. & Hill, C. S., Nat Rev Mol Cell Biol 8, 970-982 (2007)], but are not known to affect RNA processing. Therefore, we examined the accumulation of primary miR-21 gene transcripts (pri-miR-21), pre-miR-21 and mature miR-21 upon BMP or TGFβ treatment in an expression time-course (FIG. 2b), expecting to find a transcriptional induction of pri-miR-21 transcripts in response to factor stimulation [Schmittgen, T. D. et al., Methods 44, 31-38 (2008)]. However, although we observed induction of mature miR-21 and pre-miR-21 2 h after BMP4, BMP2 or TGFβ treatment, we detected no significant change in the expression of pri-miR-21 after factor addition (FIG. 2b and FIG. 13a), suggesting that induction of miR-21 by BMP4, BMP2 or TGFβ occurs at a post-transcriptional step. Likewise, BMP4-mediated induction of both pre-miR-21 and mature miR-21 was resistant to inhibition of RNA polymerase II by β-amanitin, while induction of the BMP4 transcriptional target gene Id1 [Korchynskyi, O. & ten Dijke, P., J Biol Chem 277, 4883-4891 (2002)] was abolished (FIG. 2c). Furthermore, a luciferase reporter construct containing the miR-21 gene promoter was not activated by BMP4 or TGFβ treatment, while it was induced by its known regulator Stat3 (FIG. 14) [Loffler, D. et al., Blood 110, 1330-1333 (2007)].

A dose-dependent increase of all three forms of miR-21 was observed upon transfection in murine 10T1/2 cells of pCMV-miR-21, a plasmid in which human pri-miR-21 is transcribed from the cytomegalovirus (CMV) promoter [Zhu, S., et al. J Biol Chem 282, 14328-14336 (2007)] (FIG. 2d), indicating an expression level proportional to the episomal DNA copies. However, BMP4 could further induce pre-miR-21 and mature miR-21, but not pri-miR-21 (FIG. 2d), indicating that the miR-21 promoter or genomic locus is not required for post-transcriptional induction of miR-21 by BMP4. The plasmid-derived miR-21 induced by BMP4 was functional, because it repressed a miR-21 sensor construct containing complementary binding sites for the miR-21 sequence at the 3′-UTR of a luciferase reporter gene (FIG. 13b). Furthermore, expression of CMV-transcribed miR-21 induced SMA mRNA and protein in 10T1/2 cells in a dose-dependent manner, and was further increased by BMP4 stimulation (FIGS. 15a,b). Thus, the BMP4 pathway promotes the expression of precursor and functional mature miR-21 through a post-transcriptional, genome-independent mechanism.

### Example 4

### Ligand-Dependent Interaction of Smads with the RNA Helicase p68

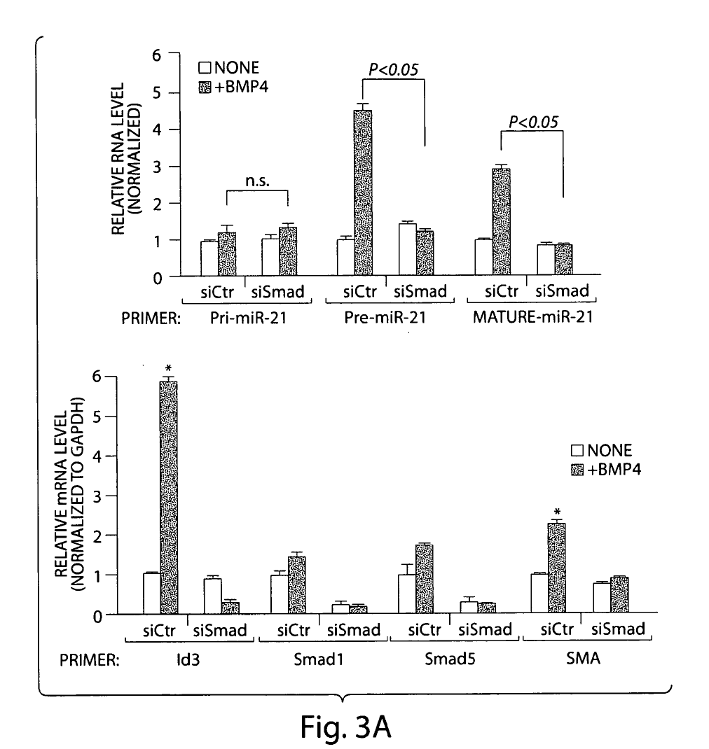

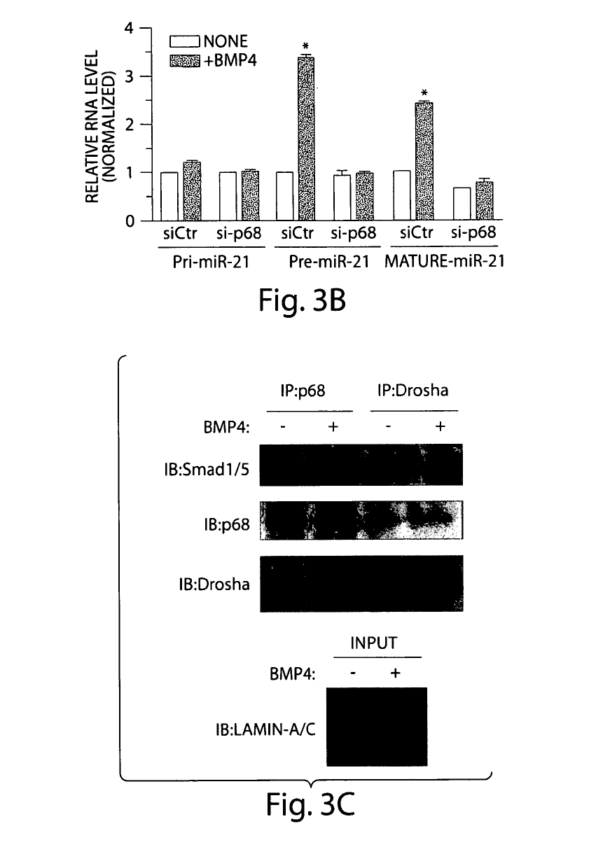

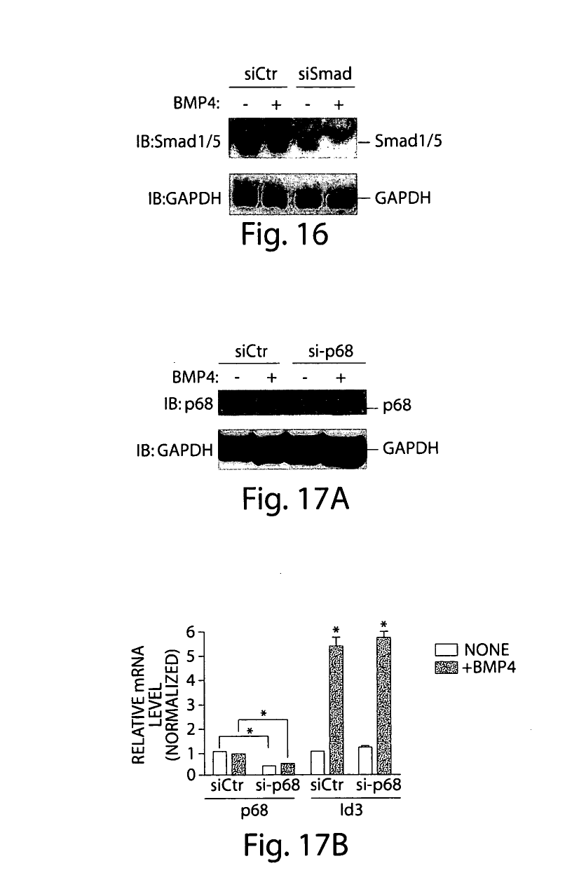

We investigated the molecular pathway leading to miR-21 induction by RNAi knockdown (˜80%, siSmads) of the BMP-specific R-Smad proteins expressed in PASMCs (Smad1 and Smad5) (FIG. 3a, bottom panel and FIG. 16). SiSmads abolished BMP4 induction of both pre-miR-21 and mature miR-21, while the level of expression of pri-miR-21 was not affected (FIG. 3a, top panel). Induction of SMA and of the BMP transcriptional target Id3 was also inhibited by Smad1/5 depletion, as expected (FIG. 3a, bottom panel). Therefore, R-Smads are required for pre-miR-21 stimulation by BMP4.

We postulated that the requirement of Smads for pre-miR-21 induction might entail a direct involvement of Smads in the Drosha microprocessor complex based on the previous report of a constitutive interaction between the carboxyl-terminal MH2 domain of Smad1 and the RNA helicase p68 [Warner, D. R. et al., Biochem Biophys Res Commun 324, 70-76 (2004)], a critical subunit of the Drosha microprocessor complex [Fukuda, T. et al., Nat Cell Biol 9, 604-611 (2007)]. To examine whether p68 is involved in the regulation of miR-21 expression by BMP4, p68 was reduced in PASMCs by siRNA (˜70%, FIG. 17). Expression of pri-miR-21 and the BMP4 target gene Id3 [Hollnagel, A., et al. J Biol Chem 274, 19838-19845 (1999)] did not change significantly (FIG. 17b), but induction of pre-miR-21 and mature-miR-21 by BMP4 was completely abolished (FIG. 3b), indicating an essential role of p68 in the TGFβ/BMP-regulated synthesis of pre-miR-21.

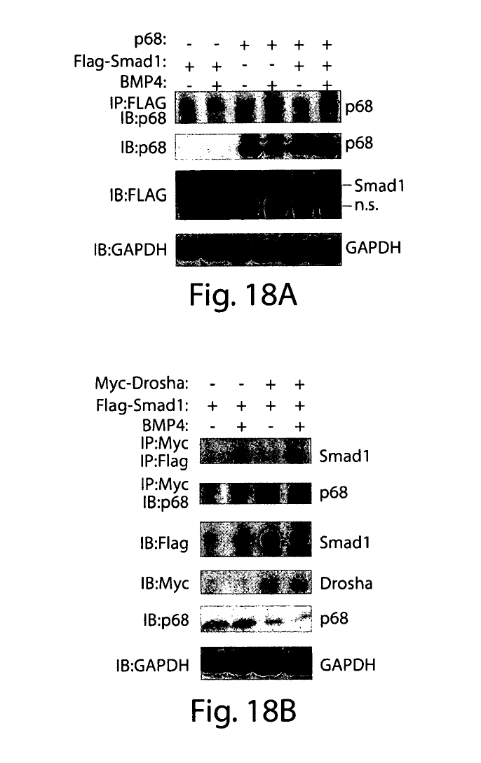

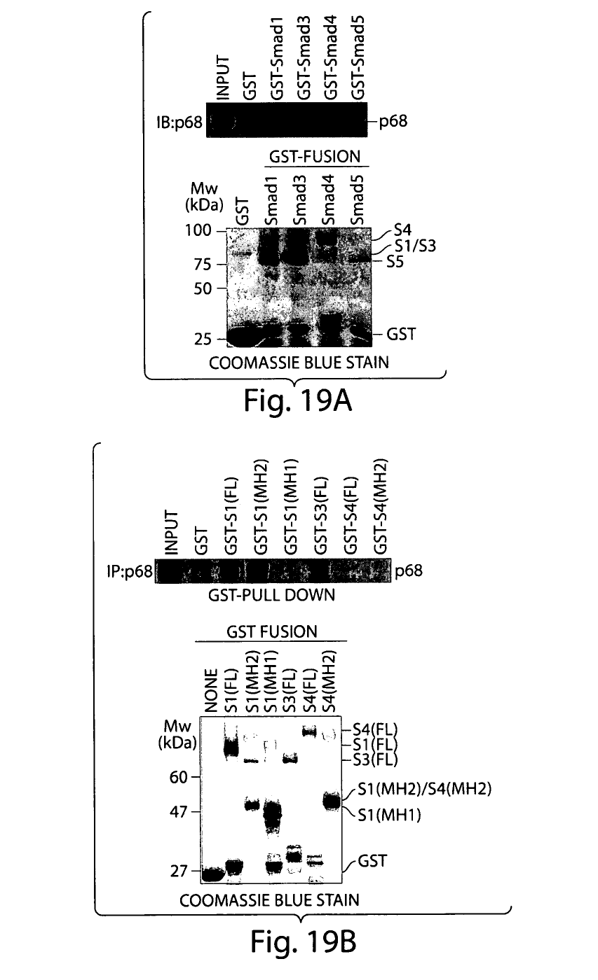

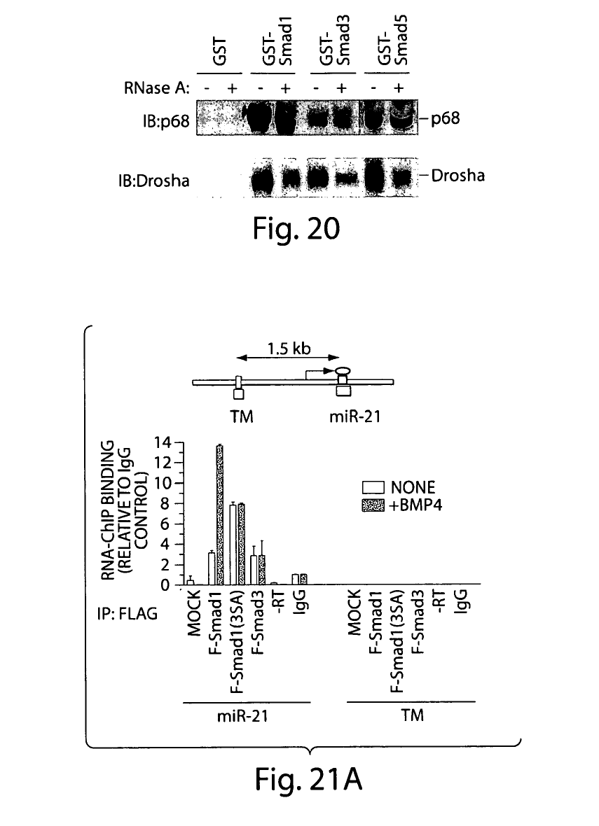

We found that the interaction between exogenous Smad1 and p68 is BMP4-inducible in Cos7 cells (FIG. 18a). In vitro (GST pull-down), p68 interacts both with BMP-specific Smad1 or Smad5 and with TGFβ-specific Smad3, suggesting that induction of pre-miR-21 by TGFβ may also involve a R-Smad/p68 complex (FIG. 19a). No interaction was observed between p68 and the cofactor Smad4 (FIG. 19a) or the inhibitor Smad6 (data not shown). The interaction between R-Smads and p68 was resistant to RNase A treatment, suggesting that R-Smads and p68 interact in the absence of pri-miRNAs (FIG. 20). We also confirmed that the carboxyl-terminal MH2 domain of Smad1 is sufficient to pull down p68 [Warner, D. R. et al., Biochem Biophys Res Commun 324, 70-76 (2004)], while the amino-terminal MH1 domain does not bind p68 (FIG. 19b). Thus, by binding p68, Smad1 may be recruited to the Drosha microprocessor complex. Indeed, upon BMP4 stimulation, Smad1 could be co-immunoprecipitated with Drosha from Cos7 extracts expressing tagged Drosha and Smad1 (FIG. 18b) or from PASMCs with endogenous proteins (FIG. 3c). The interaction of R-Smads with Drosha was markedly reduced by RNase A treatment (FIG. 20), suggesting that the association of R-Smads with Drosha, unlike the R-Smads/p68 complex, may be facilitated by miRNA transcripts. Therefore, following ligand stimulation, Smads associate with the Drosha microprocessor complex via interaction with p68, ultimately promoting accumulation of specific pre-miRNAs.

### Example 5

### Ligand-Inducible Association of R-Smads with Pri-miRNAs

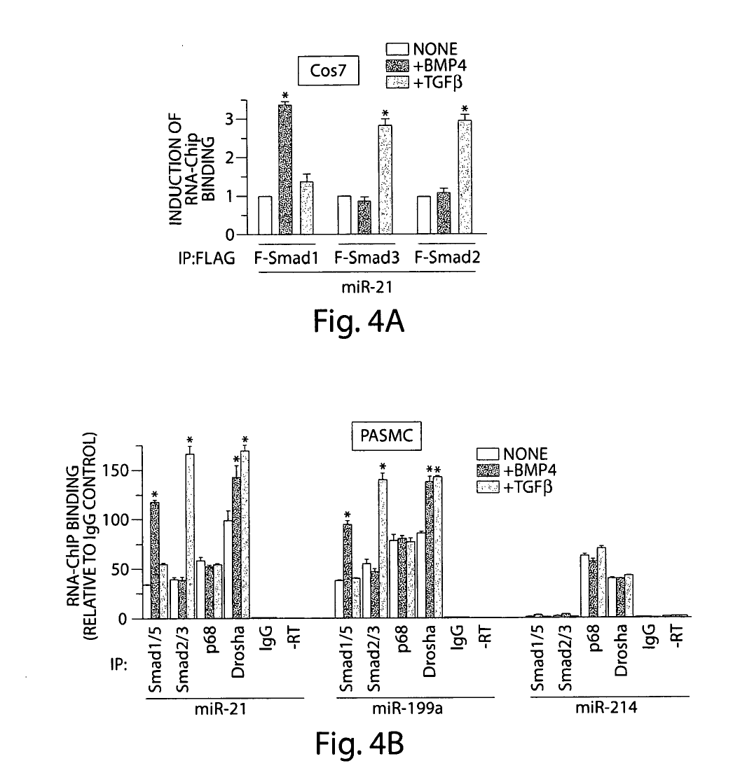

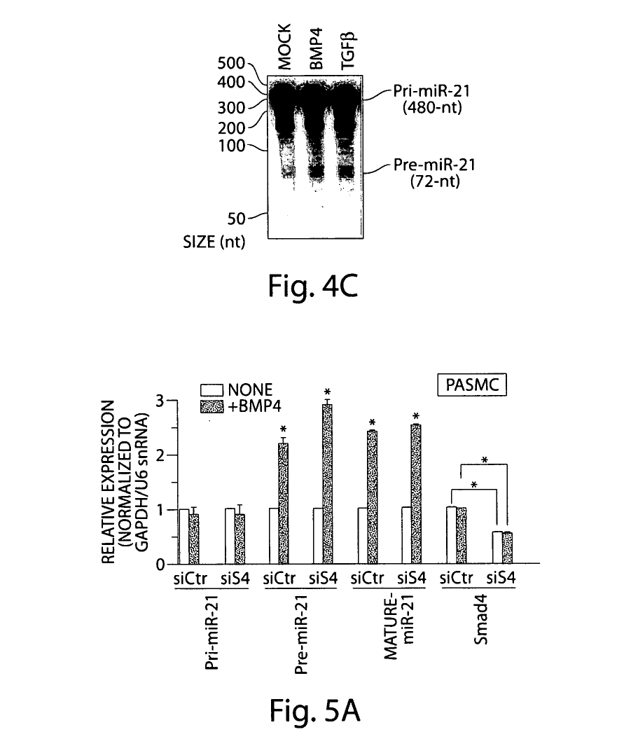

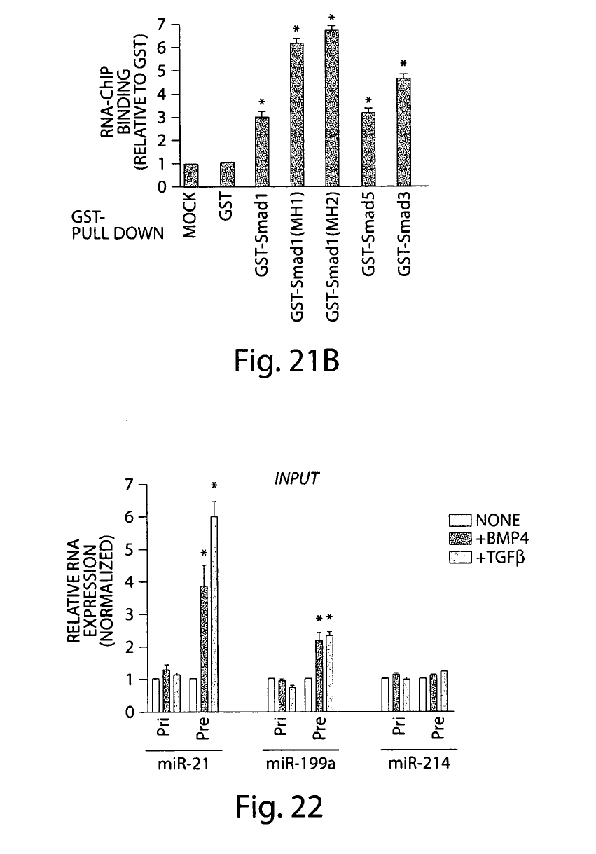

To test whether the Smad/p68/Drosha complex assembles specifically on pri-miR-21, we performed an RNA-ChIP analysis on Cos7 cells co-transfected with pCMV-miR-21 and Flag-tagged Smad1, Smad3 or Smad2. The association of Smad1 (but not Smad2 or Smad3) with pri-miR-21 was induced 3-fold upon BMP4 stimulation for 2 hr (FIG. 4a and FIG. 21a), while TGFβ increased binding to pri-miR-21 by Smad3 and Smad2, but not by Smad1, indicating that the association between R-Smads and pri-miR-21 is specifically regulated by ligands (FIG. 4a).

Endogenous Smads also interacted in a ligand-specific fashion with pri-miR-21 (FIG. 4b), while p68 constitutively associated with pri-miR-21 and the recruitment of Drosha was moderately enhanced by either TGFβ or BMP4 (FIG. 4b). Similar results were obtained for miR-199a (FIG. 4b). The significant increase we observed in the association of Drosha with pri-miR-21 and pri-miR-199a (FIG. 4b) suggests that binding of Smads to the pri-miRNA might stabilize the association between Drosha and the pri-miRNA. We detected a constitutive association of pri-miR-214 with p68 and Drosha, but no interaction with Smads (FIG. 4b), confirming that pre-miR-214 is not regulated by BMP or TGFβ signals (FIG. 22). Thus, recruitment of Smads to the p68/Drosha complex is pri-miRNA-specific.

A Smad1 mutant non-phosphorylatable upon BMP stimulation [Smad1 (3SA)] retained the ability to interact with pri-miR-21 (FIG. 21a). Furthermore, bacterially expressed unphosphorylated GST-Smad fusion proteins are able to interact with p68 (FIGS. 19 and 20), indicating that receptor-mediated phosphorylation of R-Smads is not essential for the association with pri-miRNA and suggesting that BMPs may affect the association between Smad1 and pri-miRNAs primarily by controlling Smad nuclear localization.

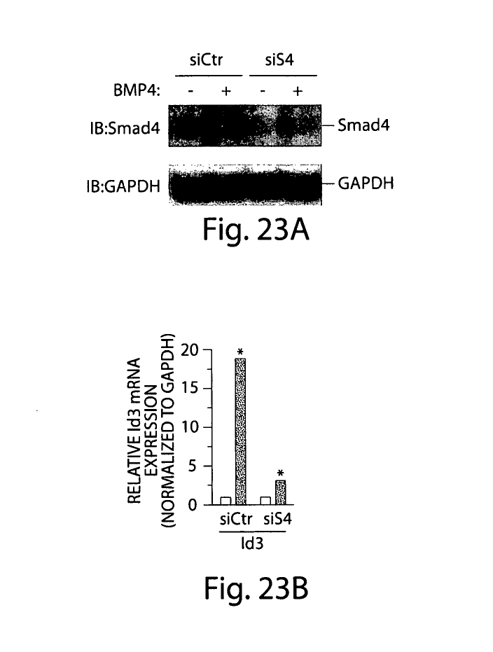

Pull-down experiments using partially purified GST-Smad fusion proteins as bait confirmed that Smad1, Smad3 and Smad5 can interact with pri-miR-21. Interestingly, both the MH1 and the MH2 domains of Smad1 bound to pri-miR-21 (FIG. 23). Since MH1 does not interact with p68 (FIG. 19b), it is possible that MH1 interacts either with pri-miR-21 directly or with other miR-21-binding proteins.

In summary, BMPs and TGFβ stimulate the expression of a specific subset of miRNAs by inducing the formation of a complex comprising ligand-specific Smad proteins, pri-miRNAs, and subunits of the microprocessor complex such as Drosha and p68.

Finally, we examined the possibility that ligand treatment may facilitate Drosha-mediated production of pre-miRNA. In vitro pri-miRNA processing assays were performed by incubating radiolabeled pri-miR-21 substrate (480-nt) with nuclear extracts from Cos7 cells treated with vehicle, BMP4 or TGFβ. Ligand treatment resulted in ˜25% increase (BMP4: 28.5%±1.9%; TGFβ: 24.2%±1.4%; triplicate experiments) in the production of a 72-nt product corresponding to pre-miR-21, compared to incubation with extracts from mock-treated cells (FIG. 4c). This result suggests that ligand-induced association of Smads with the Drosha complex increases pri-miR-21 cropping into pre-miRNA.

### Example 6

### Smad4-Independent Regulation of Maturation of miR-21

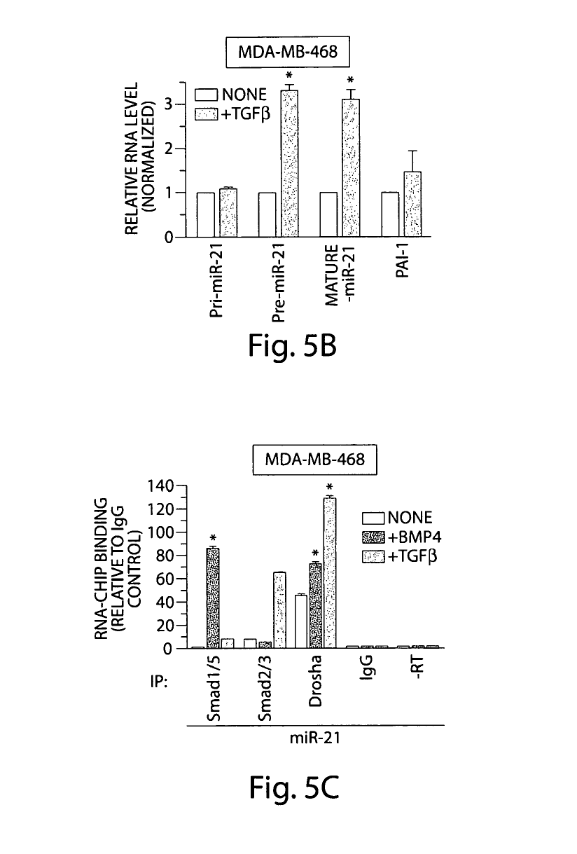

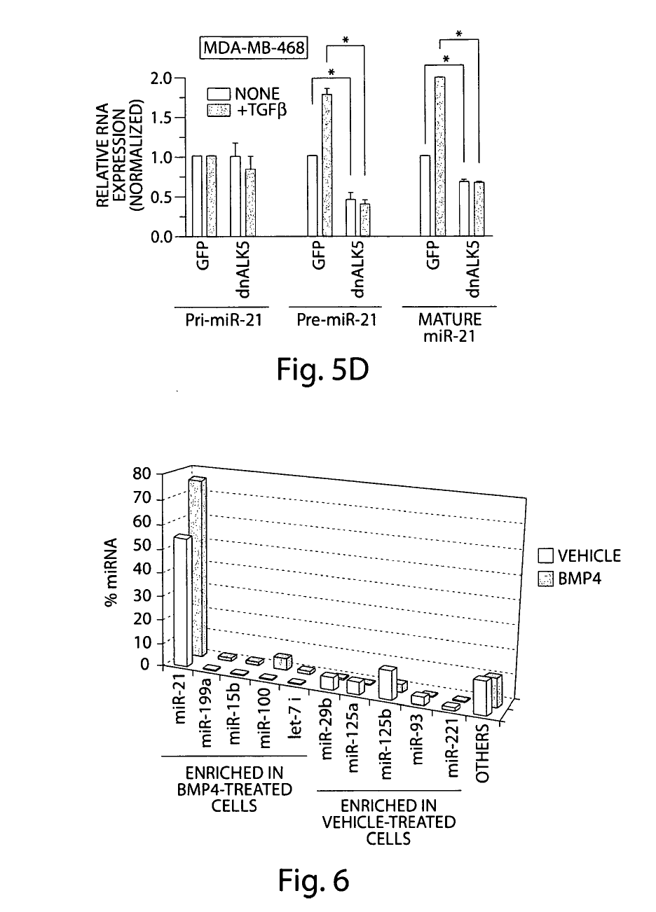

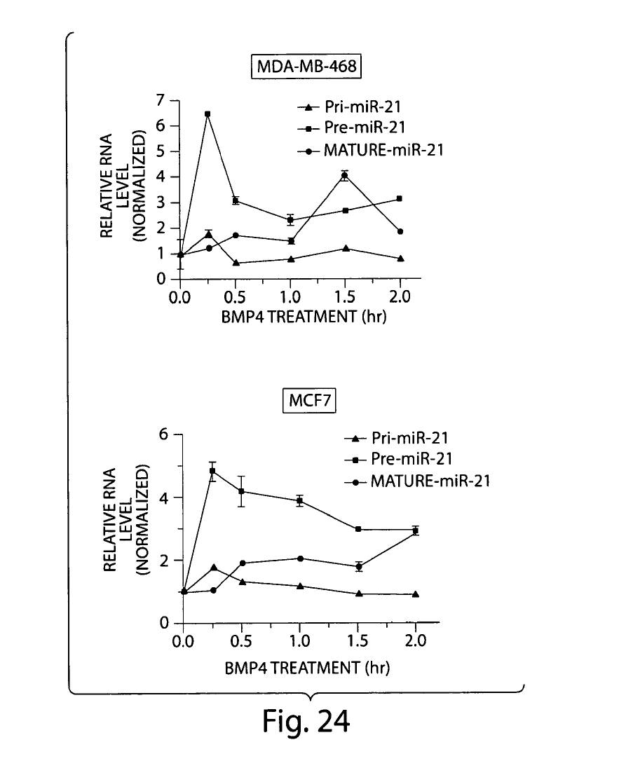

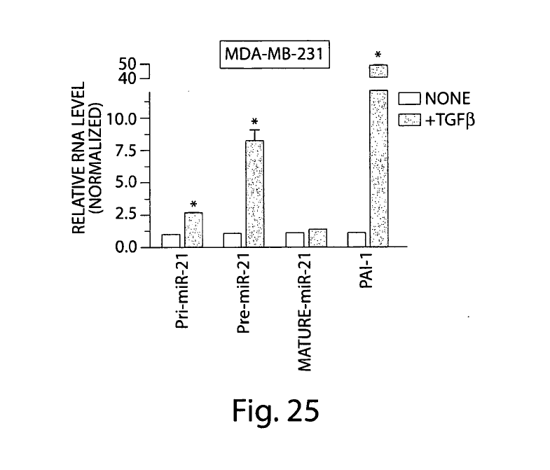

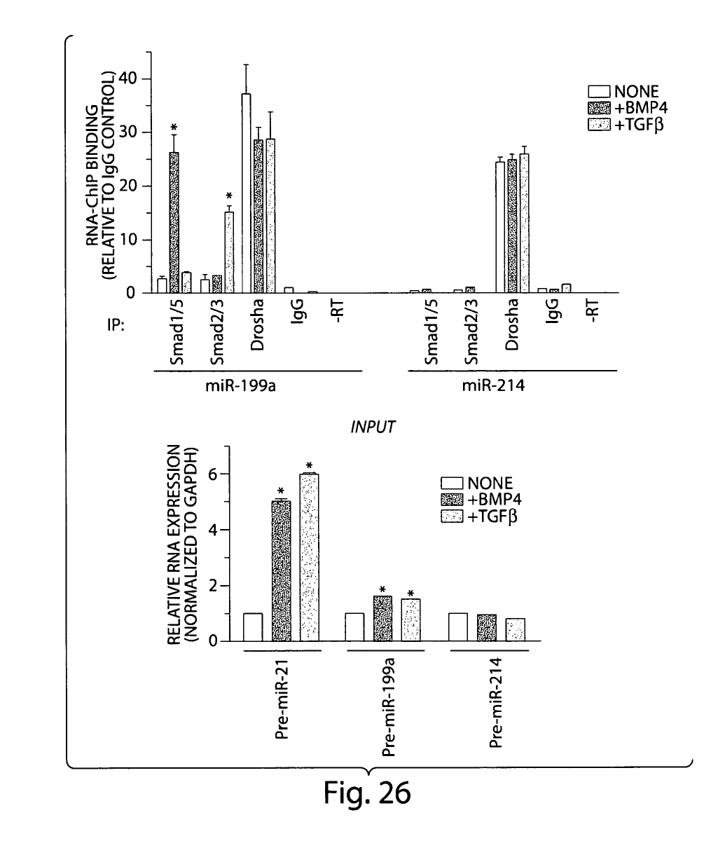

Two observations led us to speculate that Smad4 may be dispensable for the regulation of miR-21 processing: the lack of interaction between p68 and Smad4 (FIG. 19a), the co-Smad required for most transcriptional responses to BMP and TGFβ signaling; and the ability of the Smad1 (3 SA) mutant, which does not form a complex with Smad4 [Kretzschmar, M., et al. Genes Dev. 11, 984-995 (1997)], to associate with pri-miR-21 (FIG. 21a). Transfection of a siRNA against Smad4 (siS4) in PASMCs markedly reduced Smad4 protein (—90%, FIG. 23a) and RNA (FIG. 5a), as well as the transcriptional inducibility of the BMP target gene Id3 [Hollnagel, A., et al. J Biol Chem 274, 19838-19845 (1999)] (from 18-fold to 3-fold), as expected (FIG. 23b). However, siS4 did not affect the induction of pre-miR-21 or mature-miR-21 by BMP4 (FIG. 5a), in contrast with the result obtained from downregulation of R-Smads (FIG. 3a). Therefore, Smad4 is not required for the stimulation of processing of miR-21 by BMP4 in PASMCs. Cancer cells in which the canonical TGFβ pathway is impaired, as the Smad4-negative MDA-MB-468 cells, lack the ability to transcriptionally regulate a majority of TGFβ target genes [Gomis, R. R. et al., Proc Natl Acad Sci USA 103, 12747-12752 (2006) and Levy, L. & Hill, C. S., Mol Cell Biol 25, 8108-8125 (2005)] but retain some TGFβ responses, such as nuclear translocation of R-Smads, increased cell migration and epithelial-to-mesenchymal transition (EMT) [Levy, L. & Hill, C. S., Mol Cell Biol 25, 8108-8125 (2005), Giehl, K., et al. Cells Tissues Organs 185, 123-130 (2007), and Ijichi, H. et al., Oncogene 23, 1043-1051 (2004)]. We investigated whether miR-21 stimulation by TGFβ can still occur in MDA-MB-468 cells as it does in PASMCs depleted of Smad4. A rapid induction of pre-miR-21 and mature miR-21 was observed upon TGFβ stimulation in MDA-MB-468 cells, without change in the levels of pri-miR-21 or of the Smad4-dependent TGFβ target gene plasminogen activator inhibitor-1 (PAI-1) mRNA [Gomis, R. R. et al., Proc Natl Acad Sci USA 103, 12747-12752 (2006) and Levy, L. & Hill, C. S., Mol Cell Biol 25, 8108-8125 (2005)] (FIG. 5b). Similar results were obtained by BMP4 treatment of MDA-MB-468 and Smad4-expressing breast carcinoma MCF7 cells (FIG. 24) or TGFβ treatment of Smad4-positive breast carcinoma MDA-MB-231 cells (FIG. 25). Interestingly, stimulation of pri-miRNA processing by TGFβ does not necessarily lead to an increase in mature miRNA: unlike MDA-MB-468 cells (FIG. 5b), MDA-MB-231 cells display little elevation of mature miR-21 after TGFβ stimulation despite strong induction of pre-miR-21 (FIG. 25), suggesting the existence of another regulatory step of miRNA maturation after pri-miRNA cleavage by the Drosha microprocessor. An RNA-ChIP analysis confirmed that in MDA-MB-468 cells the association of R-Smads with the primary transcripts of miR-21 and miR-199a (but not miR-214) is ligand-inducible (FIG. 5c and FIG. 26). Therefore, Smad4 is not necessary for ligand-mediated processing of pri-miRNAs, and some of the Smad4-independent responses observed in ligand-stimulated cells may be mediated by regulation of miRNA biogenesis by the TGFβ or BMP pathways.

### Example 7

### TGFβ Signaling Plays a Role in the Increased Expression of Mature miR-21 in Breast Carcinoma

The expression of mature miR-21 is augmented in different types of tumors and tumor-derived cell lines, including breast carcinoma MCF-7, MDA-MB-231 and MDA-MB-468 cells [Frankel, L. B. et al., Programmed cell death 4 (PDCD4) is an important functional target of the microRNA miR-21 in breast cancer cells. J Biol Chem 283, 1026-1033 (2007), Si, M. L. et al., Oncogene 26, 2799-2803 (2007), Diederichs, S. & Haber, D. A., Cancer Res 66, 6097-6104 (2006), Volinia, S. et al., Proc Natl Acad Sci USA 103, 2257-2261 (2006), Iorio, M. V. et al., Cancer Res 65, 7065-7070 (2005), and Wiemer, E. A., Eur J Cancer 43, 1529-1544 (2007)]. Since TGFβ expression is often elevated in cancer cells, where it promotes EMT and metastatic behavior [Bierie, B. & Moses, H. L., Nat Rev Cancer 6, 506-520 (2006), Arteaga, C. L., Curr Opin Genet Dev 16, 30-37 (2006), Bachman, K. E. & Park, B. H., Curr Opin Oncol 17, 49-54 (2005), Glick, A. B., Cancer Biol Ther 3, 276-283 (2004), and Massague, J. & Gomis, R. R., FEBS Lett 580, 2811-2820 (2006)], we postulated that the increased levels of miR-21 may in part be due to autocrine TGFβ signaling. A dominant negative TGFβ type I receptor (dnALK5) [Fujii, M. et al., Mol. Biol. Cell 10, 3801-3813 (1999)], which harbors a mutation in the kinase domain, was expressed in MDA-MB-468 cells to inhibit TGFβ signaling. Both the basal and the TGFβ-induced expression of pre-miR-21 were greatly reduced, while the pri-miR-21 level was unchanged (FIG. 5d). These results indicate that autocrine TGFβ signaling contributes to the high basal expression of miR-21 in cancer cells.

This study underscores several unexpected findings. Firstly, the TGFβ/BMP family triggers VSMCs differentiation by increasing the expression of a subset of miRNAs. This induction occurs post-transcriptionally, likely at the level of processing of primary transcripts by the Drosha microprocessor complex. Ligand-specific Smad proteins bind to the Drosha microprocessor subunit p68 to facilitate pre-miRNA accumulation. Finally, we identified a novel mechanism by which the TGFβ pathway may promote the metastatic and invasive potential of cancer cells through modulation of biosynthesis of oncogenic miRNAs such as miR-21, which in turn targets tumor suppressor genes PDCD4 and tensin homolog deleted on chromosome 10 (PTEN) [Asangani, I. A. et al., Oncogene in press (2008) and Frankel, L. B. et al., J Biol Chem 283, 1026-1033 (2007)].

The MH1 domain of R-Smads binds DNA by specifically recognizing a sequence element [Massague, J., et al. Genes Dev. 19, 2783-2810 (2005) and Shi, Y. et al., Cell 94, 585-594 (1998)]; we observed that the MH1 domain of Smad1 associates with pri-miR-21 despite its inability to interact with p68. Smad MH1 domain may recognize an RNA sequence or structural element, and thus provide specificity in the selection of BMP/TGFβ target miRNA. Association of Smad with the Drosha complex is likely to contribute to various aspects of pri-miRNA processing, such as (i) facilitating the specific recognition and stable binding of Drosha to pri-miRNAs, (ii) increasing the RNase activity of Drosha, (iii) directing the cleavage of pri-miRNAs to a precise sequence, or (iv) modulating the stability of pre-miRNA. In summary, our findings open new avenues to the study of TGFβ-family signaling pathways and miRNA biogenesis regulation.

### Example 8

### Methods Summary

Cell culture. Cos7, C3H10T1/2, MDA-MB-468, MDA-MB-231, and MCF7 cells (American Type Culture Correction) were maintained in Dulbecco's Modified Eagle media (DMEM) supplemented with 10% fetal bovine serum (FBS, Sigma). Human primary pulmonary artery smooth muscle cells (PASMCs) were purchased from Lonza (CC-2581; on the web at lonzabioscience.com/Lonza_Catnay.oid.734.prodoid.PASMC) and were maintained in Sm-GM2 media (Lonza) containing 5 FBS.

Real-time RT-PCR. Total RNA was extracted by TRIzol (Invitrogen) and subjected to reverse transcription using first-strand cDNA synthesis kit (Invitrogen) according to the manufacturer's instructions. The quantitative analysis of the change in expression levels was calculated by real-time PCR machine (iQ5, Bio-Rad) [Schmittgen, T. D. et al., Methods 44, 31-38 (2008)]. For a detection of mature miRNAs, TaqMan MicroRNA assay kit (Applied Biosystems) was used according to manufacturer's instructions. Average of three experiments each performed in triplicate with standard errors of are presented.

Statistical Analysis. The results presented are average of at least three experiments each performed in triplicate with standard errors. Statistical analyses were performed by analysis of variance, followed by Tukey's multiple comparison test or by Student's t test as appropriate, using Prism 4 (GraphPAD Software Inc.). P values of <0.05 were considered significant and are indicated with asterisks.

RT-PCR primers. Human (SEQ ID NO: 20) pri-miR-21: 5′-TTTTGTTTTGCTTGGGAGGA-3′ and (SEQ ID NO: 21) 5′-AGCAGACAGTCAGGCAGGAT-3′. Human pre-miR-21: (SEQ ID NO: 22) 5′-TGTCGGGTAGCTTATCAGAC 3′ and (SEQ ID NO: 23) 5′-TGTCAGACAGCCCATCGACT-3′. (SEQ ID NO: 24) Human GAPDH: 5′-ACCACAGTCCATGCCATCAC-3′ and (SEQ ID NO: 25) 5′-TCCACCACCCTGTTGCTGTA-3′. Human SMA: (SEQ ID NO: 26) 5′-CCAGCTATGTGTGAAGAAGAGG-3′ and (SEQ ID NO: 27) 5′-GTGATCTCCTTCTGCATTCGGT-3′. Human Id1: (SEQ ID NO: 28) 5′-CCCATTCTGTTTCAGCCAGT-3′ and (SEQ ID NO: 29) 5′-TGTCGTAGAGCAGCACGTTT-3′. Human Id3: (SEQ ID NO: 30) 5′-ACTCAGCTTAGCCAGGTGGA-3′ and (SEQ ID NO: 31) 5′-AAGCTCCTTTTGTCGTTGGA-3′. Human PDCD4: (SEQ ID NO: 32) 5′-TATGATGTGGAGGAGGTGGATGTGA-3′ and (SEQ ID NO: 33) 5′-CCTTTCATCCAAAGGCAAAACTACA-3′. Human p68: (SEQ ID NO: 34) 5′-AGAGGTTCAGGTCGTTCCAGG-3′ and (SEQ ID NO: 35) 5′-GGAATATCCTGTTGGCATTGG-3′. Human calponin: (SEQ ID NO: 36) 5′-GAGTGTGCAGACGGAACTTCAGCC-3′ and (SEQ ID NO: 37) 5′-GTCTGTGCCCAGCTTGGGGTC-3′. Human SM22a: (SEQ ID NO: 38 5′-CGCGAAGTGCAGTCCAAAATCG-3′ and (SEQ ID NO: 39) 5′-GGGCTGGTTCTTCTTCAATGGGC-3′.

siRNAs. Synthetic siRNAs targeting human Smad1, Smad4, or Smad5 and p68 (DDX5) were Validated Stealth™ DuoPak (Invitrogen) and Stealth™ Select RNAi (Invitrogen), respectively. For Smad4: (SEQ ID NO: 40) 5′-CCUGAGUAUUGGUGUUCCAUUGCUU-3′ and (SEQ ID NO: 41) 5′-GCAAAGGUGUGCAGUUGGAAUGUAA-3′

### For Smad1: (SEQ ID NO: 42) 5′-GCAACCGAGUAACUGUGUCACCAUU-3′ and (SEQ ID NO: 43) 5′-GGUCUGCAUCAAUCCCUACCACUAU-3′.

### For Smad5: (SEQ ID NO: 44) 5′-GCCACCUGAUGAUCAGAUGGGUCAA-3′ and (SEQ ID NO: 45) 5′-GCUUGGGUUUGUUGUCAAAUGUUAA-3′.

For p68: (SEQ ID NO: 46) 5′-GGAAUCUUGAUGAGCUGCCUAAAUU-3′, (SEQ ID NO: 47) 5′-ACAACUGCCCGAAGCCAGUUCUAAA-3′, and (SEQ ID NO: 48) 5′-GGUGCAGCAAGUAGCUGCUGAAUAAA-3′. SiRNA for human PDCD4 was described¹¹ previously and synthesized by Dharmacon. As a negative control, Stealth™ RNAi Negative Control Duplex #1-3 (Invitrogen) or scrambled siRNA (Dharmacon) was used.

RNA-ChIP primers. Human miR-21: (SEQ ID NO: 20) 5′-TTTTGTTTTGCTTGGGAGGA-3′ and (SEQ ID NO: 21) 5′-AGCAGACAGTCAGGCAGGAT-3′. Human miR-199a: (SEQ ID NO: 49) 5′-GCCAACCCAGTGTTCAGACTA-3′ and (SEQ ID NO: 50) 5′-GCCTAACCAATGTGCAGACTA-3′. Human miR-214: (SEQ ID NO: 51) 5′-CCCTTTCCCCTTACTCTCCA-3′ and (SEQ ID NO: 52) 5′-CTATGGTGTGAGGGCTGCTT-3′. Human TM: (SEQ ID NO: 53) 5′-GCAAGCACATAGTGGAGCAA-3′ and (SEQ ID NO: 54) 5′-TCAAACATCCAGGACAACCA-3′.

Antibodies. Anti-Flag epitope tag (M2, Sigma), anti-p68 (clone PAb204, Upstate), anti-SMA (clone 1A4, Sigma), anti-Calponin (clone hCP, Sigma), anti-GAPDH (2E3-2E10, Abnova), anti-Smad2/Smad3 (#06-654, Upstate), anti-Smad1/Smad5/Smad8 (Calbiochem), anti-Smad4 (H-552, Santa Cruz), anti-Myc epitope tag (clone 9E10, Tufts antibody core facility), anti-Lamin-A/C (#2032, Cell Signaling), and anti-Drosha (#07-717, Upstate) antibodies.

In vitro pri-miRNA processing assays. In vitro pri-miRNA processing assay was performed as previously described [Guil, S. & Caceres, J. F., Nat Struct Mol Biol 14, 591-596 (2007)]. Briefly, the 480-nt radiolabeld pri-miR-21 was prepared by standard in vitro transcription with T7 RNA polymerase in the presence of [α-³²P]-UTP using human miR-21 gene cloned into pGEM-3 vector as a template. Nuclear extracts were prepared from ˜5×10⁶ Cos7 cells treated with vehicle, 400 pM TGFβ or 3 nM BMP4 for 2 h. After dialysis into reaction buffer, nuclear extracts were incubated with pri-miR-21 substrates for 90 min at 37° C. Reaction mixtures were subjected to phenol-chroloform extraction, precipitation and 10% (w/v) denaturing gel electrophoresis, followed by autoradiography. The amount of pri-miR-21 (input) and pre-miR-21 was quantitated by the phosphoimager (Typhoon9410, GE Healthcare) using ImageQuant 350 software (GE Healthcare).

MiRNA and cDNA Expression Constructs. pCMV-miR-21 construct and recombinant adenovirus carrying miR-21 or miR-125b (Ad-miR-21 or Ad-miR-125b) were reported previously [van Rooij, E. et al., Proc Natl Acad Sci USA 103, 18255-18260 (2006) and Zhu, S., et al. J Biol Chem 282, 14328-14336 (2007). Briefly, pCMV-miR-21 construct contains 480 by human miR-21 genomic fragments cloned into a modified pCMV-Myc vector (Clontech). Ad-miR-21 and Ad-miR-125b contain 280 by rat miR-21 and 366 by miR-125b genomic fragments into CMV-driven adenoviral vector, respectively. To monitor the amount of pri-miR-21 and pre-miR-21 derived from the pCMV-miR-21 construct in murine 10T1/2 cells, human-specific RT-PCR primers complementary to sequences in the miR-21 flanking region were used. Unlike pri-miR-21 or pre-miR-21, mature miR-21, which is identical in mouse and human, was detected as the sum of the endogenous and recombinant products. Human PDCD4 and p68 cDNA construct were purchased from OriGene. Briefly, a full-length human PDCD4 cDNA with 1.9 kb 3′-UTR (NM_{—}01445), which contains miR-21 target sequence, is cloned into pCMV6 vector. Human Drosha cDNA construct was purchased from Addgene. Flag-Smad1 (3SA) construct (a gift from Massague lab) contains human Smad1 cDNA mutated from Ser to Ala mutations at a.a. 462, 463, and 465 and cloned into pCMV5 vector [Kretzschmar, M., et al. Genes Dev. 11, 984-995 (1997)].

Plasmid DNA and siRNA Transfection. Cos7, 10T1/2 cells, or PASMCs were transfected with FuGENE 6 (Roche Applied Science) for plasmid DNAs and Oligofectamine (Invitrogen) for siRNAs as described before [Chan, M. C. et al., Mol Cell Biol 27, 5776-5789 (2007)].

Adenoviral Infection. The recombinant adenoviruses were generated and purified by standard procedures. Infection of adenoviruses was performed at 100 multiplicity of infection (M.O.I.). There was no detectable toxicity to the cells under these conditions.

qRT-PCR assays. For qRT-PCR assays, total RNA was extracted from cells by TRIzol (Invitrogen). cDNA was synthesized from 1 μg of purified RNA by SuperScript II First-Strand cDNA synthesis system (Invitrogen) according to manufacturer's instructions. qRT-PCR was performed with a real-time PCR machine (iQ5, Bio-Rad). The results of qRT-PCR assays presented are average of three independent RNA preparations. Each sample was analyzed in triplicate. PCR cycling parameters were: 94° C. for 3 min, and 40 cycles of 94° C. for 15 s, 60° C. for 20 s, 72° C. for 40 s). For detection of mature miRNAs, TaqMan MicroRNA assay kit (Applied Biosystems) was used according to manufacturer's protocol. Data analysis was done by using the comparative C_{T} method in software by Bio-Rad.

Luciferase assay. After transfection of the reporter construct together with LacZ plasmid as internal control, the cells were reseeded onto 12-well plates and treated with 3 nM BMP3 or 400 pM TGFβ1 for 16-20 h in DMEM/0.2% FCS. Luciferase assays were carried out using Promega's Luciferase assay system. Luciferase activity was normalized with LacZ activity.

Anti-miRNAs. 2′-O-methyl modified RNA oligonucleotides complementary to miRNA (anti-miR) or GFP (control) sequence were purchased from IDT. Anti-miRs were transfected to cells at a concentration of 106 nM using Oligofectamine (Invitrogen) according to manufacturer's directions. Anti-miR-21: (SEQ ID NO: 55) 5′-GUCAACAUCAGUCUGAUAAGCUA-3′. Anti-miR-199a: (SEQ ID NO: 56) 5′-GAACAGGUAGUCUGAACACUGGG-3′. Anti-miR-125b: (SEQ ID NO: 57) 5′-UCACAAGUUAGGGUCUCAGGGA-3′. Anti-miR-221: (SEQ ID NO: 58) 5′-GAAACCCAGCAGACAAUGUAGCU-3′. Anti-miR-15b: (SEQ ID NO: 59) 5′-UGUAAACCAUGAUGUGCUGCUA-3′. Anti-miR-100: (SEQ ID NO: 60) 5′-CACAAGUUCGGAUCUACGGGUU-3′. Anti-GFP: (SEQ ID NO: 61) 5′-AAGGCAAGCUGACCCUGAAGU-3′.

miRNA cloning. miRNA cloning from PASMCs was performed following the protocol from David Bartel lab (Whitehead Institute for Biomedical Research). Briefly, miRNAs were prepared from PASMCs treated with 3 nM BMP4 for 24 h using Trizol (Invitrogen). After linker ligation and PCR amplification, miRNA sequences were concatemerized, cloned into Topo-TA vector, and sequenced by Tufts Core Facility.

RNA-ChIP. Was performed as previously described [Fukuda, T. et al., Nat Cell Biol 9, 604-611 (2007)]. Briefly, PASMCs or Cos7 cells were crosslinked for 15 minutes with 1% formaldehyde, the cell pellet was resuspended in Buffer A (5 mM PIPES, pH 8.0, 85 mM KCl, 0.5% Nonidet P-40). After 10 min on ice, the crude nuclei fraction was isolated by centrifugation, and then suspended in Buffer B (1% SDS, 10 mM EDTA, 50 mM Tris-HCl, pH 8.1). After nuclei were disrupted by sonication, the lysates were cleared and subjected to immunoprecipitation with anti-Flag, anti-Smad1/5/8, Smad2/3, or p68 antibody, prior to stringent washing, and elution. The RNA was isolated using Trizol (invitrogen). Pellets were resuspended in 100 μl TE and incubated with DNase I (10 U) for 30 min at 37° C. to remove any remaining DNA. Following extraction with phenol:chloroform (5:1), RNA was precipitated with ethanol and dissolved in 20 μl of water. 5 μA of RNA was used for a 20 μl cDNA synthesis reaction. Quantitative PCR reactions were then performed by real-time PCR machine (iQ5, Bio-Rad). Average of three experiments each performed in triplicate with standard errors are presented.

GST pull-down assay. GST-Smad fusion proteins were expressed in bacteria, followed by partial purification with GST-sepharose beads. Equal amounts of GST-Smad fusion proteins conjugated to sepharose beads were added to nuclear extracts or total RNA. After washing the beads, proteins pulled-down with the beads were separated on a SDS-PAGE, followed by immunoblotting or RT-PCR analysis. For RNase A treatment, 250 μg/ml RNase A (New England Biolab) were added to nuclear extracts 30 min prior to addition of GST-Smad fusion proteins and throughout the pull-down assay.

Immunoprecipitation/Immunoblot assay. Cells were lysed in TNE buffer (1% Nonidet P-40, 10 mM Tris-HCl (pH 7.5), 1 mM EDTA, 150 mM NaCl). Total cell lysates or proteins immunoprecipitated with antibodies were separated on a SDS-PAGE, transferred to PVDF membranes (Millipore), immunoblotted with antibodies, and visualized using an enhanced chemiluminescence detection system (Amersham Biosciences).

Immunofluorescence staining. PASMCs or 10T1/2 cells were fixed and permeabilized in a 50% acetone-50% methanol solution and subjected to staining using anti-SMA or anti-calponin antibody conjugated with fluorescein isothiocyanate (FITC) and nuclear staining with 4′-6′-Diamidino-2-phenylindole (DAPI, Invitrogen).

RNA Interference. Synthetic small interference RNA (siRNA) targeting human Smad1, Smad4, Smad5, or p68 were obtained from Invitrogen (Validated Stealt™ DuoPak). A siRNA with a nontargeting sequence (scramble siRNA, Dharmacon) was used as a negative control. The siRNAs were transfected as described before 4. d nuclear staining with 4′-6-Diamidino-2-phenylindole (DAPI, Invitrogen).

### Example 9

### microRNAs with CAGAC Box Sequence Mature miRNA

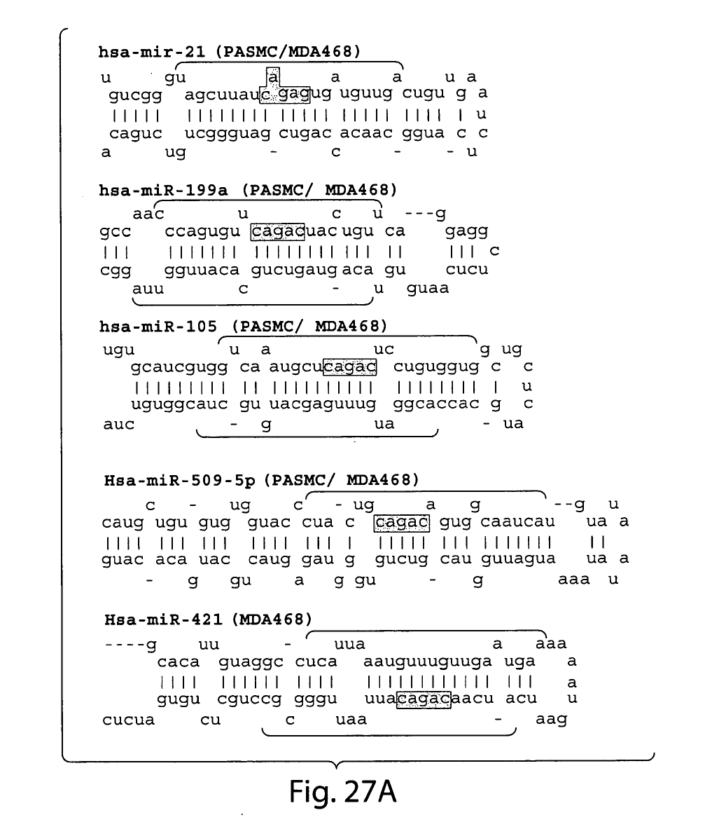

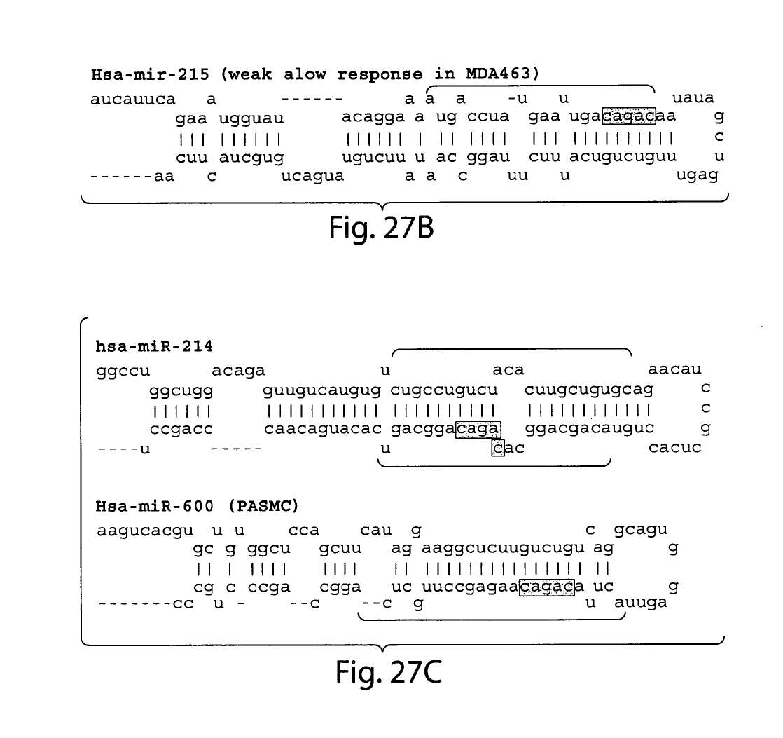

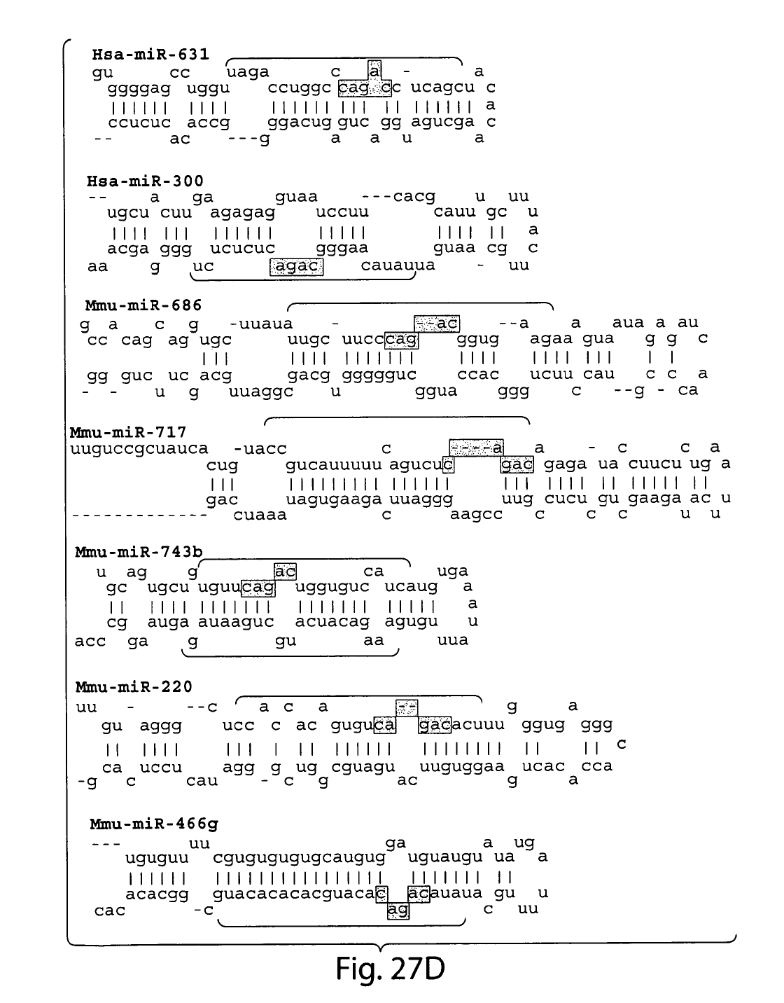

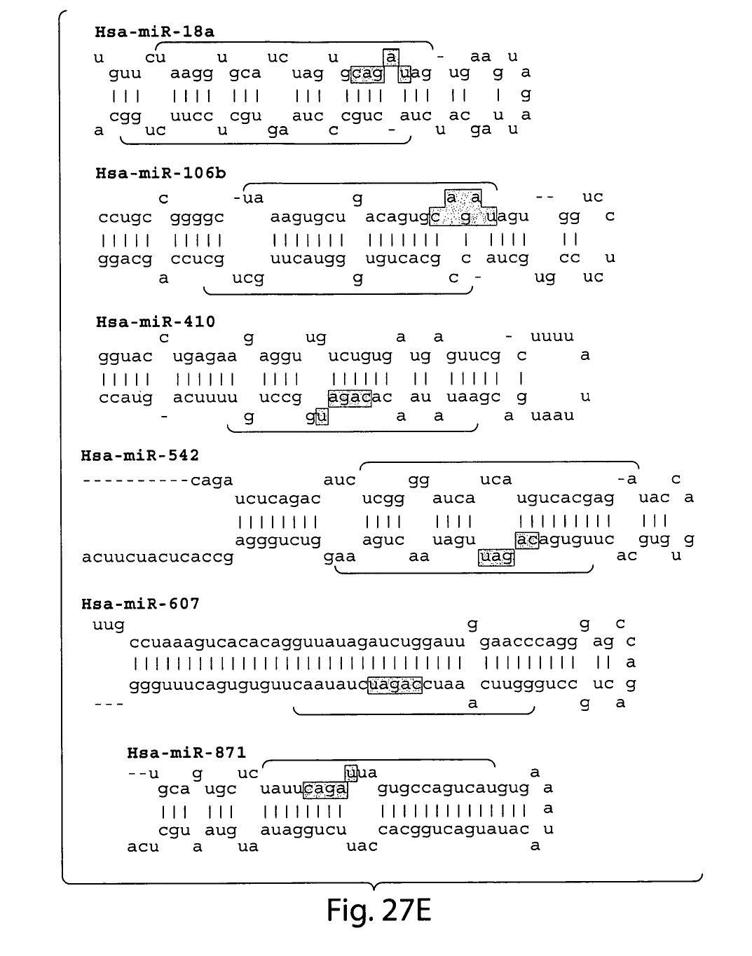

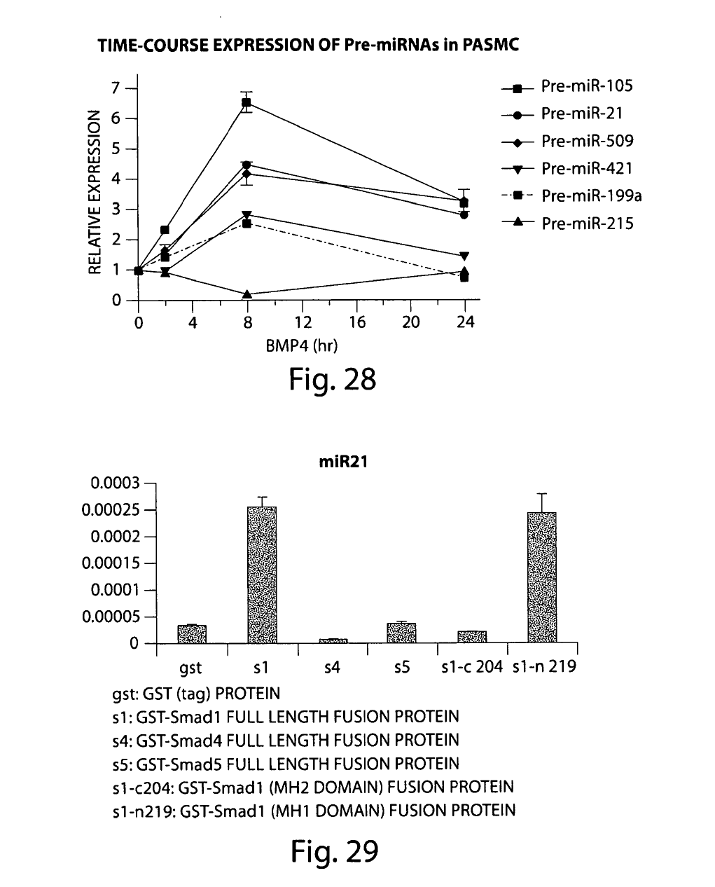

We combined computational sequence searching of miRNA databases with visual inspection of sequences to identify miRNAs containing CAGAC sequences (i.e., TGF miRNAs). We identified TGF miRNAs Hsa-mir-21 (SEQ ID NO: 62), Hsa-miR-199a (SEQ ID NO: 63), Hsa-miR-105 (SEQ ID NO: 65), Hsa-miR-509-5p (SEQ ID NO: 66), Hsa-miR-421 (SEQ ID NO: 69), which are strongly regulated by TGFβ/BMP in PASMC and/or MDA468 cells. Hsa-miR-215 (SEQ ID NO: 70), (weak slow response in MDA468) was identified and found to be weakly regulated by TGFβ/BMP in MDA468 cells. Hsa-miR-214 (SEQ ID NO: 71), and Hsa-miR-600 (SEQ ID NO: 72), were identified and not found to be regulated by TGFβ/BMP in PASMC and MDA468 cells. Hsa-miR-631 (SEQ ID NO: 158), Hsa-miR-300 (SEQ ID NO: 159), Mmu-miR-686 (SEQ ID NO: 160), Mmu-miR-717 (SEQ ID NO: 161), Mmu-miR-743b (SEQ ID NO: 162), Mmu-miR-220 (SEQ ID NO: 163), and Mmu-miR-466g (SEQ ID NO: 164), were identified but not tested for TGFβ/BMP regulation. Hsa-miR-18a (SEQ ID NO: 165), Hsa-miR-106b (SEQ ID NO: 166), Hsa-miR-410 (SEQ ID NO: 167), Hsa-miR-542 (SEQ ID NO: 168), Hsa-miR-607 (SEQ ID NO: 169), and Hsa-miR-871 (SEQ ID NO: 170), were identified as microRNAs with CAGAT (an alternative SMAD binding element (SBE)). These CAGAT microRNAs were identified but not tested for TGFβ/BMP regulation. Additional viral miRs with CAGAC were identified but not tested; these include mghv-miR-M1-2, ebv-miR-BART-11-5p, and rlcv-miR-rL1-12-5p. We examined the stem-loop (hairpin) structure of these miRNAs containing CAGAC sequences in the region of their mature miRNA (FIG. 27.) The sequences below are exemplary microRNAs with CAGAC box sequence. The Mature miRNA is underlined and SMAD Binding Element (SBE) sequence is shown in italics. We evaluated the expression of TGF miRNAs following TGFβ/BMP treatment in human pulmonary artery smooth muscle cells (PASMC) and human breast cancer cells (MDA-468) (see Table 1 and FIG. 28).

>hsa-mir-21 MI0000077
(SEQ ID NO: 62)
UGUCGGGUAGCUUAU [symbol] UGAUGUUGACUGUUGAAUCUCAUGGCAACACCAGUCGAUG
GGCUGUCUGACA
>hsa-mir-199a-1 MI0000242
(SEQ ID NO: 63)
GCCAACCCAGUGUU [symbol] UACCUGUUCAGGAGGCUCUCAAUGUGUACAGUAGUCUGCACA
UUGGUUAGGC
>hsa-mir-199a-2 MI0000281
(SEQ ID NO: 64)
AGGAAGCUUCUGGAGAUCCUGCUCCGUCGCCCCAGUGUUCAGACUACCUGUUCAGGACAAU
GCCGUUGUACAGUAGUCUGCACAUUGGUUAGACUGGGCAAGGGAGAGCA
>hsa-mir-105-1 MI0000111
(SEQ ID NO: 65)
UGUGCAUCGUGGUCAAAUGCU [symbol] UCCUGUGGUGGCUGCUCAUGCACCACGGAUGUUU
GAGCAUGUGCUACGGUGUCUA
>hsa-mir-509-1 MI0003196
(SEQ ID NO: 66)
CAUGCUGUGUGUGGUACCCUACUG [symbol] AGUGGCAAUCAUGUAUAAUUAAAAAUGAUUG
GUACGUCUGUGGGUAGAGUACUGCAUGACACAUG
>hsa-mir-509-2 MI0005530
(SEQ ID NO: 67)
CAUGCUGUGUGUGGUACCCUACUG [symbol] AGUGGCAAUCAUGUAUAAUUAAAAAUGAUUG
GUACGUCUGUGGGUAGAGUACUGCAUGACAC
>hsa-mir-509-3 MI0005717
(SEQ ID NO: 68)
GUGGUACCCUACUG [symbol] GUGGCAAUCAUGUAUAAUUAAAAAUGAUUGGUACGUCUGUG
GGUAGAGUACUGCAU
>hsa-mir-421 MI0003685
(SEQ ID NO: 69)
GCACAUUGUAGGCCUCAUUAAAUGUUUGUUGAAUGAAAAAAUGAAUCAUCAA [symbol] AUU
AAUUGGGCGCCUGCUCUGUGAUCUC
>hsa-mir-215 MI0000291
(SEQ ID NO: 70)
AUCAUUCAGAAAUGGUAUACAGGAAAAUGACCUAUGAAUUGA [symbol] AAUAUAGCUGAGU
UUGUCUGUCAUUUCUUUAGGCCAAUAUUCUGUAUGACUGUGCUACUUCAA
-----------------------------------------------------------------------
>hsa-mir-214 MI0000290
(SEQ ID NO: 71)
GGCCUGGCUGGACAGAGUUGUCAUGUGUCUGCCUGUCUACACUUGCUGUGCAGAACAUCC
GCUCACCUGUACAGCAGGCA [symbol] AGGCAGUCACAUGACAACCCAGCCU
>hsa-mir-600 MI0003613
(SEQ ID NO: 72)
AAGUCACGUGCUGUGGCUCCAGCUUCAUAGGAAGGCUCUUGUCUGUCAGGCAGUGGAGUU
ACUUA [symbol] AAGAGCCUUGCUCAGGCCAGCCCUGCCC

Mature TGF miRNA Sequences:

| (SEQ ID NO: 73) | |
|---|---|
| UAGCUUAU [symbol] UGAUGUUGA | miR-21 |
| (SEQ ID NO: 74) | |
| CCCAGUGUU [symbol] UACCUGUU | miR-199A |
| (SEQ ID NO: 75) | |
| UCAAAUGCU [symbol] UCCUGUGGU | miR-105 |
| (SEQ ID NO: 76) | |
| CUACUG [symbol] AGUGGCAAUCA | miR-509-1 |
| (SEQ ID NO: 77) | |
| AUCAA [symbol] AUUAAUUGGGCGC | miR-421 |
| (SEQ ID NO: 78) | |
| AUGACCUAUGAAUUGA [symbol] AAUAUA | miR-215 |
| (SEQ ID NO: 79) | |
| ACAGCAGGCA [symbol] AGGCAGU | miR-214 |
| (SEQ ID NO: 80) | |
| ACUUA [symbol] AAGAGCCUUGCUC | miR-600 |
| (SEQ ID NO: 81) | |
| UAGACCUGGCC [symbol] CUCAGC | miR-631 |
| (SEQ ID NO: 82) | |
| UAUACAAGGG [symbol] UCUCUCU | miR-300 |

TABLE 1
Expression of TGF miRNAs following TGFβ/BMP treatment in human
pulmonary artery smooth muscle cells (PASMC) and human breast
cancer cells (MDA-468).

| | | | | | miR-509-1 | | | | |
|---|---|---|---|---|---|---|---|---|---|
| Cell | | miR-21 | miR-199a | miR-105 | (5p) | miR-421 | miR-215 | miR-214 | miR-600 |
| type | Ligand | U [symbol] U | U [symbol] U | U [symbol] U | G [symbol] A | A [symbol] A | A [symbol] A | A [symbol] A | A [symbol] A |
| PASMC | TGF | 6 fold | 7.5 fold | 3 fold | 4 fold | No | 4 fold | No | No |
| | | (2 hr) | (2 hr) | (2 hr) | (2 hr) | | (2 hr) | | |
| | | | | | | | 5 fold | | |
| | | | | | | | (4 hr) | | |
| | BMP | 3 fold | 2.5 fold | 8 fold | 3 fold | 2.5 fold | 1.5 fold | No | No |
| | | (2 hr) | (2 hr) | (2 hr) | (2 hr) | (8 hr) | (2 hr) | | |
| MDA- | TGF | 4 fold | Not | 13 fold | 23 fold | 10 fold | 3 fold | No | Not |
| 468 | | (0.5 hr) | Texted | (0.5 hr) | (0.5 hr) | (0.5 hr) | (0.5 hr) | | Tested |
| | | 2.5 fold | | 3 fold | 10 fold | | 4 fold | | |
| | | (1 hr) | | (1 hr) | (1 hr) | | (1 hr) | | |
| | BMP | 6 fold | Not | 10 fold | 40 fold | 25 fold | 5.5 fold | No | Not |
| | | (0.25 hr) | Tested | (1 hr) | (0.5 hr) | (1 hr) | (1 hr) | | Tested |
| | | 3 fold | | | 28 fold | | | | |
| | | (1 hr) | | | (1 hr) | | | | |

### Example 10

### SMAD Proteins Interact with Double Stranded CAGAC Sequences of miRNA

To examine the interaction of SMAD proteins with double stranded CAGAC sequences of miRNA we performed RNA pull down experiments. Gst-Smad fusion proteins were expressed in E. coli and partially purified. As a proof of principal, we used mature miR-21 sequences (see below). ssRNA oligonucleotides were annealed to make dsRNA. GST-Pull down experiments were performed using the standard methods including aspects of those disclosed herein in the presence of excess amount of non-specific RNAa (yeast tRNAs). Results are shown in FIG. 29. Full length GST-SMAD1 fusion protein and a GST-SMAD1 N-terminal (n219) MH1 domain interact with double stranded CAGAC sequences of miR-21

mature miR-21 sequences dsRNA sequence
miR-21 (SEQ ID NO: 73) 5′-UAGCUUAUCAGACUGAUGUUGA-3′
miR-21 (SEQ ID NO: 83) 3′-AUCGAAUAGUCUGACUACAACU-5′

### Example 11

### Smad Proteins Bind a Conserved RNA Sequence to Promote microRNA Maturation by Drosha

Smad proteins, the Transforming Growth Factor β (TGF β)/Bone Morphogenetic Protein (BMP) signal transducers, promote the expression of a subset of miRNAs by facilitating the first cleavage reaction by the Drosha microprocessor complex. The mechanism that limits Smad-mediated processing to a selective group of miRNAs remained hitherto unexplored. In this study, we expand the number of miRNAs regulated post-transcriptionally by TGFβ and BMP signaling. Surprisingly, these miRNAs contain a consensus sequence identical to the optimal DNA sequence bound by Smads in the promoters of TGF13/BMP target genes. We demonstrate that Smads specifically bind this sequence element (R-SBE) within the double-stranded stem region of primary miRNA transcripts. Mutation of the R-SBE abrogates TGFβ-induced recruitment of Smads, Drosha, and DGCR8 to pri-miRNAs, and impairs their processing. Thus, Smads are multifunctional proteins which modulate gene expression transcriptionally through DNA binding, and post-transcriptionally by pri-miRNA binding and regulation of processing.

Mature miRNAs are noncoding RNA molecules of ˜21-25 nucleotides (nt) in length. miRNAs regulate gene expression by targeting mRNAs, as single stranded molecules, in a sequence-specific manner and triggering several potential outcomes, including translational repression or mRNA degradation (Bartel, 2004; Berezikov and Plasterk, 2005; Cullen, 2006; Kim, 2005; Kim and Nam, 2006; Mallory and Vaucheret, 2006). The sequence of many miRNAs is conserved between distantly related organisms, suggesting that these molecules participate in fundamental biological processes (Niwa and Slack, 2007). Indeed, many miRNAs are involved in the regulation of gene expression during development, cell proliferation, apoptosis, glucose metabolism, and stress resistance. Aberrant miRNA expression is associated with various developmental abnormalities and human diseases, including cardiovascular disorders and cancer. Therefore, it is essential not only to dissect the individual enzymatic steps that create miRNAs, but also to discover the vital nodes of regulation in this process.

miRNAs are initially transcribed by RNA polymerase II as long primary transcripts, known as pri-miRNAs, containing both a 5′ cap and a poly(A) tail. Pri-miRNA is processed in the nucleus by the RNase III enzyme Drosha, which releases a hairpin shaped precursor miRNA (pre-miRNA) of ˜65-70 nt. Pre-miRNA is then exported to the cytoplasm where it undergoes the second processing step by the RNase III Dicer, completing the generation of a ˜22 nt mature miRNA-miRNA* duplex. The mature microRNA is incorporated into the RNA-induced silencing complex (RISC) where it mediates silencing of target genes (Carthew and Sontheimer, 2009; Kim et al., 2009; Siomi and Siomi, 2009).

The biogenesis of miRNA appears regulated at multiple steps in response to physiological stimuli, and the mechanisms involved are starting to be outlined (Calin and Croce, 2006; Cullen, 2006; Hammond, 2006; Taganov et al., 2007; Wiemer, 2007; Wienholds and Plasterk, 2005). The genomic regions encoding miRNAs display the defining features of the promoters of protein coding genes, such as specific histone modifications, CpG islands, TATA box, transcription initiator elements, and transcription factor binding sites (Ozsolak et al., 2008). This similarity, which hints to a transcriptional regulation of miRNA biogenesis, has been corroborated by the unveiling of several tissue-specific transcription factors controlling miRNA expression (e.g., c-Myc (Coller et al., 2007), serum response factor (SRF), myocyte enhancer factor 2 (MEF2), Myf-5, Myo-D, myogenin (van Rooij et al., 2008), C/EBP and PU.1 (Fukao et al., 2007).

The first processing step, catalyzed by Drosha, takes place concurrently with or shortly after transcription of the pri-miRNA (Morlando et al., 2008). Drosha is part of a large “microprocessor” complex, which includes regulatory subunits such as DGCR8 (also known as Pasha) and RNA helicases p68 or p72. A typical metazoan pri-miRNA consists of a 33-bp stem, a terminal loop, and ssRNA flanking segments. Drosha transiently interacts with the pri-miRNA stem, cleaving at ˜11 by from the ssRNA-dsRNA junction to generate a ˜65-70 nt pre-miRNA. DGCR8, which can directly interact with pri-miRNA, assists this process by correctly positioning and anchoring Drosha to the pri-miRNA. The exact role of p68 or p72 in the Drosha microprocessor complex is less clear, but we know from a gene deletion study that p68 and p72 are required for the biogenesis of a subset of miRNAs (Fukuda et al., 2007). Other proteins may also interact with Drosha or the pri-miRNA, with varying degree of specificity. For instance, Lin28 and nuclear ribonucleoprotein (hnRNP) A1 have been shown to bind to the terminal loop region of pri-let-7 and pri-miR-18a, respectively, and alter cleavage by the Drosha microprocessor complex (Guil and Caceres, 2007; Michlewski et al., 2008; Rybak et al., 2008; Viswanathan et al., 2008). Conversely, proteins of the NF-90 and NF-45 family inhibit pri-miRNA processing by Drosha (Sakamoto et al., 2008). Although it is still unclear how many pri-miRNAs are regulated at the level of the first cleavage step and the number of proteins involved, it seems that the relative low efficiency of this step makes it amenable to modulation by auxiliary factors. Post-transcriptional control of miRNA biogenesis at the level of Dicer processing can also occur. For example, despite similar expression of their precursor forms, levels of mature miR-143 and miR-145 are significantly lower in colorectal tumor compared to normal samples, implying an altered processing by Dicer in cancer tissue as cause of differential expression (Michael et al., 2003). It was also proposed that Lin28 may inhibit the processing of pre-let-7 in the cytoplasm either by blocking Dicer cleavage or by inducing the terminal uridylation and degradation of pre-let-7 (Heo et al., 2008; Rybak et al., 2008; Wulczyn et al., 2007).

We have shown that TGFβs and BMPs, two subgroups of factors within the TGFβ family, mediate rapid induction of miRNA (miR)-21 and miR-199a in human primary pulmonary smooth muscle cells (PASMCs). TGFβs and BMPs post-transcriptionally regulate the expression of miR-21 and miR-199a, promoting the processing of pri-miRNA into pre-miRNA by the Drosha/DGCR8 microprocessor complex in the nucleus (Davis et al., 2008). We observed that R-Smads, the transducers of TGFβ and BMP signals, translocate to the nucleus in response to ligand stimulation, associate with the large p68/Drosha/DGCR8 microprocessor complex and facilitate the cleavage of pri-miRNA to pre-miRNA by Drosha. We provide herein further information regarding the molecular mechanism that singles out certain miRNAs, such as miR-21 and miR-199a for regulation by the TGF β/-Smad pathway.

We identified that an expanded set of miRNAs, including miR-21 and miR-199a, are regulated post-transcriptionally by TGFβ and BMP signaling (Davis et al., 2008). A striking feature unites these miRNAs: the stem region of their primary transcripts contains a highly conserved sequence identical to the DNA Smad binding element (D-SBE) found in the promoters of TGFβ/BMP regulated genes (Massague et al., 2005). We demonstrate that R-Smads directly associate with this RNA SBE (R-SBE) through the amino-terminus MH1 domain. Mutations in the R-SBE abolish the TGFβ/BMP-mediated induction of pre-miRNA synthesis and impair pri-miRNA binding to Drosha and DGCR8 in vivo. Altogether, these results indicate that sequence-specific association of R-Smads to pri-miRNAs provides a platform for Drosha and DGCR8 docking and mediates more efficient cleavage by Drosha. Versatile nucleic acid recognition by Smad proteins provides a mechanism of selection and regulation of a set of pri-miRNAs by the TGFβ/BMP signaling pathway.

### Identification of Novel miRNAs Regulated by R-Smads

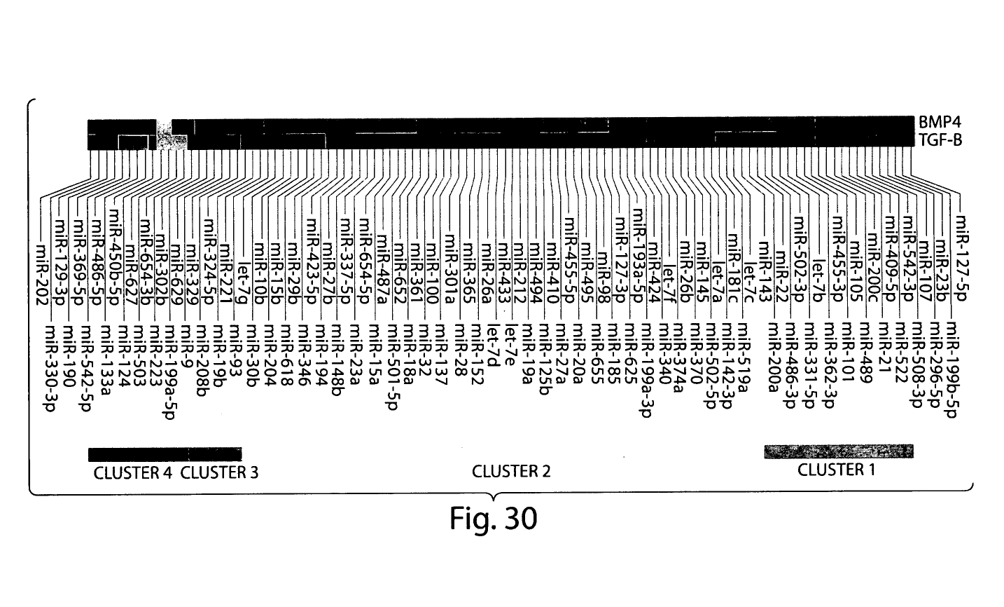

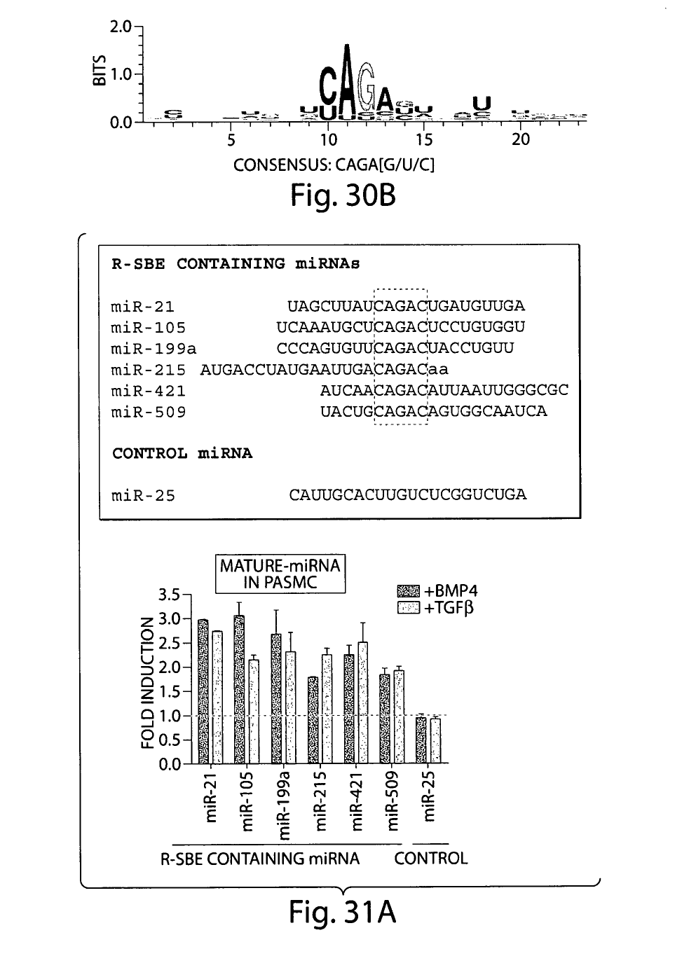

To identify novel miRNAs regulated by R-Smads similarly to miR-21 and miR-199a (Davis et al., 2008), we performed a miRNA microarray profiling analysis (TaqMan microRNA assays by Applied Biosystems) using human primary pulmonary artery smooth muscle cells (PASMCs) stimulated with BMP4 or TGFβ for 24 hr. Approximately 5% of the miRNAs analyzed (20 out of 377), including miR-21, were induced more than 1.6-fold by both BMP4 and TGFβ (FIG. 30A, Cluster 1). In contrast to the large number of miRNAs upregulated by BMP4 or TGFβ, few miRNAs were repressed by these growth factors (FIG. 30A, Cluster 4). Because we previously identified miR-21 and miR-199a as increased by both TGFβ and BMP4 in a smad4-independent manner, we focused on those microRNAs induced by both growth factors. Interestingly, detection of over-represented motifs within the sequences of the 20 mature miRNAs in Cluster 1 revealed the presence of a common sequence (in 18 out of 20) with the consensus “CAGAB” (5′-CAGA[C/G/U]-3′), identical to the D-SBE found in the promoter region of TGF target genes, such as plasminogen activator inhibitor type-1 (PAI-1), TGFβ1, α2(I) collagen, and germline Iga constant region (Dennler et al., 1998; Massague et al., 2005). On the contrary, none of the miRNAs that were not significantly regulated by TGFβ or BMP4 in the microarray analysis contained a “CAGAB” sequence (0 out of 38). Finally, searching a database of human conserved miRNAs for the “CAGAB” sequence yielded 81 miRNAs, the majority of which (35 out of 39 present on the array) are upregulated by TGFβ and/or BMP4 in the microarray analysis (Table1, shown in red). Altogether, these results indicate that the “CAGAB” sequence, which is an RNA-SBE (R-SBE), is frequently found among miRNAs regulated by either TGFβ or BMP4 signaling.

Post-Transcriptional Regulation by TGFβ/BMP4 of miRNAs Containing R-SBE

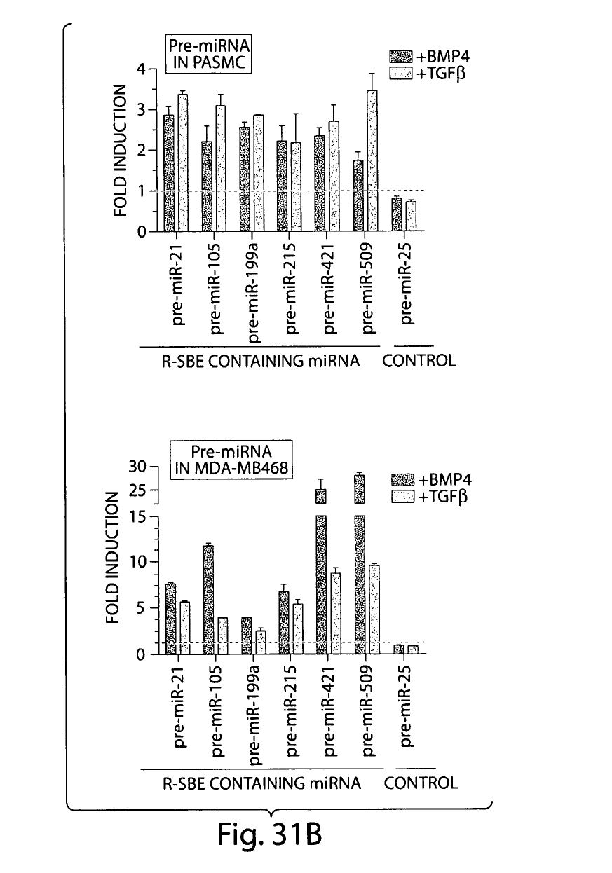

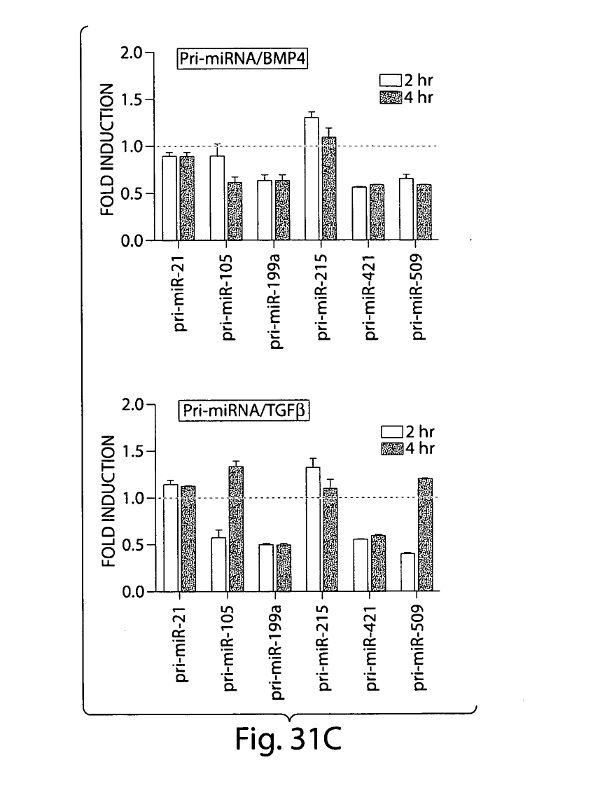

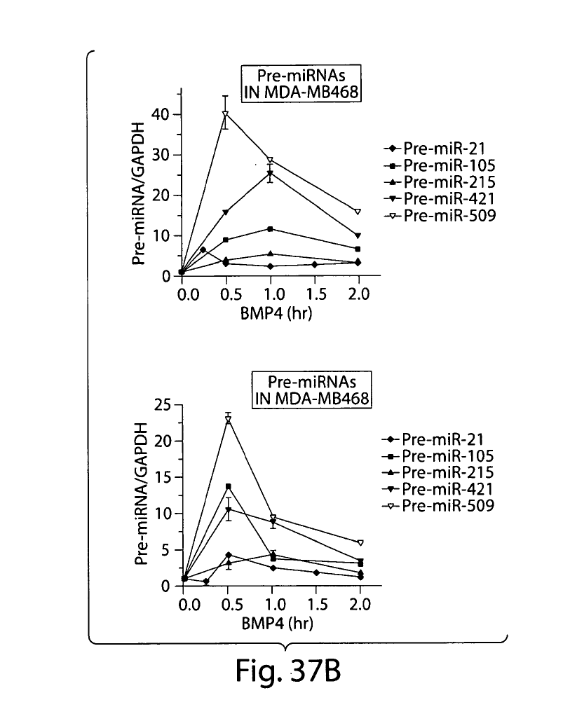

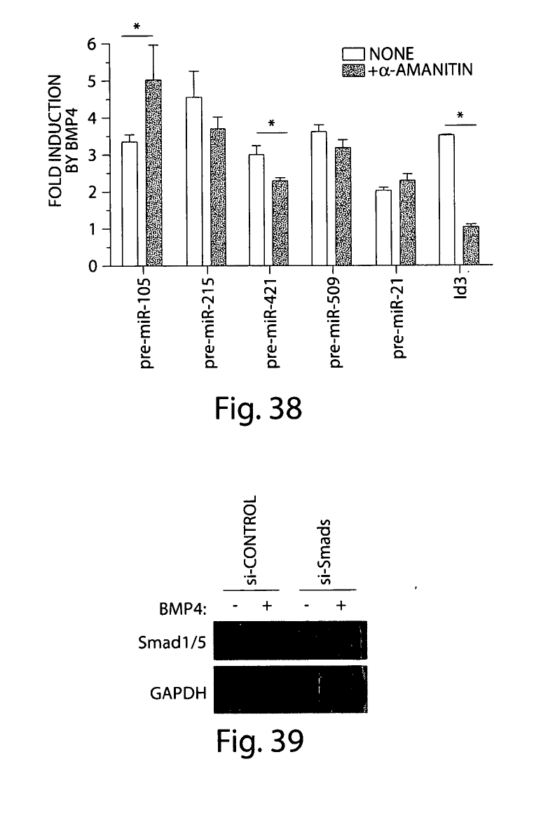

We selected 6 miRNAs (R-SBE miRNAs, including miR-21 and -199a) that contain a narrowly defined R-SBE sequence (5′-CAGAC-3′), and studied their expression after TGFβ and BMP4 stimulation in PASMCs (FIG. 31A). miR-25, which is not regulated by TGFβ or BMP4 and does not contain an R-SBE, was tested as control (FIG. 31A). qRT-PCR analysis confirmed that these six R-SBE miRNAs are rapidly induced 2-to-4-fold within 6 hr of TGFβ or BMP4 treatment in PASMCs (FIG. 31A). The pre-miRNA form of the R-SBE miRNAs (R-SBE pre-miRNAs) was induced upon TGFβ or BMP4 treatment within 6 hr in PASMC (FIG. 31B, PASMC) and in the human breast carcinoma cell line MDA-MB-468, which has a deletion in the gene encoding Smad4, an essential cofactor for transcriptional regulation by R-Smads (FIG. 31B, MDA-MB-468). All R-SBE pre-miRNAs were rapidly induced by TGFβ or BMP4 in PASMC and MDA-MB-468 cells, suggesting that the induction of R-SBE miRNAs is Smad4-independent and likely to occur post-transcriptionally (Davis et al., 2008). A time course study indicated that induction of pre-miRNAs, both in PASMC and MDA-MB-468 cells, is generally rapid, with a significant increase observed as early as 2 hr after stimulation (FIG. 37). To inquire whether some of the R-SBE miRNAs are also transcriptionally regulated by TGFβ or BMP4 signaling, we examined the expression of the primary transcripts of R-SBE miRNAs (R-SBE pri-miRNAs). R-SBE pri-miRNAs were not significantly increased after 2 or 4 hr of TGFβ or BMP4 treatment and prior to induction of mature miRNA at 6 hr. (FIG. 31C). Rather, the majority of the R-SBE pri-miRNA levels were decreased upon TGFβ or BMP4 stimulation (FIG. 31C and FIG. 37), suggesting that a rapid induction of processing from primary to precursor miRNA causes a transient reduction of pri-miRNAs. Induction of pre-miRNAs by BMP4 was also resistant to the RNA pol II transcription inhibitor α-amanitin, while induction of the transcriptional target of BMP4 Id3 (Korchynskyi and ten Dijke, 2002) was completely abolished under the same conditions (FIG. 38). Thus, we confirm that the TGFβ/BMP4 pathway regulates post-transcriptionally all the R-SBE miRNAs examined (Davis et al., 2008).

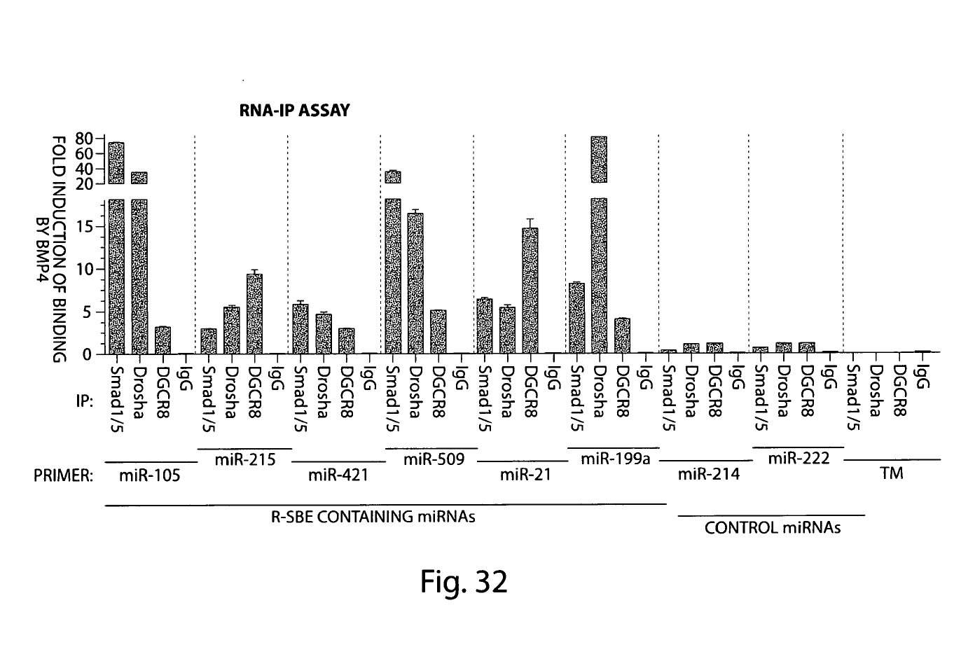

An RNA-immunoprecipitation (RNA-IP) assay indicated that BMP4 strongly induces the recruitment of R-Smads, Drosha, and DGCR8 to the primary transcripts of R-SBE miRNAs (FIG. 32). BMP4 treatment did not alter recruitment of R-Smads or Drosha/DGCR8 to transcripts of miRNAs that are not regulated by TGFβ signaling, such as miR-214 and miR-222 (FIG. 32), establishing a correlation between presence of an R-SBE in primary miRNA transcripts and the ability to recruit R-Smads and Drosha in response to TGFβ.

R-Smads Facilitate Recruitment of Drosha to R-SBE-Containing pri-miRNAs

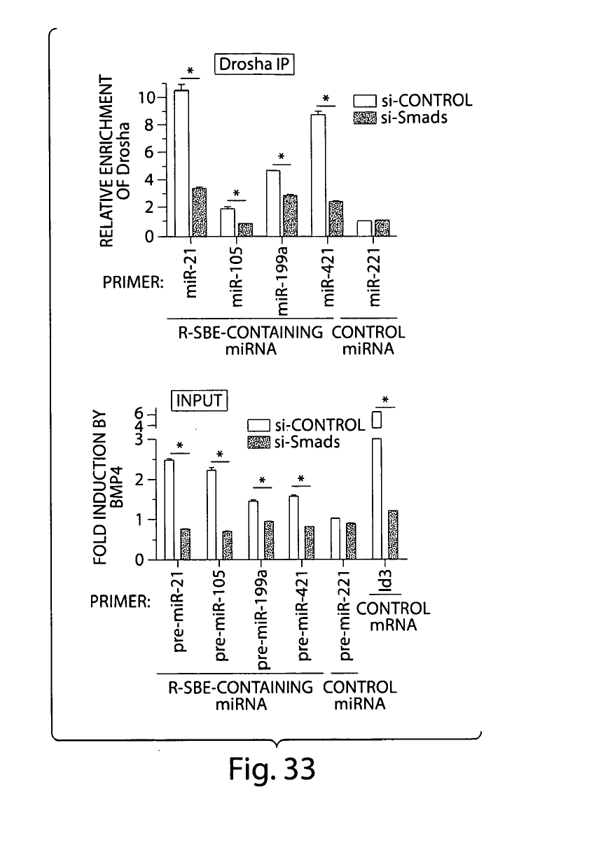

To examine whether R-Smads are specifically required for recruitment of Drosha to R-SBE pri-miRNAs, we knocked down endogenous BMP-specific R-Smads (Smad1 and Smad5) by siRNA (si-Smads) in PASMC (>95% reduction, FIG. 39). BMP4 treatment did not increase expression of the Id3 gene or of any of the R-SBE pre-miRNAs in si-Smads-transfected cells (FIG. 33, bottom panel, Input). Smad1/5 knockdown also resulted in a significant reduction of Drosha recruitment to the transcripts of R-SBE miRNAs (FIG. 33, top panel, Drosha IP), but had no effect on recruitment of Drosha to miR-221, which is not regulated by TGFβ/BMP4 (FIG. 33, top panel, Drosha IP). Therefore, R-Smads are required for ligand-induced recruitment of Drosha to R-SBE pri-miRNAs.

### R-SBE is Critical for the TGFβ/BMP-Dependent Processing by Drosha

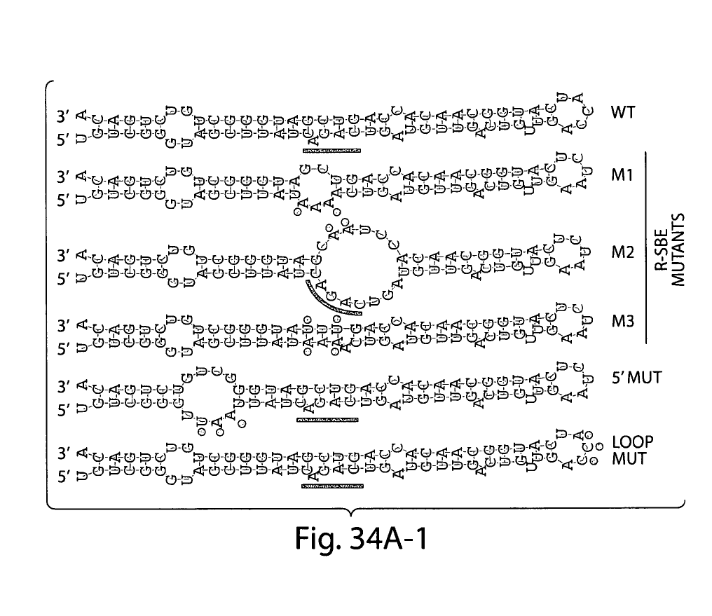

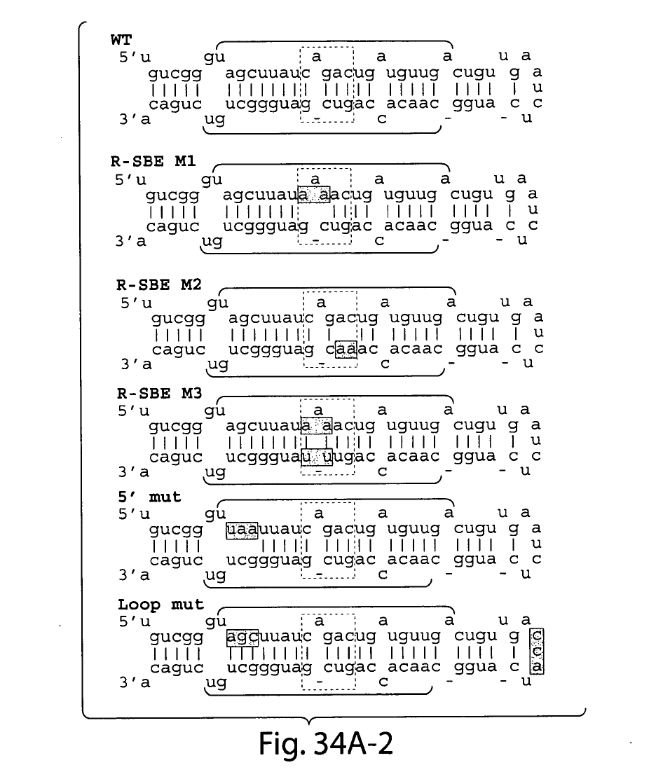

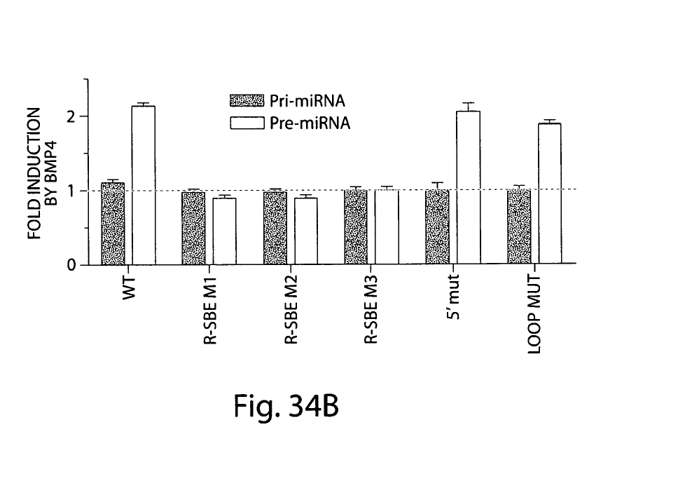

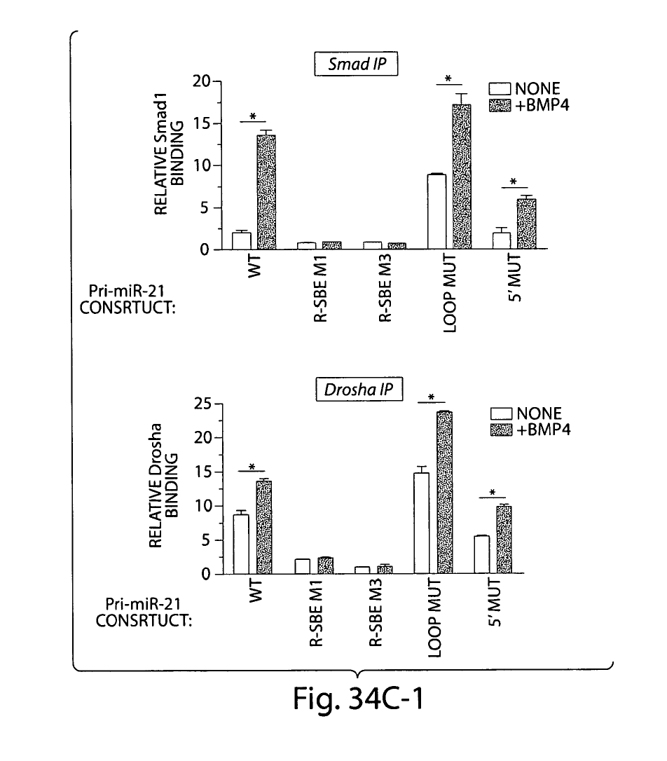

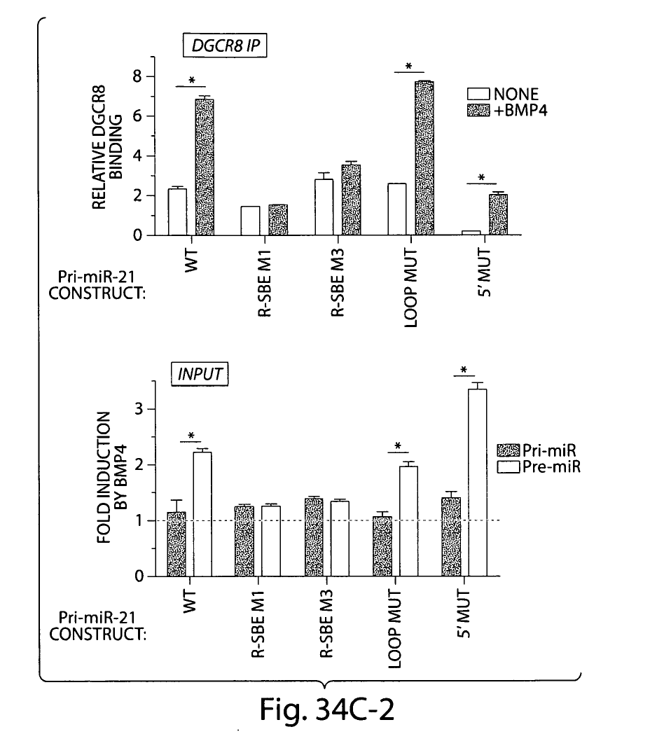

To test if an intact R-SBE in a TGFβ/BMP-regulated miRNA is necessary for recruitment of Drosha and pri-miRNA processing, we generated 2-3 by mutations in an expression construct for human pri-miR-21 (˜150 bp). The mutations targeted the R-SBE (M1-M3 mutants), the terminal loop region enclosed in the hairpin structure of pre-miR-21 (Loop mutant), and a sequence upstream of the R-SBE in the stem region (5′ mutant) (FIG. 34A). BMP-dependent processing of these mutants was examined in mouse C3H10T1/2 cells to allow specific detection of exogenous human pri-miR-21 transcripts (FIG. 34B). Induction of pre-miR-21 and mature miR-21 was completely abolished when the R-SBE sequence was mutated, suggesting that R-SBE is indeed essential for the TGFβ/BMP-dependent induction of pre-miRNA and mature miRNA (FIG. 34B). The M3 mutant in particular has the R-SBE sequence disrupted (from CAGA to AAAA), but conserves the double-stranded (ds) stem structure in the hairpin region. Its failure to respond to BMP stimulation suggests that the stem structure is not sufficient for BMP/TGFβ regulation (FIG. 34B). Unlike the R-SBE mutants, triple nucleotide mutations in the terminal loop region or in the sequence adjacent to the R-SBE did not significantly alter the BMP-dependent cropping of pri-miRNA (FIG. 34B, “Loop mut” and “5′ mut”). Altogether, these results demonstrate that the R-SBE sequence is critical for ligand-dependent induction of pri-miRNA processing of TGFβ/BMP-regulated miRNAs (FIG. 34B).

Consistent with the result of in vivo processing (FIG. 34B), RNA-IP analysis indicated that Smad1 was recruited upon BMP4 treatment to wild type (WT) pri-miR-21, as well as “Loop mut” or “5′ mut” (FIG. 34C, Smad IP). Both the basal and the BMP4-induced recruitment of Smad1 to the R-SBE mutants (M1 and M3) were significantly decreased in comparison with WT, “Loop mut” or “5′ mut” (FIG. 34C, Smad IP). Thus, the R-SBE is required for ligand-induced recruitment of Smad proteins to the R-SBE miRNAs. Similar to Smad1, both Drosha and DGCR8 were recruited to WT or Loop mut in a BMP4-dependent manner, but not to the R-SBE mutants (FIG. 34C, Drosha IP and DGCR8 IP). Together with the observation that downregulation of Smads inhibits ligand-dependent recruitment of Drosha to the TGFβ/BMP-regulated miRNAs (FIG. 33), these results confirm that ligand-induced recruitment of Smad to the R-SBE is essential for the ligand-induced recruitment of the Drosha/DGCR8 microprocessor complex and subsequent cropping of the pri-miRNAs. In contrast, the terminal loop and the 5′ region sequences are not critical for the ability of ligands to induce miRNA processing and Smad1, Drosha and DGCR8 binding, but do somewhat affect the basal levels of these measurements, suggesting that the alterations in RNA secondary structure can alter basal recruitment of the minimal Drosha/DGCR8 complex (FIG. 34C).

### Smad MH1 Domain Binds to R-SBE

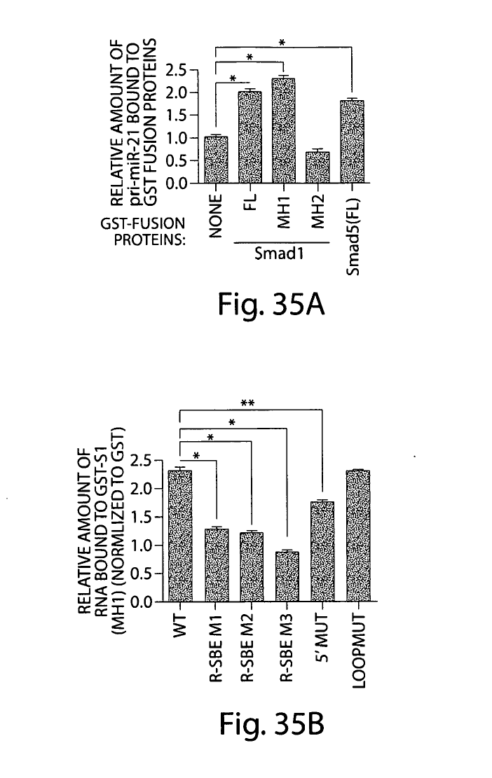

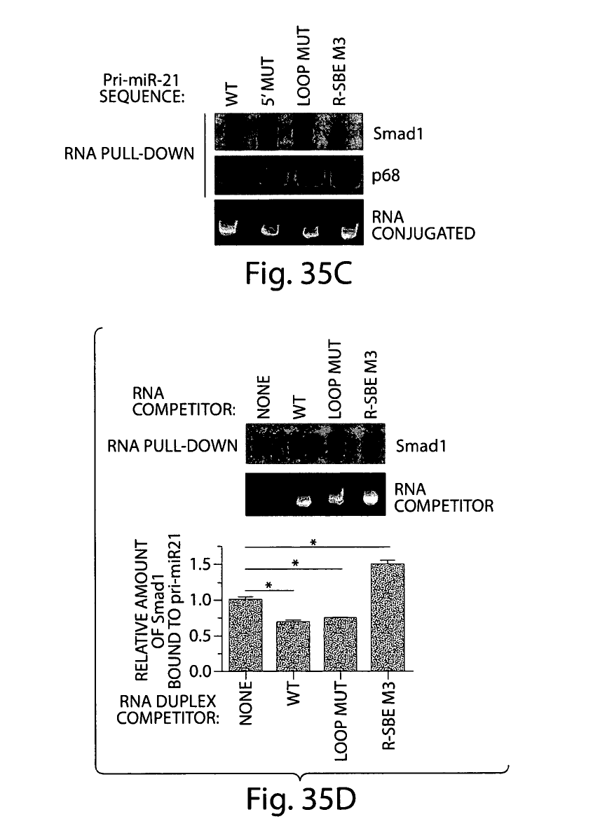

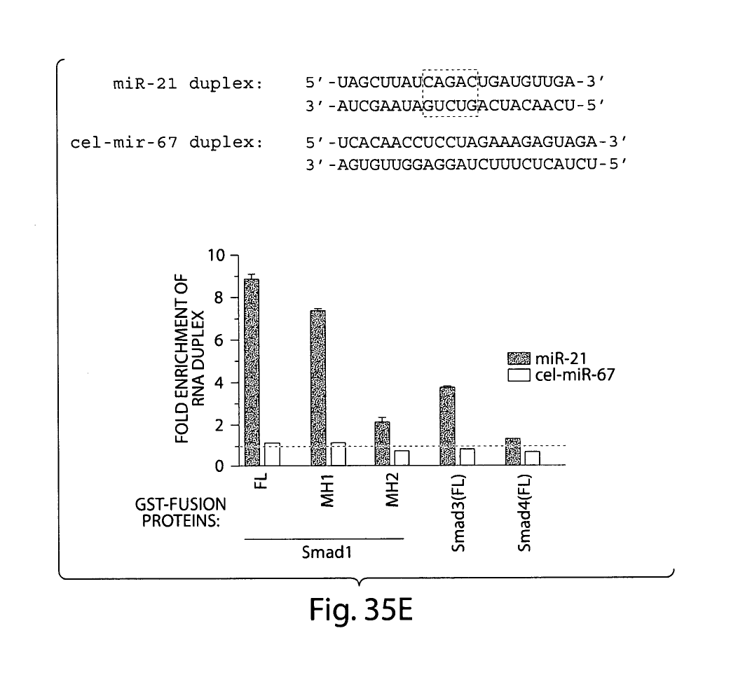

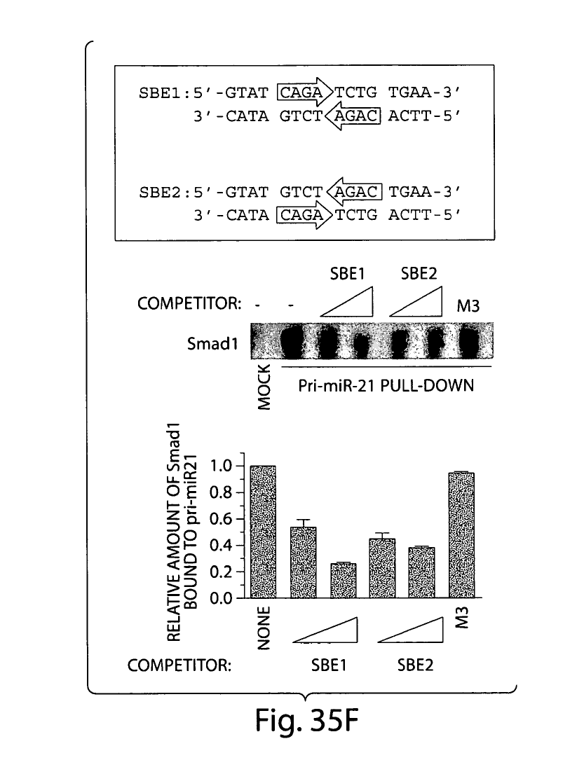

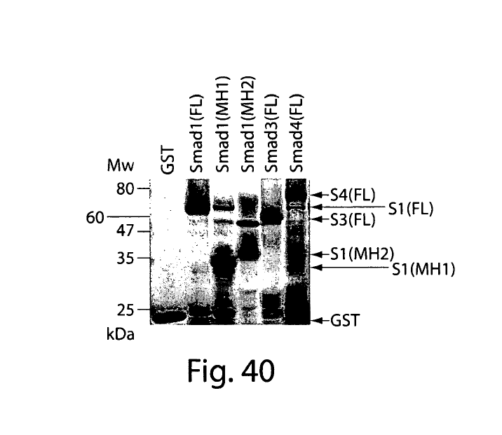

We have shown previously that the amino-terminal MH1 domain of Smad1, which contains the DNA binding domain, can associate with pri-miR-21 or pri-miR-199a upon BMP4 treatment in vivo (Davis et al., 2008). As Smad1(MH1) is unable to interact with p68 and be recruited by it to the pri-miRNA/Drosha complex, we speculated that Smad1 might directly contact the primary transcripts of the TGFβ-regulated miRNAs. To examine if R-Smads can directly interact with pri-miRNA and to map the region of R-Smads required for this association, we partially purified bacterially-expressed GST-Smad fusion proteins (FIG. 40), conjugated them to glutathione S-sepharose beads, and used them to pull down in vitro transcribed ˜150 nt pri-miR-21. The pri-miR-21 transcripts co-precipitating with GST-Smad fusion proteins were quantitated by qRT-PCR analysis (FIG. 35A). Full-length (FL) Smad1 and its MH1 domain were able to pull-down about two-fold more pri-miR-21 in comparison to GST protein alone (FIG. 35A). The carboxyl terminus (C-ter) MH2 domain of Smad1, which is required for interaction with p68 (Davis et al., 2008), did not bind pri-miR-21 (FIG. 35A). Full-length Smad5 and Smad1 interacted with pri-miR-21 at similar levels (FIG. 35A). Thus, the MH1 domain of R-Smads is sufficient to directly interact with pri-miR-21 in vitro. To determine the role of R-SBE in the association with R-Smad, in vitro transcribed pri-miR-21 transcripts, WT or mutants (shown in FIG. 34A), were pulled down with the GST-Smad1(MH1) fusion protein and quantitated by qRT-PCR analysis. All three R-SBE mutants (R-SBE M1-M3) showed a dramatic decrease in binding to Smad1(MH1) compared to WT pri-miR-21 (FIG. 35B). Conversely, the Loop and the 5′ mutants bound Smad1(MH1) with efficiency comparable to WT pri-miR-21 (FIG. 35B), indicating that the R-SBE is specifically required for association with R-Smad MH1, while the terminal loop and the stem sequence upstream of the R-SBE in pri-miR-21 are not critical for R-Smad binding.

The sequence-specific interaction between R-Smad and pri-miRNAs was further explored by using in vitro-generated pri-miR-21 transcripts, either wild type or mutants (5′ Loop or R-SBE M3), immobilized to agarose beads and incubated with nuclear extracts from cells treated with BMP4. The levels of Smad1 or p68 precipitated by the immobilized pri-miR-21 were examined by immunoblotting. WT, “5′ mut”, and “Loop mut” were able to precipitate Smad1, while the R-SBE M3 was not (FIG. 35C). p68 co-precipitated with all the pri-miR-21 constructs tested (FIG. 35C). Therefore, unlike Smad1, association of p68 with pri-miR-21 does not require the R-SBE (FIG. 35C). When an excess of in vitro transcribed pri-miR-21, WT or “Loop mut”, was added as a competitor to the immobilized pri-miR-21(WT), both WT and “Loop mut” competed equally with pri-miR-21 for Smad1 binding (FIG. 35D). R-SBE M3, however, did not compete for binding to Smad1 (FIG. 35D), confirming that the R-SBE is crucial for R-Smad binding. Again, the mutation of the terminal loop region had no consequence. However, the presence of the loop could still play a structural role in facilitating the interaction with R-Smads. To test this hypothesis, we examined the direct association of bacterially-expressed and partially purified GST-Smad (MH1) with a synthetic 22 by RNA duplex with sequence matching miR-21 (FIG. 35E, top panel). As a control, RNA duplex with C. elegans miR-67 sequence, which does not contain CAGAC sequence, was used (FIG. 35E, top panel). GST-fused Smad1(FL) and Smad3(FL) were found to specifically bind to the miR-21 duplex, while Smad4(FL) did not interact with either miR-21 or control duplexes (FIG. 35E). This is consistent with the previous observation that R-Smads but not Smad4 is essential for the regulation of pri-miRNA processing (Davis et al., 2008). Deletion of the MH1 domain of Smad1 exhibited a dramatic reduction in miR-21 binding in comparison with the full-length Smad1 (FIG. 35E, MH2). Consistently, the MH1 domain alone was sufficient for specific interaction with miR-21 duplex, similarly to Smad-DNA association (FIG. 35E, MH1). Altogether, these results confirm that the MH1 domain of R-Smads recognizes and binds the R-SBE in the context of a short dsRNA stem. Furthermore, as the interaction between bacterially expressed R-Smads and chemically synthesized RNA duplexes does not involve other eukaryotic proteins, it is consistent with the hypothesis that R-Smads bind the R-SBE directly.

### D-SBE Competes for Smad Binding to the R-SBE

A direct interaction between R-Smads and both dsRNA and DNA begs the question whether these two functions coexist or are mutually exclusive. We measured Smad1 binding in vitro to pri-miR-21 in the presence of excess competitors consisting of 16 by DNA oligonucleotides with SBE sequence 1 or 2 (FIG. 35F, top panel). SBE1 DNA contains two copies of the R-SBE sequence in palindrome (5′-CAGATCTG-3′) and SBE2 DNA contains two copies of the D-SBE in palindrome (5′-GTCTAGAC-3′), which has been shown to bind to two molecules of the MH1 domain of Smad in vitro (Shi et al., 1998). As a negative control, we used the in vitro transcribed pri-miR-21 mutant M3, which binds only weakly to Smad1 (see FIG. 35B). Both DNA duplexes similarly competed for Smad1 binding to pri-miR-21 (FIG. 35F, middle and bottom panel). Thus, our results are consistent with a model whereby binding of Smads to D-SBE and R-SBE is mediated through the same region of the MH1 domain and is mutually exclusive.

Introduction of an R-SBE is Sufficient to Enable BMP/TGFβ-Mediated Regulation of Processing of pri-miRNAs

Finally, we examined whether introduction of an R-SBE sequence is sufficient to confer TGFβ-mediated regulation at the pri- to pre-miRNA processing step to an otherwise unregulated pri-miRNA. A portion of approximately 150 bp of the C. elegans pri-miR-84 sequence (cel-miR-84) was placed under control of a constitutive CMV promoter, and an R-SBE was inserted in three alternative locations of the stem region of pre-miR-84 by nucleotide substitutions (FIG. 36A, left panel). Each cel-miR-84 expression construct was transfected in 10T1/2 cells and BMP-dependent processing of these constructs was examined (FIG. 36A, right panel). Neither transcription nor processing of the wild type cel-miR-84 pri-miRNA was regulated by BMP4, as predicted due to lack of an R-SBE sequence in cel-miR-84 (FIG. 36A, WT). Introduction of the R-SBE to pri-cel-miR-84 did not affect the level of pri-miRNA after BMP4 treatment (FIG. 36A, M1-M3, Pri-miRNA). When the R-SBE sequence was introduced in the middle of the mature cel-miR-84 sequence (M2), the processing of pri-cel-miR-84 became inducible by BMP4 and pre-cel-miR-84 expression was increased 2.4-fold upon BMP4 treatment, similarly to pre-miR-21 (2.5-fold induction) (FIG. 36A, M2 and miR-21, Pre-miRNA), indicating that presence of R-SBE in the mature miRNA sequence is sufficient to bestow responsiveness to BMP treatment in a pri-miRNA. Interestingly, introduction of the R-SBE in the stem region of pri-cel-miR-84 either upstream (M1) or downstream (M3) of the mature miR-84 sequence had no effect on the processing, indicating that the position of the R-SBE within the stem region of the pre-miRNA plays a critical role (FIG. 36A, M1 and M3, Pre-miRNA). Altogether, these results confirm that association of R-Smads with an R-SBE located within the stem region of pri-miRNAs encoding the mature miRNA sequence facilitates miRNA processing in a TGFβ/BMP-dependent fashion (FIG. 36B).

### SUMMARY

We demonstrated that direct association of Smad proteins with a D-SBE-like sequence in mature miRNAs operates as a molecular tag for Drosha and DGCR8 recognition and preferential association with a set of pri-miRNAs, facilitating their processing by Drosha upon TGFβ or BMP4 stimulation (FIG. 36B).

### R-Smads Bind Both DNA and RNA

We demonstrated that Smad proteins bind to dsRNA containing the 5′-CAGAC-3′ sequence. Although the optimal DNA binding site for Smads was initially identified as an 8 by palindromic motif (5′-GTCTAGAC-3′), the crystal structure of the Smad3 MH1 domain bound to DNA with this palindromic SBE revealed that two MH1 molecules bind independently to the major groove of each half-motif (5′-GTCT-3′) (Chai et al., 2003). Indeed, a 4 by half-motif can be found in many promoters of TGFβ/BMP target genes, such as Id3, Xvent-2, and PAI-1 (Massague and Wotton, 2000). The structural analysis also indicates that there is no direct contact between amino acids in the MH1 domain of Smad and the two thymine residues in the GTCT sequence (Chai et al., 2003). It is interesting that R-Smads recognize the same sequence in their interaction with double stranded RNA and DNA. Although the exact structure of Smads binding to R-SBE needs to be resolved, it is believed that the potential imperfect A-form RNA duplex structure of pri-miRNAs may allow it to occupy the same protein cavity of Smad proteins that receives the B-form DNA duplex.

Smads are not the only proteins able to bind both forms of nucleic acids. p53, a DNA binding protein which also binds RNA, inhibits translation of Cyclin dependent kinase 4 (Cdk4); this effect is dependent on the 5′-UTR of the Cdk4 mRNA, but does not require direct binding of p53 to mRNA (Miller et al., 2000). Although there is no direct evidence that p53 regulates translation of Cdk4 mRNA via miRNA, it is intriguing to speculate that p53 and Smads, both of which have DNA and RNA binding ability, play dual functions in transcription as well as miRNA biogenesis. Inducible association of transcription factors with pri-miRNA sequences could be a broad mechanism of rapid miRNA processing regulation. In addition to Smad and p53, other transcription factors known to bind both DNA and RNA are TFIIIA, Stat1 and WT1 (Cassiday and Maher, 2002). It was also reported that NFκB binds sequence-specifically to a DNA duplex and to a synthetic RNA aptamer predicted to form a stem-bulge-stem-loop structure with indistinguishable affinity and stoichiometry (Cassiday et al., 2002).

### Variants of the R-SBE Sequence

An analysis of miRNA sequences encoded in the human genome revealed that approximately 5% (36 out of 706) of the miRNAs contain an R-SBE (5′-CAGAB-3′, see Table 2). While the majority of R-SBE sequences analyzed (5/7) occur on the 5′ arm of the double-stranded stem, miR-421 and miR-600 are encoded on the 3′ arm, suggesting that Smad-mediated processing is independent of the strand within the hairpin. All the R-SBE miRNAs analyzed containing a cytosine in 5^{th} position (5′-CAGAC-3′ sequence), with a notable exception of miR-214 (see below), were induced by TGF or BMP4 in PASMC (Table 2). Thus, our results indicate that an R-SBE in the mature miRNA sequence is a molecular signature for the miRNAs whose biosynthesis is controlled by the TGFβ-Smad signaling pathway. Interestingly, we have found that miR-23a and -23b, which contain a 5′-CAGGG-3′ sequence, are also regulated post-transcriptionally by TGFβ and BMP4 through Smads at the first processing step (FIG. 31A and data not shown). Of the miRNAs detected in the miRNA microarray and containing a 5′-CAGGG-3′ sequence, 9 out of 10 are induced by TGFβ and/or BMP4 (Table 2), indicating that 5′-CAGGG-3′ serves as a Smad binding sequence. This finding is also consistent with reports that Smads can bind to G/C-rich sequences in DNA (Ishida et al., 2000). Nearly all nucleic acids residues in the R-SBE form base pairing in the stem region of pre-miRNAs (Table 2). Interestingly, the second adenine residue of miR-21 does not form a base pairing and generates a single nucleotide “bulge”. In the case of miR-214, which contains a 5′-CAGAC-3′ sequence but is not induced by TGFβ or BMP4, the first cytosine residue of the R-SBE is part of a 3 nt single-stranded “bubble” region. Therefore, we speculate that R-Smads might be able to associate with R-SBE containing a single nucleotide bulge but not with an R-SBE in a bubble region.

### Requirement of Smad4

Similarly to our findings with miR-21 and miR-199a (Davis et al., 2008), Smad4 is not required for regulation of Drosha processing in the newly identified TGFβ/BMP-regulated miRNAs. It is of note that the degree of induction of pre-miRNA by TGFβ/BMP is larger in Smad4-null cells, such as MDA-MB468 cells, in comparison with Smad4-expressing PASMCs or Cos7 cells, suggesting that Smad4 might be inhibitory to the regulation of miRNA biosythesis by R-Smads (see FIG. 32B). Congruently, knock down of Smad4 in PASMCs slightly elevated pre-miR-21 induction by BMP4 (Davis et al., 2008). It was previously reported that R-Smads and Smad4 translocate to the nucleus as a complex. A more recent study, however, demonstrates that R-Smads and Smad4 can be independently transported into the nucleus through different nuclear import machineries (Yao et al., 2008). Thus, we speculate that R-Smads that are not locked into a complex with Smad4 might preferentially associate with R-SBE and participate in the Drosha/DGCR8 complex. In contrast, the R-Smad/Smad4 heteromeric complex might preferentially associate with D-SBE and act as a transcription factor. Unlike the association of Smads with R-SBE, the MH1 domains of both R-Smads and Smad4 bind to D-SBE with similar affinity (Shi et al., 1998). It is currently unclear why Smad4 exhibits less affinity for the R-SBE in comparison to R-Smads. It was previously reported that the intramolecular interaction between the MH1 and the MH2 domains of Smad4 masks the DNA binding activity of the MH1 domain when Smad4 is not forming a complex with R-Smads (Lagna et al., 1996); our results indicate that a similar mechanism might be preventing Smad4 from binding to the R-SBE.

### Recruitment of Drosha and DGCR8 to R-SBE-Bound Smads

We showed that association of Smads with R-SBE is important for the ligand-induced recruitment of Drosha and DGCR8. We found that a GST-Smad1 fusion protein is able to interact with DGCR8 under conditions that do not allow Drosha interaction (B. D. and A. H., unpublished observation), indicating a potential direct contact between R-Smads and DGCR8. Thus, our results indicate that when a Smad binds to pri-miRNAs, it provides an optimal landing site for DGCR8 and (possibly indirectly) for Drosha, and thereby promotes more efficient cleavage of specific pri-miRNAs. Smad binding to the R-SBE may induce an alteration in the pri-miRNA structure that is more favorably recognized and bound by Drosha and DGCR8. Alternatively, Smad may recruit to the R-SBE auxiliary factors, such as p68, which then facilitate the recruitment of Drosha/DGCR8 to specific pri-miRNAs. Unlike DGCR8, which contains two dsRNA-binding domains (dsRBD) and binds directly to pri-miRNAs, Drosha has one dsRBD and binds weakly to pri-miRNAs (Kim et al.). The observation that the in vitro pri-miRNA processing reaction with purified Drosha is inefficient and inaccurate also points to the requirement of accessory factor(s) for the efficient cleavage of pri-miRNA by Drosha (Kim et al.). Our results also indicate that Smad proteins may stabilize the association of Drosha with pri-miRNA through direct association with pri-miRNA, similarly to a function ascribed to DGCR8 (Han et al., 2006; Han et al., 2009). We reported that the MH2 domain of R-Smads interacts with the p68 RNA helicase. As p68 contains an independent RNA binding ability, p68 may have a role as a RNA binding cofactor for Smads and thus facilitates Smad-R-SBE association.

### Role of Smad-R-SBE Association and the Mechanism of Regulation

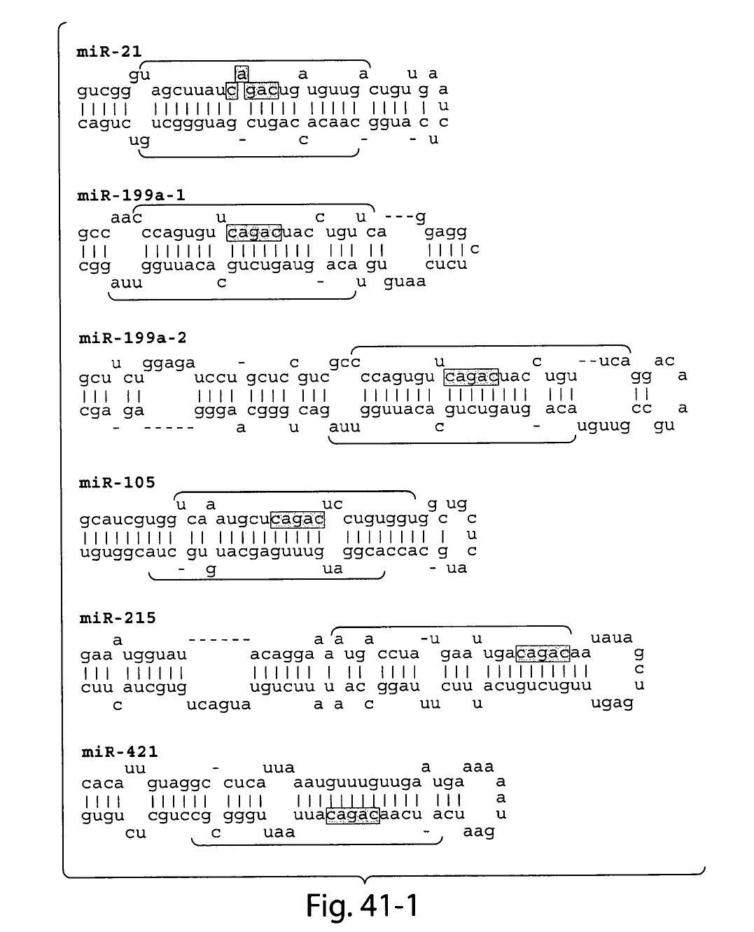

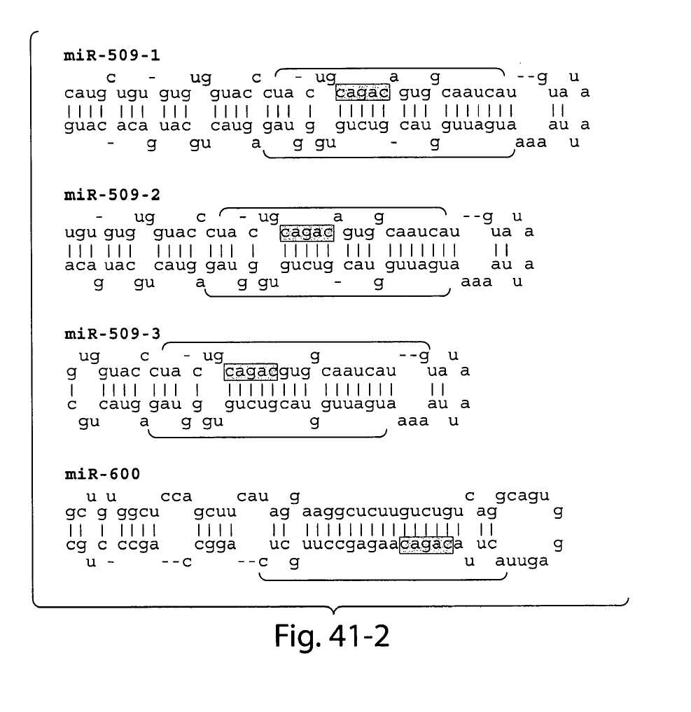

A typical metazoan pri-miRNA consists of a 33-bp stem in which mature miRNA is encoded ˜11 by from the dsRNA-ssRNA junction, as well as the terminal loop and ssRNA flanking sequence (see FIG. 36B). Recently, it was demonstrated that DGCR8 associates with pri-miRNAs and serves as a molecular ruler to measure the distance from the dsRNA-ssRNA junction where it positions Drosha (Han et al., 2006). We found that the R-SBE is located within the mature miRNA; 4-12 bp away from the Drosha cleavage site, and ˜9 bp away from the 5′-end (+10) of the mature miRNA (FIG. 36B). A study of chimeric pri-cel-miR-84 in which the R-SBE sequence was introduced in the different regions of the stem indicates that the position of the R-SBE within the mature miRNA is critical for the TGFβ-mediated regulation of processing (FIG. 36A). Interestingly, the R-SBE site does not overlap with the seed sequence (+2 to +8), which is critical for recognition and association of miRNA with target mRNAs (FIG. 41). Therefore, R-SBE is not directly involved in the recognition of target mRNAs (see FIG. 41). Thus, we hypothesize that a role for the Smad-R-SBE interaction might be the correct positioning of the Drosha/DGCR8 complex on the stem-loop structure. Therefore, this role of Smads in Drosha processing would be analogous to their role in transcriptional regulation, where they position RNA Pol II at the transcription initiation site. Interestingly, the R-SBE is located on average 10 by from the terminal loop overlapping with an abortive processing site which is ˜11 by from the terminal loop. Association of Smad with R-SBE, therefore, might mask the abortive processing reaction and facilitate a productive processing whose product contains a full miRNA sequence.

### Other Drosha-Interacting Proteins

Similar to our study on the regulation of Drosha processing by Smad proteins, it has been reported that hnRNP A1 post-transcriptionally facilitates the processing of pri-miR-18a by Drosha. hnRNP A1, a nucleo-cytoplasmic shuttling protein, belongs to a large family of RNA binding proteins that are components of messenger ribonucleoprotein complexes (mRNPs) and are involved in many aspects of mRNA metabolism, including precursor mRNA splicing. It was shown that hnRNP A1 binds directly to the terminal loop region of pre-miR-18a and other pre-miRNAs that contain the hnRNP A1 binding sequence, and promotes miRNA cropping by Drosha, presumably through structural rearrangement of the RNA stem caused by binding of hnRNP A1 to the terminal loop (Guil and Caceres, 2007; Michlewski et al., 2008). A change of RNA conformation induced by protein binding has been observed also in other systems. For example, proteins encoded by the human immunodeficiency virus (HIV), such as Tat and Rev, bind to the major groove of A-form RNA and increase the major groove widths of target RNAs (Battiste et al., 1996; Puglisi et al., 1993). On the other hand, the crystal structure of the Smad-MH1 domain in complex with DNA indicates that Smad binding alters the local conformation of DNA (Shi et al., 1998). Thus, Smads may also alter the local conformation of the stem region of pri-miRNAs upon binding to the R-SBE, thereby facilitating recruitment and association of Drosha, DGCR8 and possibly other auxiliary factors. Recently, the subcellular localization of hnRNP A1 has been shown to be regulated through phosphorylation by the p38 mitogen-activated protein kinase (MAPK)(Shimada et al., 2009). Nucleo-cytoplasmic transport of Smad proteins is tightly controlled by phosphorylation of serine residues at the C-terminus, which is mediated by the TGFβ type I receptor kinases. Interestingly, MAPK and glycogen synthase kinase 3 (GSK3) can also alter Smad subcellular localization through phosphorylation in the linker region (Fuentealba et al., 2007; Kretzschmar et al., 1997). Therefore, Smad-dependent regulation of miRNA biosynthesis could be modulated independently of TGFβ and BMPs by signals that alter the nuclear localization of Smads, such as the ERK-MAPK and the Wnt pathways.

Smad nuclear interacting protein 1 (SNIP1) was originally identified as a nuclear partner of Smad proteins (Kim et al., 2000) and shown to modulate transcription of the Cyclin D1 gene (Roche et al., 2004). SNIP1 has recently been shown to regulate the stability of Cyclin D1 mRNA by recruiting the RNA processing factor U2AF64 to the 3′-UTR of Cyclin D1 mRNA (Bracken et al., 2008), suggesting that SNIP1, similarly to Smads, is able to modulate gene expression through two distinct mechanisms: regulation of transcription and mRNA stability. More recently, both the Arabidopsis ortholog of SNIP1, DAWDLE (DDL), and human SNIP1 were found to interact with Drosha (DCL1 in Arabidopsis) and modulate miRNA biogenesis (Goertzel et al., 2008). Therefore, it is possible that SNIP1, in complex with Smad proteins, participates in the regulation of the processing of pri-miRNAs by Drosha, as well as transcriptional regulation.

Without wishing to be bound by any particular theories disclosed herein, this work provides a molecular basis for the specific regulation of a set of miRNAs by the TGFβ signaling pathway and a role of Smad proteins as accessory factors for the Drosha/DGCR8 microprocessor. Phylogenetically conserved sequences in the stems or terminal loops of pri-miRNAs, especially when not included in the miRNA seed sequence, effect a regulatory function, likely as platforms for the recruitment of accessory factors, such as Smads and hnRNP A1, which then promote efficient processing by Drosha. It is believed that a conserved sequence might also serve as a mechanism to coordinate expression of a group of miRNAs in response to a growth factor signal or other physiological stimulus.

### Experimental Procedures

Cell culture. Human primary pulmonary artery smooth muscle cells (PASMCs) were purchased from Lonza (#CC-2581) and maintained in Sm-GM2 media (Lonza) containing 5% fetal bovine serum (FBS, Sigma). Cos7, MDA-MB468 and C3H10T1/2 cells (American Type Culture Correction) were maintained in Dulbecco's Modified Eagle media (DMEM) supplemented with 10% FBS (Sigma).

Growth factor stimulation and plasmid transfection: Recombinant human TGFβ1 (#240-B-002) and BMP4 (#314-BP-010) were purchased from R&D Systems. All growth factor stimulations were performed under starvation conditions (0.2% FBS). All plasmid transfections were performed using Fugene6 (Roche).

RNA Interference. Synthetic small interference RNA (siRNA) targeting human Smad1 or Smad5 were obtained from Invitrogen (Validated Stealt™ DuoPak) and transfected into PASMC using RNAimax (Invitrogen). A siRNA with a non-targeting sequence (scramble siRNA, Dharmacon) was used as a negative control.

Antibodies. Anti-Flag epitope tag (M2, Sigma), anti-p68 (clone PAb204, Upstate), anti-GAPDH (2E3-2E10, Abnova), anti-Smad1/Smad5/Smad8 (Calbiochem), anti-DGCR8 (#10996-1-AP, ProteinTech Group) and anti-Drosha (#07-717, Upstate) antibodies. Protein quantiation was preformed by densitometry using ImageJ gel analysis software (rsbweb.nih.gov/ij/).

Immunoblot assay. Cells were lysed in THE buffer (1% Nonidet P-40, 10 mM Tris-HCl (pH 7.5), 1 mM EDTA, 150 mM NaCl). Total cell lysates or affinity purified proteins were separated on a SDS-PAGE, transferred to PVDF membranes (Millipore), immunoblotted with antibodies, and visualized using an enhanced chemiluminescence detection system (Amersham Biosciences).

MiRNA microarray: Applied Biosystems microRNA array A v2.0 was used to quantitate microRNA levels of 377 human microRNAs according to the manufacturer's directions. Total RNA (850 ng) was isolated from PASMC treated with vehicle, recombinant human BMP4 or TGFβ1 R&D Systems) for 24 hr. Processing and k-means cluster analysis of microRNAs altered at least +/−1.6 fold by growth factor treatment was performed by GenePattern (Reich et al., 2006). Heat map was generated by Java Treeview (Saldanha, 2004).

Identification of conserved sequence. Sequence motif detection was performed by Improbizer program (on the web at: cse.ucsc.edu/˜kent/improbizer/improbizer.html) on cluster 1, including miR-21, -23b, -105, -199b, -215, -421, -509, -127, -107, -508-3p, -542-3p, -522, -409-5p, -200c, -489, -101, -455-3p, -362-3p, let-7b, 502-3p, -486-3p. Conserved sequence was aligned and sequence logo was generated using WebLogo (Crooks et al., 2004).

Real-time RT-PCR analysis. Real-time RT-PCR analysis was performed as previously described (Davis et al., 2008).

RNA-Immunoprecipitation (RNA-IP) assay. RNA-IP was performed as previously described (Davis et al., 2008).

Synthetic RNA/DNA duplex. DNA or RNA duplexes were synthesized by IDT. Linear range of detection by Taqman miRNA qRT-PCR was obtained with 10⁻¹²-10⁻¹⁶ M synthesized dsRNA in a complex mixture of tRNA. RNA was heated to 65° C. for 5 min and quickly placed on ice prior to reverse transcription to ensure melting of the double strand.

In vitro RNA synthesis. Pri-miR-21 wildtype and mutants were in vitro transcribed from the T7 promoter of pcDNA3.1(+).

In vitro GST-SMAD-RNA pull down assay. In-vitro interaction of GST-fusion proteins and RNA was performed essentially as previously described (Davis et al., 2008).

RNA affinity purification. In vitro transcribed RNAs were covalently linked to adipic acid dihydrazide agarose beads as previously described (Guil and Caceres, 2007).

Statistical Analysis. The results presented are average of at least three experiments each performed in triplicate with standard errors. Statistical analyses were performed by analysis of variance, followed by Tukey's multiple comparison test or by Student's t test as appropriate, using Prism 4 (GraphPAD Software Inc.). P values of <0.05 were considered significant and are indicated with asterisks.

### REFERENCES

Bartel, D. P. (2004). MicroRNAs: genomics, biogenesis, mechanism, and function. Cell 116, 281-297.
Battiste, J. L., Mao, H., Rao, N. S., Tan, R., Muhandiram, D. R., Kay, L. E., Frankel, A. D., and Williamson, J. R. (1996). Alpha helix-RNA major groove recognition in an HIV-1 rev peptide-RRE RNA complex. Science 273, 1547-1551.
Berezikov, E., and Plasterk, R. H. (2005). Camels and zebrafish, viruses and cancer: a microRNA update. Hum Mol Genet. 14 Spec No. 2, R183-190.
Bracken, C. P., Wall, S. J., Barre, B., Panov, K. I., Ajuh, P. M., and Perkins, N. D. (2008). Regulation of cyclin D1 RNA stability by SNIP1. Cancer Res 68, 7621-7628.
Cahn, G. A., and Croce, C. M. (2006). MicroRNA signatures in human cancers. Nat Rev Cancer 6, 857-866.
Carthew, R. W., and Sontheimer, E. J. (2009). Origins and Mechanisms of miRNAs and siRNAs. Cell 136, 642-655.
Cassiday, L. A., Lebruska, L. L., Benson, L. M., Naylor, S., Owen, W. G., and Maher, L. J., 3rd (2002). Binding stoichiometry of an RNA aptamer and its transcription factor target. Anal Biochem 306, 290-297.
Cassiday, L. A., and Maher, L. J., 3rd (2002). Having it both ways: transcription factors that bind DNA and RNA. Nucleic Acids Res 30, 4118-4126.
Chai, J., Wu, J. W., Yan, N., Massague, J., Pavietich, N. P., and Shi, Y. (2003). Features of a Smad3 MH1-DNA complex. Roles of water and zinc in DNA binding. J Biol Chem 278, 20327-20331.
Coller, H. A., Forman, J. J., and Legesse-Miller, A. (2007). “Myc'ed messages”: myc induces transcription of E2F1 while inhibiting its translation via a microRNA polycistron. PLoS Genet. 3, e146.
Crooks, G. E., Hon, G., Chandonia, J. M., and Brenner, S. E. (2004). WebLogo: a sequence logo generator. Genome Res 14, 1188-1190.
Cullen, B. R. (2006). Viruses and microRNAs. Nat Genet. 38 Suppl, S25-30.
Davis, B. N., Hilyard, A. C., Lagna, G., and Hata, A. (2008). SMAD proteins control DROSHAmediated microRNA maturation. Nature 454, 56-61.
Dennler, S., Itoh, S., Vivien, D., ten Dijke, P., Huet, S., and Gauthier, J. M. (1998). Direct binding of Smad3 and Smad4 to critical TGFβ-inducible elements in the promoter of human plasminogen activator inhibitor-typel gene. Embo J. 17, 3091-3100.
Fuentealba, L. C., Eivers, E., Ikeda, A., Hurtado, C., Kuroda, H., Pera, E. M., and De Robertis, E. M. (2007). Integrating patterning signals: Wnt/GSK3 regulates the duration of the BMP/Smad1 signal. Cell 131, 980-993.
Fukao, T., Fukuda, Y., Kiga, K., Sharif, J., Hino, K., Enomoto, Y., Kawamura, A., Nakamura, K., Takeuchi, T., and Tanabe, M. (2007). An evolutionarily conserved mechanism for microRNA-223 expression revealed by microRNA gene profiling. Cell 129, 617-631.
Fukuda, T., Yamagata, K., Fujiyama, S., Matsumoto, T., Koshida, I., Yoshimura, K., Mihara, M., Naitou, M., Endoh, H., Nakamura, T., et al. (2007). DEAD-box RNA helicase subunits of the Drosha complex are required for processing of rRNA and a subset of microRNAs. Nat Cell Biol 9, 604-611.
Goertzel, B., Pennachin, C., de Alvarenga Mudado, M., and de Souza Coelho, L. (2008). Identifying the genes and genetic interrelationships underlying the impact of calorie restriction on maximum lifespan: an artificial intelligence-based approach. Rejuvenation Res 11, 735-748.
Guil, S., and Caceres, J. F. (2007). The multifunctional RNA-binding protein hnRNP A1 is required for processing of miR-18a. Nat Struct Mol Biol 14, 591-596.
Hammond, S. M. (2006). RNAi, microRNAs, and human disease. Cancer Chemother Pharmacol 58 63-68.
Han, J., Lee, Y., Yeom, K. H., Nam, J. W., Heo, I., Rhee, J. K., Sohn, S. Y., Cho, Y., Zhang, B. T., and Kim, V. N. (2006). Molecular basis for the recognition of primary microRNAs by the Drosha-DGCR8 complex. Cell 125, 887-901.
Han, J., Pedersen, J. S., Kwon, S. C., Belair, C. D., Kim, Y. K., Yeom, K. H., Yang, W. Y., Haussler, D., Blelloch, R., and Kim, V. N. (2009). Posttranscriptional crossregulation between Drosha and DGCR8. Cell 136, 75-84.
Heo, I., Joo, C., Cho, J., Ha, M., Han, J., and Kim, V. N. (2008). Lin28 mediates the terminal uridylation of let-7 precursor MicroRNA. Mol Cell 32, 276-284.
Ishida, W., Hamamoto, T., Kusanagi, K., Yagi, K., Kawabata, M., Takehara, K., Sampath, T. K., Kato, M., and Miyazono, K. (2000). Smad6 is a Smad1/5-induced smad inhibitor. Characterization of bone morphogenetic protein-responsive element in the mouse Smad6 promoter. J Biol Chem 275, 6075-6079.
Kim, R. H., Flanders, K. C., Birkey Reffey, S., Anderson, L. A., Duckett, C. S., Perkins, N. D., and Roberts, A. B. (2001). SNIP1 inhibits NF-kappa B signaling by competing for its binding to the C/H1 domain of CBP/p300 transcriptional co-activators. J Biol Chem 276, 46297-46304.
Kim, R. H., Wang, D., Tsang, M., Martin, J., Huff, C., de Caestecker, M. P., Parks, W. T., Meng, X., Lechleider, R. J., Wang, T., and Roberts, A. B. (2000). A novel smad nuclear interacting protein, SNIP1, suppresses p300-dependent TGF-beta signal transduction. Genes Dev 14, 1605-1616.
Kim, V. N. (2005). MicroRNA biogenesis: coordinated cropping and dicing. Nat Rev Mol Cell Biol 6, 376-385.
Kim, V. N., Han, J., and Siomi, M. C. (2009). Biogenesis of small RNAs in animals. Nat Rev Mol Cell Biol 10, 126-139.
Kim, V. N., and Nam, J. W. (2006). Genomics of microRNA. Trends Genet. 22, 165-173.
Korchynskyi, O., and ten Dijke, P. (2002). Identification and functional characterization of distinct critically important bone morphogenetic protein-specific response elements in the Id1 promoter. J Biol Chem 277, 4883-4891.
Kretzschmar, M., Doody, J., and Massague, J. (1997). Opposing BMP and EGF signalling pathway converge on the TGFβ family mediator Smad1. Nature 389, 618-622.
Lagna, G., Hata, A., Hemmati-Brivanlou, A., and Massague, J. (1996). Partnership between DPC4 and SMAD proteins in TGF-beta signalling pathways. Nature 383, 832-836.
Mallory, A. C., and Vaucheret, H. (2006). Functions of microRNAs and related small RNAs in plants. Nat Genet 38 Suppl, S31-36.
Massague, J., Seoane, J., and Wotton, D. (2005). Smad transcription factors. Genes Dev 19, 2783-2810.
Massague, J., and Wotton, D. (2000). Transcriptional control by the TGFβ/Smad signaling system. EMBO J. 19, 1745-1759.
Michael, M. Z., S M, O. C., van Hoist Pellekaan, N. G., Young, G. P., and James, R. J. (2003). Reduced accumulation of specific microRNAs in colorectal neoplasia. Mol Cancer Res 1, 882-891.
Michlewski, G., Guil, S., Semple, C. A., and Caceres, J. F. (2008). Posttranscriptional regulation of miRNAs harboring conserved terminal loops. Mol Cell 32, 383-393.
Miller, S. J., Suthiphongchai, T., Zambetti, G. P., and Ewen, M. E. (2000). p53 binds selectively to the 5′ untranslated region of cdk4, an RNA element necessary and sufficient for transforming growth factor beta- and p53-mediated translational inhibition of cdk4. Mol Cell Biol 20, 8420-8431.
Morlando, M., Ballarino, M., Gromak, N., Pagano, F., Bozzoni, I., and Proudfoot, N. J. (2008). Primary microRNA transcripts are processed co-transcriptionally. Nat Struct Mol Biol 15, 902-909.
Niwa, R., and Slack, F. J. (2007). The evolution of animal microRNA function. Curr Opin Genet Dev 17, 145-150.
Ozsolak, F., Poling, L. L., Wang, Z., Liu, H., Liu, X. S., Roeder, R. G., Zhang, X., Song, J. S., and Fisher, D. E. (2008). Chromatin structure analyses identify miRNA promoters. Genes Dev 22, 3172-3183.
Puglisi, J. D., Chen, L., Frankel, A. D., and Williamson, J. R. (1993). Role of RNA structure in arginine recognition of TAR RNA. Proc Natl Acad Sci USA 90, 3680-3684.
Reich, M., Liefeld, T., Gould, J., Lerner, J., Tamayo, P., and Mesirov, J. P. (2006). GenePattern 2.0. Nat Genet. 38, 500-501.
Roche, K. C., Wiechens, N., Owen-Hughes, T., and Perkins, N. D. (2004). The FHA domain protein SNIP 1 is a regulator of the cell cycle and cyclin D1 expression. Oncogene 23, 8185-8195.
Rybak, A., Fuchs, H., Smirnova, L., Brandt, C., Pohl, E. E., Nitsch, R., and Wulczyn, F. G. (2008). A feedback loop comprising lin-28 and let-7 controls pre-let-7 maturation during neural stem-cell commitment. Nat Cell Biol 10, 987-993.
Sakamoto, H., Iwasaki, S., Kushima, M., Shichijo, T., and Ogawa, Y. (2008). Traumatic bilateral testicular dislocation: a recovery of spermatogenesis by orchiopexy 15 years after the onset. Fertil Steril 90, 2009 e2009-2011.
Saldanha, A. J. (2004). Java Treeview—extensible visualization of microarray data. Bioinformatics 20, 3246-3248.
Shi, Y., Wang, Y.-F., Jayaraman, L., Yang, H., Massague, J., and Pavletich, N. (1998). Crystal structure of a Smad MH1 domain bound to DNA: Insights on DNA-binding in TGF-β signaling. Cell 94, 585-594.
Shimada, N., Rios, I., Moran, H., Sayers, B., and Hubbard, K. (2009). p38 MAP kinase-dependent regulation of the expression level and subcellular distribution of heterogeneous nuclear ribonucleoprotein A1 and its involvement in cellular senescence in normal human fibroblasts. RNA Biol 6.
Siomi, H., and Siomi, M. C. (2009). On the road to reading the RNA-interference code. Nature 457, 396-404.
Taganov, K. D., Boldin, M. P., and Baltimore, D. (2007). MicroRNAs and immunity: tiny players in a big field. Immunity 26, 133-137.
van Rooij, E., Liu, N., and Olson, E. N. (2008). MicroRNAs flex their muscles. Trends Genet. 24, 159-166.
Viswanathan, S. R., Daley, G. Q., and Gregory, R. I. (2008). Selective blockade of microRNA processing by Lin28. Science 320, 97-100.
Wiemer, E. A. (2007). The role of microRNAs in cancer: no small matter. Eur J Cancer 43, 1529-1544.
Wienholds, E., and Plasterk, R. H. (2005). MicroRNA function in animal development. FEBS Lett 579, 5911-5922.
Wulczyn, F. G., Smirnova, L., Rybak, A., Brandt, C., Kwidzinski, E., Ninnemann, O., Strehle, M., Seiler, A., Schumacher, S., and Nitsch, R. (2007). Post-transcriptional regulation of the let-7 microRNA during neural cell specification. Faseb J 21, 415-426.
Yao, X., Chen, X., Cottonham, C., and Xu, L. (2008). Preferential utilization of Imp7/8 in nuclear import of Smads. J Biol Chem 283, 22867-22874.

TABLE 2
List of CAGA or CAGG containing miRNAs in human genome

| CAGAC | CAGAA | CAGAU | CAGAG | CAGGG | CAGGG |
|---|---|---|---|---|---|
| hsa-miR-21*^{Ψ} | hsa-miR-148a* | hsa-miR-18a* | hsa-miR-127- | hsa-miR-23a* | hsa-miR-630 |
| | | | 5p* | | |
| hsa-miR- | hsa-miR-148b* | hsa-miR-106b* | hsa-miR-134* | hsa-miR- | hsa-miR-648 |
| 105*^{Ψ} | | | | 23b*^{Ψ} | |
| hsa- | hsa-miR-152* | hsa-miR-410* | hsa-miR-198** | hsa-miR-103* | hsa-miR-659 |
| miR-199a-5p* | | | | | |
| ^{Ψ} | | | | | |
| hsa-miR-214** | hsa-miR-195* | hsa-miR-542- | hsa-miR330- | hsa-miR-107* | hsa-miR-671- |
| | | 3p* | 3p** | | 3p** |
| hsa-miR- | hsa-miR-298 | hsa-miR-607 | hsa-miR-339- | hsa-miR-140- | hsa-miR-770- |
| 215*^{Ψ} | | | 3p* | 3p* | 5p |
| hsa-miR-300 | hsa-miR-342- | hsa-miR-1298 | hsa-miR-423- | hsa-miR-188- | hsa-miR-877 |
| | 3p* | | 5p* | 3p | |
| hsa-miR- | hsa-miR-361- | hsa-miR-1539 | hsa-miR-452* | hsa-miR-220c | hsa-miR-933 |
| 421*^{Ψ} | 5p* | | | | |
| hsa- | hsa-miR-378 | | hsa-miR-508- | hsa-miR-331- | hsa-miR-940 |
| miR-509-5p*^{Ψ} | | | 5p | 5p* | |
| hsa- | hsa-miR-383 | | hsa- | hsa-miR-345* | hsa-miR-1205 |
| miR-509-3-5p | | | miR-516a-3p | | |
| hsa-miR- | hsa-miR-422a* | | hsa-miR-518e* | hsa-miR-487a* | hsa- |
| 600*^{Ψ} | | | | | miR-1207-5p |
| hsa-miR-631* | hsa-miR-541 | | hsa- | hsa-miR-487b* | hsa-miR-1266 |
| | | | miR-520a-5p | | |
| hsa-miR-1208 | hsa-miR-567 | | hsa-miR-525- | hsa-miR-498 | hsa-miR-1290 |
| | | | 5p | | |
| hsa-miR-1284 | hsa-miR-654- | | hsa-miR-922 | hsa- | hsa-miR-1321 |
| | 5p* | | | miR-513a-5p | |
| hsa-miR-1292 | hsa-miR-921 | | hsa-miR-1912 | hsa-miR-612 | hsa-miR-1909 |
| hsa-miR-1324 | hsa-miR-1324 | | | hsa-miR-623 | hsa-miR-1915 |
| 87.5% | 100% | 100% | 75% | 90% | |

*miRNAs found to be induced by TGFβ and/or BMP4 in PASMCs.
**miRNAs not found to be regulated by TGFβ or BMP4 in PASMCs.
^{Ψ}miRNAs investigated in this study.
Other miRNAs are either not present on the miRNA array of this study or not expressed in PASMCs.
At the bottom of the table, percentages of miRNAs containing the indicated sequences at the top of the table which are regulated by either TGFβ or BMP4 in PASMCs

### Example 12

### Additional Materials and Methods

siRNA sequence: Smad1: 5;-GCAACCGAGUAACUGUGUCACCAUU-3 (SEQ ID NO: 42) Smad5: 5-CCUGAGUAUUGGUGUUCCAUUGCUU-3 (SEQ ID NO: 40)

MiRNA microarray: cDNA was generated by Megaplex reverse transcriptase reaction, added to Taqman universal PCR master mix, and applied to the array. Reactions were monitored using the Applied Biosystems 7900HT TLDA real time PCR system. Arrays were performed in duplicate and analyzed using comparative CT method in Applied Biosystems' RQ manager.

Human RT-PCR primers: GAPDH: 5′-ACCACAGTCCATGCCATCAC-3′ (SEQ ID NO: 24) and 5′-TCCACCACCCTGTTGCTGTA-3′ (SEQ ID NO: 25). Id3: 5′-ACTCAGCTTAGCCAGGTGGA-3′ (SEQ ID NO: 30) and 5′-AAGCTCCTTTTGTCGTTGGA-3′ (SEQ ID NO: 31). pri-miR-21: 5′-TGTTTTGCCTACCATCGTGA-3′ (SEQ ID NO: 84) and 5′-AAGTGCCACCAGACAGAAGG-3′ (SEQ ID NO: 85). pre-miR-21: 5′-TGTCGGGTAGCTTATCAGAC-3′ (SEQ ID NO: 22) and 5′-TGTCAGACAGCCCATCGAC-3′ (SEQ ID NO: 86). pri-miR-105-1: 5′-AAGTGCCACCAGACAGAAGG-3′(SEQ ID NO: 87) and 5′-AGAAACACAGAGCACAGGAA-3′ (SEQ ID NO: 88). pre-miR-105-1: 5′-TGTGCATCGTGGTCAAATGCT-3′ (SEQ ID NO: 89) and 5′-TAGACACCGTAGCACATGCTC-3′ (SEQ ID NO: 90). pri-miR-199a-1: 5′-GACCCCCAAAGAGTCAGACA-3′(SEQ ID NO: 91) and 5′-CTCTGAGCAGCCAAGGAAAC-3′ (SEQ ID NO: 92). pre-miR-199a-1:5′-GCCAACCCAGTGTTCAGACTA-3′ (SEQ ID NO: 49) and 5′-GCCTAACCAATGTGCAGACTA-3′ (SEQ ID NO: 50). pri-miR-215: 5′-ACTCTCATTTGATTCCAGCA-3′ (SEQ ID NO: 93) and 5′-CGTGGTGTTAGTCGATTTCT-3′ (SEQ ID NO: 94). pre-miR-215: 5′-ATCATTCAGAAATGGTATACA-3′ (SEQ ID NO: 95) and 5′-TTGAAGTAGCACAGTCATACA-3′ (SEQ ID NO: 96). pri-miR-421: 5′-ATCATTGTCCGTGTCTATGG-3′ (SEQ ID NO: 97) and 5′-CATTCTGAAGAGAGCTTGGA-3′ (SEQ ID NO: 98). pre-miR-421: 5′-GCACATTGTAGGCCTCATTAA-3′ (SEQ ID NO: 99) and 5′-GAGATCACAGAGCAGGCGCCC-3′ (SEQ ID NO: 100), pri-miR-509: 5′-miR-509-1: 5′-GCAGGAAACATAAGGAAAGA-3′ (SEQ ID NO: 101) and 5′-AGGGTAAAATACCTGCACTG-3′ (SEQ ID NO: 102). pre-miR-509-1: 5′-CATGCTGTGTGTGGTACCCTA-3′ (SEQ ID NO: 103) and 5′-CATGTGTCATGCAGTACTCTA-3′ (SEQ ID NO: 104). pre-miR-214: 5′-GGCCTGGCTGGACAGAGTTGT-3 (SEQ ID NO: 105) and 5′-AGGCTGGGTTGTCATGTGACT-3′ (SEQ ID NO: 106) pre-miR-222: 5′-GCTGCTGGAAGGTGTAGGTAC-3′ (SEQ ID NO: 107) and 5′-AGCTAGAAGATGCCATCAGAG-3′ (SEQ ID NO: 108). pre-miR-221: 5′-TGAACATCCAGGTCTGGGGCA-3′ (SEQ ID NO: 109) and 5′-GAGAACATGTTTCCAGGTAGC-3′ (SEQ ID NO: 110). TM49 (RNA-IP negative control): 5′-GCAAGCACATAGTGGAGCAA-3′(SEQ ID NO: 53) and 5′-TCAAACATCCAGGACAACCA-3′ (SEQ ID NO: 54).

RNA-Immunoprecipitation (RNA-IP) assay: PASMCs or Cos7 cells were crosslinked for 15 minutes with 1% formaldehyde and washed 2× with PBS. The cell pellet was then resuspended in Buffer A (5 mM PIPES, pH 8.0, 85 mM KCl, 0.5% Nonidet P-40). After 10 min on ice, the crude nuclei fraction was isolated, and then suspended in Buffer B (1% SDS, 10 mM EDTA, 50 mM Tris-HCl, pH 8.1). After fragmentation of chromatin by sonication, the lysates were subjected to immunoprecipitation o/n. Following immunoprecipitation with anti-Flag, anti-Smad1/5/8, Drosha or DGCR8 antibody, stringent washing, and elution, the RNA was isolated using Trizol-LS. Pellets were resuspended in 100 μl TE buffer and incubated with DNase I (10 U) for 30 min to remove any remaining DNA. Following extraction with phenol:chloroform (5:1), RNA was precipitated with ethanol and dissolved in 20 ul of water. 5 μl of RNA was used for a 20 μl cDNA synthesis reaction. qRT-PCR reactions were then performed using pre-miR primers.

In vitro GST-SMAD-RNA pull down assay: GST-smad fusion proteins were expressed in E. coli, isolated and bound to glutathione S-sepharose beads. Following partial purification, Smad bound beads were washed 4×5 min at 4° C. in wash buffer (10 mM Tris-HCl pH 7.6, 0.5M LiCl, 0.1% triton) and 1×10 at 4° C. in binding buffer (20 mM Tris-HCl pH 7.6, 0.1M KCl, 0.1% Tween-20, 0.1% Triton). Beads were resuspended in 100 ul binding buffer and pre-incubated with 25 ug tRNA, 1 ng poly-[dI-dC] and 2 μl RNAse inhibitor for 10 minutes before the addition of 10 pmoles synthesized mature miRNA or ˜20 pmoles in vitro transcribed 156 by pri-miR-21. Following 1 hr incubation with rocking at 4° C., beads were washed 4× with binding buffer. 2 consecutive elutions of bound RNA were performed by addition of 100 ul Elution buffer (1% SDS, 0.15M NaCl) followed by rocking at room temperature for 15 min. The eluates were combined, 600 ml Trizol-LS (Invitrogen) was added and the RNA was purified as usual. RNA was re-suspended in 20 μl water and 1 μl was used in a reverse transcriptase reaction followed by quantitative real time PCR to detect relative levels of RNA binding to Smad-GST fusion proteins.

Pri-miR-21 Expression construct: 156 by wild type pri-miR-21 sequence containing 42 by on both ends of the pre-miR-21 sequence was synthesized by PCR from pCMV-miR-21(Davis et al., 2008) using primers 21U: 5′-CCGGGATCCTGTTTGCCTACCATCGTGA-3′ (SEQ ID NO: 111) and 21D: 5′-CGGAATTCTGAGAACATTGGATATGGATGG-3′ (SEQ ID NO: 112). The primers were engineered to contain BamH1 (underlined in 21U) and EcoRI (underlined in 21D) restriction endonuclease sites to facilitate insertion into pcDNA3.1(+) vector. This wild type product was used for two step PCR mutagenesis to create the miR-21 mutants. In brief, the 21D and mutagenesis primers were used to synthesize the 3 portion of the insert, including the mutation site. This ˜70 by product was then used as a PCR primer with 21U to obtain a mutated, 156 pb product which was cloned into pcDNA3.1(+) using Barn H1 and Eco R¹ sites. Mutagenesis primers: R-SBE M1: 5′-TGTCGGGTAGCTTATAAAACTGATGTTGA-3′ (SEQ ID NO: 113), R-SBE M2: 5′-GGCAACACCTAACGATGGGCT-3′ (SEQ ID NO: 114), 5′ mut: 5′-CGGGTTAATTATCAGACTGAT-3′ (SEQ ID NO: 115), Loop mut: 5′-GACTGTTGACCATCATGGCAA-3′ (SEQ ID NO: 116). The R-SBE M3 mutant was generated using R-SBE M1 as the template and the R-SBEM2 primer. The sequence of all constructs was verified by DNA sequencing. Pri-miR-21 was detected using 21U and 21D, while pre-miRs were detected using human pre-miR-21 primers or corresponding mutagenesis primer.

C. elegans pri-miR-84 Expression construct: 152 by wild type or mutant pri-cel-miR-84 containing 38 bp flanking the pre-cel-miR-84 sequence and engineered Barn H1 and Eco RI sites was synthesized by IDT and cloned into pcDNA3.1(+) vector using restriction sites. Mutations to the pre-miR hairpin are shown in FIG. 6A. All constructs were confirmed by DNA sequencing. To detect pri-miR, ce184U: 5′-GACGGATCCATATTCCTGA-3′ (SEQ ID NO: 117) and ce184D: 5′-GTCGAATTCGTCGTTGTT-3′ (SEQ ID NO: 118). To detect wildtype and M3 cel-pre-miR-84: 5′-TGGCATCTGAGGTAGTATGT-3′ (SEQ ID NO: 119) and 5′-AGAACAGCCGAGTTAGTTGA-3′ (SEQ ID NO: 120). Cel-pre-miR-84M1: 5′-TGGCATCAGACGTAGTATGTAA-3′ (SEQ ID NO: 121) and 5′-AACAGCAGACTTAGTTGAAACAT-3′ (SEQ ID NO:122). Cel-pre-miR-84M2: 5′-GCATCTGAGGCAGACTGTAAT-3′ (SEQ ID NO: 123) and 5′-ACAGCCGAGTCAGACTGAAA-5′ (SEQ ID NO: 124).

Real-time RT-PCR analysis: Total RNA was extracted by TRIzol (Invitrogen) and subjected to reverse transcription using Superscript first-strand cDNA synthesis kit (Invitrogen) according to the manufacturer s instructions. Quantitative analysis of the change in expression levels was calculated by real-time PCR machine (iQ5, Bio-Rad)(Schmittgen et al., 2008). For detection of mature miRNAs, TaqMan MicroRNA assay kit (Applied Biosystems) was used according to manufacturer s instructions. Average of three experiments each performed in triplicate with standard errors of are presented.

In vitro RNA synthesis: 0.5 μg plasmid DNA linearized with XhoI and gel purified was used as a template for in vitro transcription with MAXlscript kit (ambion). The RNA products were treated with IOU DNAse (Roche) and purified using Qiagen RNeasy kit. The amount of transcribed RNA was quantitated by absorbance at 260 nM and the product size and purity was verified by 6% 8M Urea PAGE.

RNA affinity purification: Nuclear extract was prepared from cos7 cells and diluted 1-6 into buffer D (20 mM HEPES-KOH pH 7.6, 5% glycerol, 0.1M KCl, 0.2 mM EDTA, 0.5 mM DTT, 5 U/ml RNAse inhibitor), combined with 50 ug yeast tRNA, and RNA-conjugated beads for 2 hr at 4° C. with rocking. The beads were then washed four times (10 min each) at 4° C. with buffer D. After the final wash, bound proteins were eluted by addition of 50 ul protein sample buffer and heated for 5 min at 95° C. Samples were resolved by 10% SDS PAGE and assayed for the presence of associated proteins assayed by immunoblotting. For competition studies 10 fold molar excess of in vitro transcribed pri-miR or 3-30 fold excess of synthesized DNA oligos was added.

Synthetic RNA/DNA duplex: miR-21 RNA duplex: 5′-UAGCUUAUCAGACUGAUGUUGA-3′ (SEQ ID NO: 73), cel-miR-67 RNA duplex: 5′-UCACAACCUCCUAGAAAGAGUAGA-3′ (SEQ ID NO: 125), SBE1 DNA duplex: 5′-GTATCAGATCTGTGAA-3′ (SEQ ID NO: 126), and SBE2 DNA duplex: 5′-GTATGTCTAGACTGAA-3′ (SEQ ID NO: 127).

This invention is not limited in its application to the details of construction and the arrangement of components set forth in this description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An isolated oligonucleotide comprising a substantially double-stranded portion having the nucleotide sequence CAGRN, wherein R is A or G and N is A, G, C, or U, wherein the isolated oligonucleotide inhibits the binding of a receptor-specific SMAD (rSMAD) protein to an miRNA, and
(i) wherein the isolated oligonucleotide is not a primary miRNA of miR-21, miR-199a, miR-105, miR-509-1(5p), miR-421, or miR-600r,
(ii) wherein the isolated oligonucleotide inhibits the binding of a receptor-specific SMAD (rSMAD) protein to an miRNA, and wherein at least one nucleotide is a modified nucleotide, or
(iii) wherein the isolated oligonucleotide is conjugated in a Nuclear Localization Signal (NLS).

2-5. (canceled)

6. The isolated oligonucleotide of claim 1, wherein the rSMAD protein is selected from SMAD1, SMAD2, SMAD3, SMAD5 and SMAD8.

7. (canceled)

8. The isolated oligonucleotide of claim 1, having a formula selected from:
(SEQ ID NO: 3)
5′- (X¹)₍ᵢ₊ⱼ₎ C A G A C (X²)ₖG U C U G
(X³)₍ᵢ₊ₘ₎ -3′,
(SEQ ID NO: 4)
5′- (X¹)₍ᵢ₊ⱼ₎ G U C U G (X²)ₖC A G A C
(X³)₍ᵢ₊ₘ₎ -3′
and
(SEQ ID NO: 5)
5′- (X¹)₍ᵢ₊ⱼ₎ C A G A C (X²)ₖG U C G
(X³)₍ᵢ₊ₘ₎ -3′,
wherein each of the X¹, X², and X³ is independently any nucleotide, wherein i represents at least one nucleotide, wherein k represents at least one nucleotide, and wherein j and m independently represent zero or more overhang nucleotides, and wherein (X²)ₖ forms a loop structure.

9-16. (canceled)

17. A vector comprising the isolated nucleic acid of claim 8 and an expression sequence.

18. A cell comprising the vector of claim 17.

19. A virus comprising the vector of claim 17, optionally wherein the virus is an adenovirus, a lentivirus, retrovirus, herpesvirus, or adeno-associated virus.

20. The isolated oligonucleotide of claim 1, having a formula selected from:
(SEQ ID NO: 6)
5′- (X¹)₍ᵢ₊ⱼ₎ C A G A C (X³)₍ₖ₊ₘ₎ -3′
(SEQ ID NO: 7)
3′- (X²)₍ᵢ₊ₙ₎ G U C U G (X⁴)₍ₖ₊ₚ₎ -5′,
(SEQ ID NO: 6)
5′- (X¹)₍ᵢ₊ⱼ₎ C A G A C (X³)₍ₖ₊ₘ₎ -3′
(SEQ ID NO: 8)
3′- (X²)₍ᵢ₊ₙ₎ G - C U G (X⁴)₍ₖ₊ₚ₎ -5′,
and
(SEQ ID NO: 6)
5′- (X¹)₍ᵢ₊ⱼ₎ C A G A C (X³)₍ₖ₊ₘ₎ -3′
(SEQ ID NO: 9)
3′- (X²)₍ᵢ₊ₙ₎ - U C U G (X⁴)₍ₖ₊ₚ₎ -5′,
wherein each of X¹, X², X³, and X⁴, is independently any nucleotide, wherein i and k independently represent at least one nucleotide, and wherein j, n, m and p independently represent zero or more overhang nucleotides.

21-38. (canceled)

39. A composition comprising the isolated oligonucleotide of claim 1.

40. (canceled)

41. A method for inhibiting maturation of at least one primary miRNA in a cell, comprising:
contacting the cell with an isolated oligonucleotide of claim 1 wherein the isolated oligonucleotide inhibits the binding of a receptor-specific SMAD (rSMAD) protein to a miRNA.

42-54. (canceled)

55. A method for treating a subject having a TGF-Beta/BMP mediated disorder comprising:
administering to the subject a therapeutically effective amount of an isolated oligonucleotide of claim 1.

56-76. (canceled)

77. A method for treating cancer comprising:
administering to the subject a therapeutically effective amount of an isolated oligonucleotide of claim 1.

78-91. (canceled)

92. A method for treating cancer comprising:
administering to the subject a therapeutically effective amount of a SMAD inhibitor.

93-104. (canceled)

105. A method comprising:
administering to a subject a therapeutically effective amount of an isolated TGF-β/BMP/miR pathway activator in an effective amount to promote wound healing, inhibit scar tissue formation or treat a disorder in the subject, wherein the disorder is a metabolic bone disorder, a fibroproliferative disorder or a smooth muscle cell disorder.

106-131. (canceled)

132. A method of inhibiting microRNA processing, comprising contacting a cell with a SMAD inhibitor in an effective amount to inhibit processing of a TGF microRNA.

133. A method for inhibiting TGF-β signaling comprising contacting a cell with a TGF-β/BMP/miR pathway inhibitor.

134-136. (canceled)

137. An miRNA comprising a heterologous substantially double-stranded portion comprising the nucleotide sequence CAGRN that promotes binding of a receptor-specific SMAD (rSMAD) protein to the miRNA, wherein R is A or G and N is A, G, C, or U.

138-144. (canceled)

145. A synthetic miRNA comprising a seed sequence and a substantially double-stranded portion comprising the nucleotide sequence CAGRN that promotes binding of a receptor-specific SMAD (rSMAD) protein to the synthetic miRNA, wherein R is A or G and N is A, G, C, or U.

146-159. (canceled)

160. An oligonucleotide array for determining levels of miRNAs, the oligonucleotide array consisting essentially of immobilized probes that hybridize with TGF miRNAs, and optionally one or more control probes.

161-166. (canceled)


SEQUENCE LISTING (170 sequences)

SEQ ID NO: 1 - RNA, 7 nt, Artificial Sequence
  Synthetic Oligonucleotide
ncagacn

SEQ ID NO: 2 - RNA, 7 nt, Artificial Sequence
  Synthetic Oligonucleotide
ngucugn

SEQ ID NO: 3 - RNA, 13 nt, Artificial Sequence
  Synthetic Oligonucleotide
ncagacngucugn

SEQ ID NO: 4 - RNA, 13 nt, Artificial Sequence
  Synthetic Oligonucleotide
ngucugncagacn

SEQ ID NO: 5 - RNA, 12 nt, Artificial Sequence
  Synthetic Oligonucleotide
ncagacngucgn

SEQ ID NO: 6 - RNA, 7 nt, Artificial Sequence
  Synthetic Oligonucleotide
ncagacn

SEQ ID NO: 7 - RNA, 7 nt, Artificial Sequence
  Synthetic Oligonucleotide
ngucugn

SEQ ID NO: 8 - RNA, 6 nt, Artificial Sequence
  Synthetic Oligonucleotide
ngcugn

SEQ ID NO: 9 - RNA, 6 nt, Artificial Sequence
  Synthetic Oligonucleotide
nucugn

SEQ ID NO: 10 - protein, 7 aa, Artificial Sequence
  Synthetic Peptide
PKKKRKV

SEQ ID NO: 11 - protein, 16 aa, Artificial Sequence
  Synthetic Peptide
KRPAAIKKAGQAKKKK

SEQ ID NO: 12 - protein, 18 aa, Artificial Sequence
  Synthetic Peptide
RRRHSDENDGGQPHKRRK

SEQ ID NO: 13 - protein, 11 aa, Artificial Sequence
  Synthetic Peptide
RQARRNRRRWE

SEQ ID NO: 14 - protein, 18 aa, Artificial Sequence
  Synthetic Peptide
MPKTRRRPRRSQRKRPPT

SEQ ID NO: 15 - protein, 38 aa, Artificial Sequence
  Synthetic Peptide
NQSSNFGPMKGGNFGGRSSGPYGGGGQYFAKPRNQGGY

SEQ ID NO: 16 - protein, 9 aa, Artificial Sequence
  Synthetic Peptide
PAAKRVKLD

SEQ ID NO: 17 - protein, 43 aa, Artificial Sequence
  Synthetic Peptide
VHSHKKKKIRTSPTFTTPKTLRLRRQPKYPRKSAPRRNKLDHY

SEQ ID NO: 18 - protein, 4 aa, Artificial Sequence
  Synthetic Peptide
KXXX

SEQ ID NO: 19 - protein, 4 aa, Artificial Sequence
  Synthetic Peptide
KRXR

SEQ ID NO: 20 - DNA, 20 bp, Artificial Sequence
  Synthetic Oligonucleotide
ttttgttttgcttgggagga

SEQ ID NO: 21 - DNA, 20 bp, Artificial Sequence
  Synthetic Oligonucleotide
agcagacagtcaggcaggat

SEQ ID NO: 22 - DNA, 20 bp, Artificial Sequence
  Synthetic Oligonucleotide
tgtcgggtagcttatcagac

SEQ ID NO: 23 - DNA, 20 bp, Artificial Sequence
  Synthetic Oligonucleotide
tgtcagacagcccatcgact

SEQ ID NO: 24 - DNA, 20 bp, Artificial Sequence
  Synthetic Oligonucleotide
accacagtccatgccatcac

SEQ ID NO: 25 - DNA, 20 bp, Artificial Sequence
  Synthetic Oligonucleotide
tccaccaccctgttgctgta

SEQ ID NO: 26 - DNA, 22 bp, Artificial Sequence
  Synthetic Oligonucleotide
ccagctatgtgtgaagaagagg

SEQ ID NO: 27 - DNA, 22 bp, Artificial Sequence
  Synthetic Oligonucleotide
gtgatctccttctgcattcggt

SEQ ID NO: 28 - DNA, 20 bp, Artificial Sequence
  Synthetic Oligonucleotide
cccattctgtttcagccagt

SEQ ID NO: 29 - DNA, 20 bp, Artificial Sequence
  Synthetic Oligonucleotide
tgtcgtagagcagcacgttt

SEQ ID NO: 30 - DNA, 20 bp, Artificial Sequence
  Synthetic Oligonucleotide
actcagcttagccaggtgga

SEQ ID NO: 31 - DNA, 20 bp, Artificial Sequence
  Synthetic Oligonucleotide
aagctccttttgtcgttgga

SEQ ID NO: 32 - DNA, 25 bp, Artificial Sequence
  Synthetic Oligonucleotide
tatgatgtggaggaggtggatgtga

SEQ ID NO: 33 - DNA, 25 bp, Artificial Sequence
  Synthetic Oligonucleotide
cctttcatccaaaggcaaaactaca

SEQ ID NO: 34 - DNA, 21 bp, Artificial Sequence
  Synthetic Oligonucleotide
agaggttcaggtcgttccagg

SEQ ID NO: 35 - DNA, 21 bp, Artificial Sequence
  Synthetic Oligonucleotide
ggaatatcctgttggcattgg

SEQ ID NO: 36 - DNA, 24 bp, Artificial Sequence
  Synthetic Oligonucleotide
gagtgtgcagacggaacttcagcc

SEQ ID NO: 37 - DNA, 21 bp, Artificial Sequence
  Synthetic Oligonucleotide
gtctgtgcccagcttggggtc

SEQ ID NO: 38 - DNA, 22 bp, Artificial Sequence
  Synthetic Oligonucleotide
cgcgaagtgcagtccaaaatcg

SEQ ID NO: 39 - DNA, 23 bp, Artificial Sequence
  Synthetic Oligonucleotide
gggctggttcttcttcaatgggc

SEQ ID NO: 40 - RNA, 25 nt, Artificial Sequence
  Synthetic Oligonucleotide
ccugaguauugguguuccauugcuu

SEQ ID NO: 41 - RNA, 25 nt, Artificial Sequence
  Synthetic Oligonucleotide
gcaaaggugugcaguuggaauguaa

SEQ ID NO: 42 - RNA, 25 nt, Artificial Sequence
  Synthetic Oligonucleotide
gcaaccgaguaacugugucaccauu

SEQ ID NO: 43 - RNA, 25 nt, Artificial Sequence
  Synthetic Oligonucleotide
ggucugcaucaaucccuaccacuau

SEQ ID NO: 44 - RNA, 25 nt, Artificial Sequence
  Synthetic Oligonucleotide
gccaccugaugaucagaugggucaa

SEQ ID NO: 45 - RNA, 25 nt, Artificial Sequence
  Synthetic Oligonucleotide
gcuuggguuuguugucaaauguuaa

SEQ ID NO: 46 - RNA, 25 nt, Artificial Sequence
  Synthetic Oligonucleotide
ggaaucuugaugagcugccuaaauu

SEQ ID NO: 47 - RNA, 25 nt, Artificial Sequence
  Synthetic Oligonucleotide
acaacugcccgaagccaguucuaaa

SEQ ID NO: 48 - RNA, 26 nt, Artificial Sequence
  Synthetic Oligonucleotide
ggugcagcaaguagcugcugaauaaa

SEQ ID NO: 49 - DNA, 21 bp, Artificial Sequence
  Synthetic Oligonucleotide
gccaacccagtgttcagacta

SEQ ID NO: 50 - DNA, 21 bp, Artificial Sequence
  Synthetic Oligonucleotide
gcctaaccaatgtgcagacta

SEQ ID NO: 51 - DNA, 20 bp, Artificial Sequence
  Synthetic Oligonucleotide
ccctttccccttactctcca

SEQ ID NO: 52 - DNA, 20 bp, Artificial Sequence
  Synthetic Oligonucleotide
ctatggtgtgagggctgctt

SEQ ID NO: 53 - DNA, 20 bp, Artificial Sequence
  Synthetic Oligonucleotide
gcaagcacatagtggagcaa

SEQ ID NO: 54 - DNA, 20 bp, Artificial Sequence
  Synthetic Oligonucleotide
tcaaacatccaggacaacca

SEQ ID NO: 55 - RNA, 23 nt, Artificial Sequence
  Synthetic Oligonucleotide
gucaacaucagucugauaagcua

SEQ ID NO: 56 - RNA, 23 nt, Artificial Sequence
  Synthetic Oligonucleotide
gaacagguagucugaacacuggg

SEQ ID NO: 57 - RNA, 22 nt, Artificial Sequence
  Synthetic Oligonucleotide
ucacaaguuagggucucaggga

SEQ ID NO: 58 - RNA, 23 nt, Artificial Sequence
  Synthetic Oligonucleotide
gaaacccagcagacaauguagcu

SEQ ID NO: 59 - RNA, 22 nt, Artificial Sequence
  Synthetic Oligonucleotide
uguaaaccaugaugugcugcua

SEQ ID NO: 60 - RNA, 22 nt, Artificial Sequence
  Synthetic Oligonucleotide
cacaaguucggaucuacggguu

SEQ ID NO: 61 - RNA, 21 nt, Artificial Sequence
  Synthetic Oligonucleotide
aaggcaagcugacccugaagu

SEQ ID NO: 62 - RNA, 72 nt, H. Sapiens
ugucggguagcuuaucagacugauguugacuguugaaucucauggcaacaccagucgaugggcugucugaca

SEQ ID NO: 63 - RNA, 71 nt, H. Sapiens
gccaacccaguguucagacuaccuguucaggaggcucucaauguguacaguagucugcacauugguuaggc

SEQ ID NO: 64 - RNA, 110 nt, H. Sapiens
aggaagcuucuggagauccugcuccgucgccccaguguucagacuaccuguucaggacaaugccguuguacaguagucugcacauugguuagacugggcaagggagagca

SEQ ID NO: 65 - RNA, 81 nt, H. Sapiens
ugugcaucguggucaaaugcucagacuccugugguggcugcucaugcaccacggauguuugagcaugugcuacggugucua

SEQ ID NO: 66 - RNA, 94 nt, H. Sapiens
caugcugugugugguacccuacugcagacaguggcaaucauguauaauuaaaaaugauugguacgucuguggguagaguacugcaugacacaug

SEQ ID NO: 67 - RNA, 91 nt, H. Sapiens
caugcugugugugguacccuacugcagacaguggcaaucauguauaauuaaaaaugauugguacgucuguggguagaguacugcaugacac

SEQ ID NO: 68 - RNA, 75 nt, H. Sapiens
gugguacccuacugcagacguggcaaucauguauaauuaaaaaugauugguacgucuguggguagaguacugcau

SEQ ID NO: 69 - RNA, 85 nt, H. Sapiens
gcacauuguaggccucauuaaauguuuguugaaugaaaaaaugaaucaucaacagacauuaauugggcgccugcucugugaucuc

SEQ ID NO: 70 - RNA, 110 nt, H. Sapiens
aucauucagaaaugguauacaggaaaaugaccuaugaauugacagacaauauagcugaguuugucugucauuucuuuaggccaauauucuguaugacugugcuacuucaa

SEQ ID NO: 71 - RNA, 110 nt, H. Sapiens
ggccuggcuggacagaguugucaugugucugccugucuacacuugcugugcagaacauccgcucaccuguacagcaggcacagacaggcagucacaugacaacccagccu

SEQ ID NO: 72 - RNA, 98 nt, H. Sapiens
aagucacgugcuguggcuccagcuucauaggaaggcucuugucugucaggcaguggaguuacuuacagacaagagccuugcucaggccagcccugccc

SEQ ID NO: 73 - RNA, 22 nt, H. Sapiens
uagcuuaucagacugauguuga

SEQ ID NO: 74 - RNA, 22 nt, H. Sapiens
cccaguguucagacuaccuguu

SEQ ID NO: 75 - RNA, 23 nt, H. Sapiens
ucaaaugcucagacuccuguggu

SEQ ID NO: 76 - RNA, 22 nt, H. Sapiens
cuacugcagacaguggcaauca

SEQ ID NO: 77 - RNA, 23 nt, H. Sapiens
aucaacagacauuaauugggcgc

SEQ ID NO: 78 - RNA, 27 nt, H. Sapiens
augaccuaugaauugacagacaauaua

SEQ ID NO: 79 - RNA, 22 nt, H. Sapiens
acagcaggcacagacaggcagu

SEQ ID NO: 80 - RNA, 23 nt, H. Sapiens
acuuacagacaagagccuugcuc

SEQ ID NO: 81 - RNA, 22 nt, H. Sapiens
uagaccuggcccagaccucagc

SEQ ID NO: 82 - RNA, 22 nt, H. Sapiens
uauacaagggcagacucucucu

SEQ ID NO: 83 - RNA, 22 nt, H. Sapiens
aucgaauagucugacuacaacu

SEQ ID NO: 84 - DNA, 20 bp, Artificial Sequence
  Synthetic Oligonucleotide
tgttttgcctaccatcgtga

SEQ ID NO: 85 - DNA, 20 bp, Artificial Sequence
  Synthetic Oligonucleotide
aagtgccaccagacagaagg

SEQ ID NO: 86 - DNA, 19 bp, Artificial Sequence
  Synthetic Oligonucleotide
tgtcagacagcccatcgac

SEQ ID NO: 87 - DNA, 20 bp, Artificial Sequence
  Synthetic Oligonucleotide
aagtgccaccagacagaagg

SEQ ID NO: 88 - DNA, 20 bp, Artificial Sequence
  Synthetic Oligonucleotide
agaaacacagagcacaggaa

SEQ ID NO: 89 - DNA, 21 bp, Artificial Sequence
  Synthetic Oligonucleotide
tgtgcatcgtggtcaaatgct

SEQ ID NO: 90 - DNA, 21 bp, Artificial Sequence
  Synthetic Oligonucleotide
tagacaccgtagcacatgctc

SEQ ID NO: 91 - DNA, 20 bp, Artificial Sequence
  Synthetic Oligonucleotide
gacccccaaagagtcagaca

SEQ ID NO: 92 - DNA, 20 bp, Artificial Sequence
  Synthetic Oligonucleotide
ctctgagcagccaaggaaac

SEQ ID NO: 93 - DNA, 20 bp, Artificial Sequence
  Synthetic Oligonucleotide
actctcatttgattccagca

SEQ ID NO: 94 - DNA, 20 bp, Artificial Sequence
  Synthetic Oligonucleotide
cgtggtgttagtcgatttct

SEQ ID NO: 95 - DNA, 21 bp, Artificial Sequence
  Synthetic Oligonucleotide
atcattcagaaatggtataca

SEQ ID NO: 96 - DNA, 21 bp, Artificial Sequence
  Synthetic Oligonucleotide
ttgaagtagcacagtcataca

SEQ ID NO: 97 - DNA, 20 bp, Artificial Sequence
  Synthetic Oligonucleotide
atcattgtccgtgtctatgg

SEQ ID NO: 98 - DNA, 20 bp, Artificial Sequence
  Synthetic Oligonucleotide
cattctgaagagagcttgga

SEQ ID NO: 99 - DNA, 21 bp, Artificial Sequence
  Synthetic Oligonucleotide
gcacattgtaggcctcattaa

SEQ ID NO: 100 - DNA, 21 bp, Artificial Sequence
  Synthetic Oligonucleotide
gagatcacagagcaggcgccc

SEQ ID NO: 101 - DNA, 20 bp, Artificial Sequence
  Synthetic Oligonucleotide
gcaggaaacataaggaaaga

SEQ ID NO: 102 - DNA, 20 bp, Artificial Sequence
  Synthetic Oligonucleotide
agggtaaaatacctgcactg

SEQ ID NO: 103 - DNA, 21 bp, Artificial Sequence
  Synthetic Oligonucleotide
catgctgtgtgtggtacccta

SEQ ID NO: 104 - DNA, 21 bp, Artificial Sequence
  Synthetic Oligonucleotide
catgtgtcatgcagtactcta

SEQ ID NO: 105 - DNA, 21 bp, Artificial Sequence
  Synthetic Oligonucleotide
ggcctggctggacagagttgt

SEQ ID NO: 106 - DNA, 21 bp, Artificial Sequence
  Synthetic Oligonucleotide
aggctgggttgtcatgtgact

SEQ ID NO: 107 - DNA, 21 bp, Artificial Sequence
  Synthetic Oligonucleotide
gctgctggaaggtgtaggtac

SEQ ID NO: 108 - DNA, 21 bp, Artificial Sequence
  Synthetic Oligonucleotide
agctagaagatgccatcagag

SEQ ID NO: 109 - DNA, 21 bp, Artificial Sequence
  Synthetic Oligonucleotide
tgaacatccaggtctggggca

SEQ ID NO: 110 - DNA, 21 bp, Artificial Sequence
  Synthetic Oligonucleotide
gagaacatgtttccaggtagc

SEQ ID NO: 111 - DNA, 28 bp, Artificial Sequence
  Synthetic Oligonucleotide
ccgggatcctgtttgcctaccatcgtga

SEQ ID NO: 112 - DNA, 30 bp, Artificial Sequence
  Synthetic Oligonucleotide
cggaattctgagaacattggatatggatgg

SEQ ID NO: 113 - DNA, 29 bp, Artificial Sequence
  Synthetic Oligonucleotide
tgtcgggtagcttataaaactgatgttga

SEQ ID NO: 114 - DNA, 21 bp, Artificial Sequence
  Synthetic Oligonucleotide
ggcaacacctaacgatgggct

SEQ ID NO: 115 - DNA, 21 bp, Artificial Sequence
  Synthetic Oligonucleotide
cgggttaattatcagactgat

SEQ ID NO: 116 - DNA, 21 bp, Artificial Sequence
  Synthetic Oligonucleotide
gactgttgaccatcatggcaa

SEQ ID NO: 117 - DNA, 19 bp, Artificial Sequence
  Synthetic Oligonucleotide
gacggatccatattcctga

SEQ ID NO: 118 - DNA, 18 bp, Artificial Sequence
  Synthetic Oligonucleotide
gtcgaattcgtcgttgtt

SEQ ID NO: 119 - DNA, 20 bp, Artificial Sequence
  Synthetic Oligonucleotide
tggcatctgaggtagtatgt

SEQ ID NO: 120 - DNA, 20 bp, Artificial Sequence
  Synthetic Oligonucleotide
agaacagccgagttagttga

SEQ ID NO: 121 - DNA, 22 bp, Artificial Sequence
  Synthetic Oligonucleotide
tggcatcagacgtagtatgtaa

SEQ ID NO: 122 - DNA, 23 bp, Artificial Sequence
  Synthetic Oligonucleotide
aacagcagacttagttgaaacat

SEQ ID NO: 123 - DNA, 21 bp, Artificial Sequence
  Synthetic Oligonucleotide
gcatctgaggcagactgtaat

SEQ ID NO: 124 - DNA, 20 bp, Artificial Sequence
  Synthetic Oligonucleotide
acagccgagtcagactgaaa

SEQ ID NO: 125 - RNA, 24 nt, C. elegans
ucacaaccuccuagaaagaguaga

SEQ ID NO: 126 - DNA, 16 bp, Artificial Sequence
  Synthetic Oligonucleotide
gtatcagatctgtgaa

SEQ ID NO: 127 - DNA, 16 bp, Artificial Sequence
  Synthetic Oligonucleotide
gtatgtctagactgaa

SEQ ID NO: 128 - RNA, 68 nt, H. Sapiens
ucggguagcuuaucagacugauguugacuguugaccaucauggcaacaccagucgaugggcugucuga

SEQ ID NO: 129 - RNA, 68 nt, Artificial Sequence
  Synthetic Oligonucleotide
ucggguagcuuauaaaacugauguugacuguugaaucucauggcaacaccagucgaugggcugucuga

SEQ ID NO: 130 - RNA, 68 nt, Artificial Sequence
  Synthetic Oligonucleotide
ucggguagcuuaucagacugauguugacuguugaaucucauggcaacaccuaacgaugggcugucuga

SEQ ID NO: 131 - RNA, 68 nt, Artificial Sequence
  Synthetic Oligonucleotide
ucggguagcuuauaaaacugauguugacuguugaaucugauggcaacaccaguuuaugggcugucuga

SEQ ID NO: 132 - RNA, 68 nt, Artificial Sequence
  Synthetic Oligonucleotide
ucggguuaauuaucagacugauguugacuguugaaucugauggcaacaccagucgaugggcugucuga

SEQ ID NO: 133 - RNA, 68 nt, Artificial Sequence; identical to SEQ ID NO: 128
  Synthetic Oligonucleotide

SEQ ID NO: 134 - RNA, 72 nt, H. Sapiens; identical to SEQ ID NO: 62

SEQ ID NO: 135 - RNA, 72 nt, Artificial Sequence
  Synthetic Oligonucleotide
ugucggguagcuuauaaaacugauguugacuguugaaucucauggcaacaccagucgaugggcugucugaca

SEQ ID NO: 136 - RNA, 72 nt, Artificial Sequence
  Synthetic Oligonucleotide
ugucggguagcuuaucagacugauguugacuguugaaucucauggcaacaccuaacgaugggcugucugaca

SEQ ID NO: 137 - RNA, 72 nt, Artificial Sequence
  Synthetic Oligonucleotide
ugucggguagcuuauaaaacugauguugacuguugaaucucauggcaacaccaguuuaugggcugucugaca

SEQ ID NO: 138 - RNA, 72 nt, Artificial Sequence
  Synthetic Oligonucleotide
ugucggguuaauuaucagacugauguugacuguugaaucucauggcaacaccagucgaugggcugucugaca

SEQ ID NO: 139 - RNA, 72 nt, Artificial Sequence
  Synthetic Oligonucleotide
ugucggguagcuuaucagacugauguugacuguugaccaucauggcaacaccagucgaugggcugucugaca

SEQ ID NO: 140 - RNA, 22 nt, H. Sapiens
ucaacaucagucugauaagcua

SEQ ID NO: 141 - RNA, 24 nt, C. elegans
ucuacucuuucuaggagguuguga

SEQ ID NO: 142 - DNA, 16 bp, Artificial Sequence
  Synthetic Oligonucleotide
ttcacagatctgatac

SEQ ID NO: 143 - DNA, 16 bp, Artificial Sequence
  Synthetic Oligonucleotide
ttcagtctagacatac

SEQ ID NO: 144 - RNA, 75 nt, C. elegans
guggcaucugagguaguauguaauauuguagacugucuauaauguccacaauguuucaacuaacucggcuguucu

SEQ ID NO: 145 - RNA, 75 nt, Artificial Sequence
  Synthetic Oligonucleotide
guggcaucagacguaguauguaauauuguagacugucuauaauguccacaauguuucaacuaagucugcuguucu

SEQ ID NO: 146 - RNA, 76 nt, Artificial Sequence
  Synthetic Oligonucleotide
guggcaucugaggcagacuguaauauuguagacugucuauaauguccacaauguuucagucugacucggcuguucu

SEQ ID NO: 147 - RNA, 75 nt, Artificial Sequence
  Synthetic Oligonucleotide
guggcaucugagguaguauguaauauugcagacugucuauaaugucugcaauguuucaacuaacucggcuguucu

SEQ ID NO: 148 - RNA, 70 nt, H. Sapiens
gucggguagcuuaucagacagauguugacuguugaaucucauggcaacaccagucgaugggcugucugac

SEQ ID NO: 149 - RNA, 71 nt, H. Sapiens; identical to SEQ ID NO: 63

SEQ ID NO: 150 - RNA, 104 nt, H. Sapiens
gcuucuggagauccugcuccgucgccccaguguucauacuaccuguucaggacaaugccguuguacaguagucugcacauugguuagacugggcaagggagagc

SEQ ID NO: 151 - RNA, 75 nt, H. Sapiens
gcaucguggucaaaugcucagacuccugugguggcugcucaugcaccacggauguuugagcaugugcuacggugu

SEQ ID NO: 152 - RNA, 100 nt, H. Sapiens
gaaaugguauacaggaaaaugaccuaugaauugacagacaauauagcugaguuugucugucauuucuuuaggccaauauucuguaugacugugcuacuuc

SEQ ID NO: 153 - RNA, 79 nt, H. Sapiens
cacauuguaggccucauuaaauguuuguugaaugaaaaaaugaaucaucaacagacauuaauugggcgccugcucugug

SEQ ID NO: 154 - RNA, 94 nt, H. Sapiens
caugcugugugugguacccaacugcagacagaggcaaucauguauaauuaaaaaugauugguacgucugaggguagaguacugcaugacacaug

SEQ ID NO: 155 - RNA, 85 nt, H. Sapiens
ugugugugguacccaacugcagacaguggcaaucauguauaauuaaaaaugauuggaacgucuguggguagaguacugcaugaca

SEQ ID NO: 156 - RNA, 73 nt, H. Sapiens
gugguacccaacugcagacguggcaaucauguauaauuaaaaaugauugguacgucuguggguagaguacugc

SEQ ID NO: 157 - RNA, 87 nt, H. Sapiens
gcuguggcuccagcuucauaggaaggcucuugucugucaggcaguggaguuacuuacagacaagagccuugcucaggccagcccugc

SEQ ID NO: 158 - RNA, 75 nt, H. Sapiens
guggggagccugguuagaccuggcccagaccucagcuacacaagcugauggacugagucaggggccacacucucc

SEQ ID NO: 159 - RNA, 83 nt, H. Sapiens
ugcuacuugaagagagguaauccuucacgcauuugcuuuacuugcaaugauuauacaagggcagacucucucuggggagcaaa

SEQ ID NO: 160 - RNA, 109 nt, M. Musculus
gccacagcaggugcuuauauugcuucccagacggugaagaaaguaauagagaucaacccguaccuucugggcaccauggcugggggugcagcggauugcagcuucuggg

SEQ ID NO: 161 - RNA, 109 nt, M. Musculus
uuguccgcuaucacuguaccgucauuuuucagucucagacagagauaccuucucugaauucauagaagcugcucuccguuccgaagggauucagaagugauaaauccag

SEQ ID NO: 162 - RNA, 77 nt, M. Musculus
ugcagugcuguguucagacugguguccaucaugugaaauauuugugaaagacaucaugcugaauagaguaaggccca

SEQ ID NO: 163 - RNA, 86 nt, M. Musculus
uuguagggcuccaccacagugucagacacuuugggugagggcaccacacugaagguguucaugaugcggucgggauacuccucacg

SEQ ID NO: 164 - RNA, 80 nt, M. Musculus
uguguuuucgugugugugcauguggauguauguauaugauuuugcauauacagacacaugcacacacaugcggcacacac

SEQ ID NO: 165 - RNA, 71 nt, H. Sapiens
uguucuaaggugcaucuagugcagauagugaaguagauuagcaucuacugcccuaagugcuccuucuggca

SEQ ID NO: 166 - RNA, 82 nt, H. Sapiens
ccugccggggcuaaagugcugacagugcagauagugguccucuccgugcuaccgcacuguggguacuugcugcuccagcagg

SEQ ID NO: 167 - RNA, 80 nt, H. Sapiens
gguaccugagaagagguugucugugaugaguucgcuuuuauuaaugacgaauauaacacagauggccuguuuucaguacc

SEQ ID NO: 168 - RNA, 97 nt, H. Sapiens
cagaucucagacaucucggggaucaucaugucacgagauaccagugugcacuugugacagauugauaacugaaaggucugggagccacucaucuuca

SEQ ID NO: 169 - RNA, 96 nt, H. Sapiens
uugccuaaagucacacagguuauagaucuggauuggaacccagggagccagacugccuggguucaaauccagaucuauaacuugugugacuuuggg

SEQ ID NO: 170 - RNA, 77 nt, H. Sapiens
ugcagugcucuauucagauuagugccagucaugugaaauacauaugacuggcaccauucuggauaauguaaugcuca